(19) United States
(12) Patent Application Publication
(10) Pub. No.: US 2005/0019266 A1
(43) Pub. Date: Jan. 27, 2005

(54) Novel targeted compositions for diagnostic and therapeutic use

(76) Inventors: Evan C. Unger, Tucson, AZ (US); Thomas P. McCreery, Alexandria, VA (US)

Correspondence Address: David A Cherry;Woodcock Washburn, 46th Floor, One Liberty Place, Philadelphia, PA, 19103, US

(21) Appl. No.: 10/502,271

(22) PCT Filed: Jan. 23, 2003
(86) PCT No.: PCT/US03/02024

Related U.S. Application Data
(63) Continuation-in-part of application No. 10/055,772, filed on Jan. 23, 2002, now abandoned.
Application No. 10/055,772 is a continuation-in-part of application No. 09/699,679, filed on Oct. 30, 2000.
Application No. 10/055,772 is a continuation-in-part of application No. 09/496,761, filed on Feb. 3, 2000, now Pat. No. 6,444,660.
Application No. 09/496,761 is a division of application No. 08/851,780, filed on May 6, 1997, now Pat. No. 6,090,800.

(51) Int. Cl. A61K 49/00

(52) U.S. Cl. 424/9.321

(57) ABSTRACT

Novel targeted compositions which may be used for diagnostic and therapeutic use. The compositions can be used in conjunction with diagnostic imaging, such as ultrasound, as well as therapeutic applications, such as therapeutic ultrasound.

### CROSS-REFERENCE TO RELATED APPLICATIONS

This application is a continuation-in-part of U.S. application Ser. No. 10/055,772, filed Jan. 23, 2002, which is a continuation-in-part of U.S. application Ser. No. 09/699,679, filed Oct. 30, 2000. This application is also a continuation-in-part of U.S. application Ser. No. 09/496,761, filed Feb. 3, 2000, which is a divisional of U.S. application Ser. No. 08/851,780, filed May 6, 1997, now U.S. Pat. No. 6,090,800. The disclosures of each of the foregoing applications are hereby incorporated herein by reference, in their entireties.

### FIELD OF THE INVENTION

The present invention relates to novel targeted compositions and the use thereof. More particularly, the present invention relates to novel compounds and targeted compositions comprising those compounds, and methods for using those compositions for diagnostic imaging and/or for the administration of bioactive agents.

### BACKGROUND OF THE INVENTION

A variety of imaging techniques have been used to diagnose diseases. Included among these imaging techniques is X-ray imaging. In X-rays, the images produced reflect the different densities of structures and tissue in the body of the patient. To improve the diagnostic usefulness of this imaging technique, contrast agents may be employed to increase the density of tissues of interest relative to surrounding tissues. Examples of such contrast agents include, for example, barium and iodinated compounds, which may be used for X-ray studies of the gastrointestinal region, including the esophagus, stomach, intestines and rectum. Contrast agents may also be used for computed tomography (CT) and computer assisted tomography (CAT) studies to improve visualization of tissue of interest, for example, the gastrointestinal tract.

Magnetic resonance imaging (MRI) is another imaging technique which, unlike X-rays, does not involve ionizing radiation. MRI may be used for producing cross-sectional images of the body in a variety of scanning planes such as, for example, axial, coronal, sagittal or orthogonal. MRI employs a magnetic field, radio frequency energy and magnetic field gradients to make images of the body. The contrast or signal intensity differences between tissues mainly reflect the T1 (longitudinal) and T2 (transverse) relaxation values and the proton density, which generally corresponds to the free water content, of the tissues. To change the signal intensity in a region of a patient by the use of a contrast medium, several possible approaches are available. For example, a contrast medium may be designed to change either the T1, the T2 or the proton density.

Generally speaking, MRI requires the use of contrast agents. If MRI is performed without employing a contrast agent, differentiation of the tissue of interest from the surrounding tissues in the resulting image may be difficult. In the past, attention has focused primarily on paramagnetic contrast agents for MRI. Paramagnetic contrast agents involve materials which contain unpaired electrons. The unpaired electrons act as small magnets within the main magnetic field to increase the rate of longitudinal (T1) and transverse (T2) relaxation. Paramagnetic contrast agents typically comprise metal ions, for example, transition metal ions, which provide a source of unpaired electrons. However, these metal ions are also generally highly toxic. In an effort to decrease toxicity, the metal ions are typically chelated with ligands.

Metal oxides, most notably iron oxides, have also been employed as MRI contrast agents. While small particles of iron oxide, for example, particles having a diameter of less than about 20 nm, may have desirable paramagnetic relaxation properties, their predominant effect is through bulk susceptibility. Nitroxides are another class of MRI contrast agent which are also paramagnetic. These have relatively low relaxivity and are generally less effective than paramagnetic ions.

The existing MRI contrast agents suffer from a number of limitations. For example, increased image noise may be associated with certain contrast agents, including contrast agents involving chelated metals. This noise generally arises out of intrinsic peristaltic motions and motions from respiration or cardiovascular action. In addition, the signal intensity for contrast agents generally depends upon the concentration of the agent as well as the pulse sequence employed. Absorption of contrast agents can complicate interpretation of the images, particularly in the distal portion of the small intestine, unless sufficiently high concentrations of the paramagnetic species are used. See, e.g., Kornmesser et al., Magnetic Resonance Imaging, 6:124 (1988).

Other contrast agents may be less sensitive to variations in pulse sequence and may provide more consistent contrast. However, high concentrations of particulates, such as ferrites, can cause magnetic susceptibility artifacts which are particularly evident, for example, in the colon where the absorption of intestinal fluid occurs and the superparamagnetic material may be concentrated.

Toxicity is another problem which is generally associated with currently available contrast agents, including contrast agents for MRI. For example, ferrites often cause symptoms of nausea after oral administration, as well as flatulence and a transient rise in serum iron. The gadolinium ion, which is complexed in Gd-DTPA, is highly toxic in free form. The various environments of the gastrointestinal tract, including increased acidity (lower pH) in the stomach and increased alkalinity (higher pH) in the intestines, may increase the likelihood of decoupling and separation of the free ion from the complex.

Ultrasound is another valuable diagnostic imaging technique for studying various areas of the body, including, for example, the vasculature, such as tissue microvasculature. Ultrasound provides certain advantages over other diagnostic techniques. For example, diagnostic techniques involving nuclear medicine and X-rays generally involves exposure of the patient to ionizing electron radiation. Such radiation can cause damage to subcellular material, including deoxyribonucleic acid (DNA), ribonucleic acid (RNA) and proteins. Ultrasound does not involve such potentially damaging radiation. In addition, ultrasound is relatively inexpensive relative to other diagnostic techniques, including CT and MRI, which require elaborate and expensive equipment.

Ultrasound involves the exposure of a patient to sound waves. Generally, the sound waves dissipate due to absorption by body tissue, penetrate through the tissue or reflect off of the tissue. The reflection of sound waves off of tissue, generally referred to as backscatter or reflectivity, forms the basis for developing an ultrasound image. In this connection, sound waves reflect differentially from different body tissues. This differential reflection is due to various factors, including the constituents and the density of the particular tissue being observed. Ultrasound involves the detection of the differentially reflected waves, generally with a transducer that can detect sound waves having a frequency of one megahertz (MHZ) to ten MHZ. The detected waves can be integrated into an image which is quantitated and the quantitated waves converted into an image of the tissue being studied.

As with the diagnostic techniques discussed above, ultrasound also generally involves the use of contrast agents. Exemplary contrast agents include, for example, suspensions of solid particles, emulsified liquid droplets, and gas-filled bubbles. See, e.g., Hilmann et al., U.S. Pat. No. 4,466,442, and published International Patent Applications WO 92/17212 and WO 92/21382. Widder et al., published application EP-A-0 324 938, discloses stabilized microbubble-type ultrasonic imaging agents produced from heat-denaturable biocompatible protein, for example, albumin, hemoglobin, and collagen.

The quality of images produced from ultrasound has improved significantly. Nevertheless, further improvement is needed, particularly with respect to images involving vasculature in tissues that are perfused with a vascular blood supply. Accordingly, there is a need for improved ultrasound techniques, including improved contrast agents which are capable of providing medically useful images of the vasculature and vascular-related organs.

The reflection of sound from a liquid-gas interface is extremely efficient. Accordingly, bubbles, including gas-filled bubbles, are useful as contrast agents. The term “bubbles”, as used herein, refers to vesicles which are generally characterized by the presence of one or more membranes or walls surrounding an internal void that is filled with a gas or precursor thereto. Exemplary bubbles include, for example, liposomes, micelles and the like. As discussed more fully hereinafter, the effectiveness of bubbles as contrast agents depends upon various factors, including, for example, the size and/or elasticity of the bubble.

With respect to the effect of bubble size, the following discussion is provided. As known to the skilled artisan, the signal which is reflected off of a bubble is a function of the radius (r⁶) of the bubble (Rayleigh Scatterer). Thus, in the frequency range of diagnostic ultrasound, a bubble having a diameter of 4 micrometer (μm) possesses about 64 times the scattering ability of a bubble having a diameter of 2 μm. Thus, generally speaking, the larger the bubble, the greater the reflected signal.

However, bubble size is limited by the diameter of capillaries through which the bubbles must pass. Generally, contrast agents which comprise bubbles having a diameter of greater than 10 μm can be dangerous since microvessels may be occluded. Accordingly, it is desired that greater than about 99% of the bubbles in a contrast agent have a diameter of less than 10 μm. Mean bubble diameter is important also, and should be greater than 1 μm, with greater than 2 μm being preferred. The volume weighted mean diameter of the bubbles should be about 7 to 10 micrometer.

The elasticity of bubbles is also important. This is because highly elastic bubbles can deform, as necessary, to “squeeze” through capillaries and/or to permit the flow of blood around the bubbles. This decreases the likelihood of occlusion. The effectiveness of a contrast agent which comprises bubbles is also dependent on the bubble concentration. Generally, the higher the bubble concentration, the greater the reflectivity of the contrast agent.

Another important characteristic which is related to the effectiveness of bubbles as contrast agents is bubble stability. As used herein, particularly with reference to gas-filled bubbles, “bubble stability” refers to the ability of bubbles to retain gas entrapped therein after exposure to a pressure greater than atmospheric pressure. To be effective as contrast agents, bubbles generally need to retain greater than 50% of entrapped gas after exposure to pressure of 300 millimeters (mm) of mercury (Hg) for about one minute. Particularly effective bubbles retain 75% of the entrapped gas after being exposed for one minute to a pressure of 300 mm Hg, with an entrapped gas content of 90% providing especially effective contrast agents. It is also highly desirable that, after release of the pressure, the bubbles return to their original size. This is referred to generally as “bubble resilience.”

Bubbles which lack desirable stability provide poor contrast agents. If, for example, bubbles release the gas entrapped therein in vivo, reflectivity is diminished. Similarly, the size of bubbles which possess poor resilience will be decreased in vivo, also resulting in diminished reflectivity.

The stability of bubbles disclosed in the prior art is generally inadequate for use as contrast agents. For example, the prior art discloses bubbles, including gas-filled liposomes, which comprise lipid-containing walls or membranes. See, e.g., Ryan et al., U.S. Pat. Nos. 4,900,540 and 4,544,545; Tickner et al., U.S. Pat. No. 4,276,885; Klaveness et al., WO 93/13809 and Schneider et al., EPO 0 554 213 and WO 91/15244. Lanza et al., WO 93/20802 discloses acoustically reflective oligolamellar liposomes, which are multilamellar liposomes with increased aqueous space between bilayers or have liposomes nested within bilayers in a nonconcentric fashion, and thus contain internally separated bilayers. Their use as ultrasonic contrast agents to enhance ultrasonic imaging, and in monitoring a drug delivered in a liposome administered to a patient, is also described. D'Arrigo, U.S. Pat. Nos. 4,684,479 and 5,215,680 disclose gas-in-liquid emulsions and lipid-coated microbubbles, respectively.

Many of the bubbles disclosed in the prior art have undesirably poor stability. Thus, the prior art bubbles are more likely to rupture in vivo resulting, for example, in the untimely release of any therapeutic and/or diagnostic agent contained therein. Various studies have been conducted in an attempt to improve bubble stability. Such studies have included, for example, the preparation of bubbles in which the membranes or walls thereof comprise proteins, such as albumin, or materials which are apparently strengthened via crosslinking. See, e.g., Klaveness et al., WO 92/17212, in which there are disclosed bubbles which comprise proteins crosslinked with biodegradable crosslinking agents. A presentation was made by Moseley et al., at a 1991 Napa, California meeting of the Society for Magnetic Resonance in Medicine, which is summarized in an abstract entitled “Microbubbles: A Novel MR Susceptibility Contrast Agent.” The microbubbles described by Moseley et al. comprise air coated with a shell of human albumin. Alternatively, bubble membranes can comprise compounds which are not proteins but which are crosslinked with biocompatible compounds. See, e.g., Klaveness et al., WO 92/17436, WO 93/17718 and WO 92/21382.

Prior art techniques for stabilizing bubbles, including the use of proteins in the outer membrane or crosslinking of the membrane components, suffer from various drawbacks. For example, the crosslinking described above generally involves the use of new materials, including crosslinked proteins or other compounds, for which the metabolic fate is unknown. In addition, crosslinking requires additional chemical process steps, including isolation and purification of the crosslinked compounds. Moreover, the use in bubble membranes of proteins, such as albumin, and crosslinking of the bubble membrane components, can impart rigidity to the walls of the bubbles. This results in bubbles having reduced elasticity and, therefore, a decreased ability to deform and pass through capillaries. Thus, there is a greater likelihood of occlusion of vessels with prior art contrast agents that involve proteins and/or crosslinking.

Vascular plaque is a primary indicator associated with vascular blockages which may cause heart attacks, stroke and other vascular diseases. Plaque may be the single most important cause of disease in the industrialized world. Investigations on the molecular characterization of plaque continue to be the focal point for the development of therapeutic strategies for treating vascular disease.

So called “scavenger receptors” expressed on macrophages take up lipoproteins, particulary LDL. Macrophages containing phagocytosed lipoproteins resemble and are closely related to the foam cells associated with atherosclerotic lesions. Of the known scavenger receptors in the literature, at least one, SR-PSOX, has affinity for ligands composed of phosphoserine or a closely related analog. Receptor-mediated endocytosis of oxidized low density lipoprotein (Ox-LDL) by macrophages and the subsequent foam cell transformation in the arterial endothelium characterize the early stages of atherosclerosis. A macrophage cell-surface receptor for Ox-LDL, designated (SR-PSOX), which functions as a receptor for phosphatidylserine has been identified (Minami, et al., Arterioscler. Thromb. Vasc. Biol. (2001) 21:1796-1800.) This receptor was shown to be prominent in atherosclerotic lesions but not in normal endothelium.

The incorporation of phosphoserine and phosphatidylserine into liposomes and microbubbles has been reported in the literature. For example, according to Lindner, et al., (Circulation (2000) 102:2745-2750), the incorporation of phosphatidylserine into the shells of microbubbles may enhance attachment to leukocytes within venules by amplifying complement activation and thereby allow ultrasound imaging of inflammation.

Plaques also contain lipids and rapidly accumulate macrophages as a cell-mediated endocytotic response by the body to the lesion. Therefore, a targeted therapeutic agent that may be biocompatible with plaque lipids and designed to be endocytosed by the macrophages may advantageously provide for the delivery of therapeutic agents to the plaque, for example, antithrombotics and clot dissolution agents, as well as provide a target for mechanical disruption of the plaque.

Various other receptors have been associated with the surfaces of atherosclerotic plaques and are amenable to targeting either for diagnostic or therapeutic purposes. Platelet-activating growth factor receptor (PAF-R) and platelet derived growth factor (PDGF) receptor expression is modulated by cytokines and lipoprotein levels. Symptoms of stenosis have been correlated with the expression of the latter on plaques. Elastin-laminin receptor, angiotensin II and alphaᵥ-beta₃ or alphaᵥ-beta₅ have also been correlated with atherosclerosis.

Monocyte and macrophage homing responses are additional aspects of plaque formation that have been exploited. In this regard, liposomes coated with fibronectin have been shown to be phagocytosed more readily by plaque-associated macrophages than non-coated liposomes. Endothelin-A receptor antagonists interfere with monocyte and macrophage homing, implying a role for endothelin as a plaque targeting ligand.

Bioactive agents effective in interfering with the progression of events leading to the maturation of vascular plaques, particularly the statins, HMGCoA reductase inhibitors, have been shown to act both to decrease the cholesterol content of LDL and to modify characteristics of the arterial wall endothelium. Delivery of statins to the site of vascular plaques would, therefore, be highly therapeutically valuable therapeutically.

Accordingly, new and/or better stabilized contrast agents and methods involving same, and new and/or improved methods for delivering bioactive agents to specific regions of interest, are needed. The present invention is directed to these, as well as other, important ends.

### SUMMARY OF THE INVENTION

The present invention is directed, in part, to improved contrast agents and methods for enhancing the delivery of bioactive agents. Specifically, in one embodiment, there is provided a method for providing an image of an internal region of a patient having a vascular plaque, wherein the method comprises (i) administering to the patient a contrast agent comprising, in an aqueous carrier, targeted vesicles formulated from a lipid or protein, a gas or gaseous precursor, a targeting ligand, and optionally, an oil, wherein said targeting ligand targets cells or receptors associated with vascular plaque and comprises an acid moiety; and (ii) scanning the patient using ultrasound to obtain a visible image of the region.

Another embodiment of the invention relates to a method for diagnosing the presence of a vascular plaque in a patient, wherein the method comprises (i) administering to the patient a contrast agent comprising, in an aqueous carrier, targeted vesicles formulated from a lipid or protein, a gas or gaseous precursor, a targeting ligand, and optionally, an oil, wherein said targeting ligand targets cells or receptors associated with vascular plaque and comprises an acid moiety; and (ii) scanning the patient using ultrasound to obtain a visible image of any plaque in the patient.

Yet another embodiment of the invention relates to a method for the therapeutic delivery in vivo of a bioactive agent to a region in a patient having a vascular plaque, wherein the method comprises administering to a patient a therapeutically effective amount of a formulation comprising, in combination with a bioactive agent, a composition which comprises vesicles formulated from a lipid or protein, a gas or gaseous precursor, a targeting ligand, and optionally, an oil, wherein said targeting ligand targets cells or receptors associated with vascular plaque and comprises an acid moiety.

Still another embodiment of the invention relates to a method of dissolving plaque in a blood vessel comprising (i) administering to a patient, by intravenous injection, a targeted vesicle composition comprising vesicles formulated from a lipid or protein, a gas or gaseous precursor, a targeting ligand, and optionally, an oil, wherein said targeting ligand targets cells or receptors associated with vascular plaque and comprises an acid moiety; (ii) scanning said patient with diagnostic imaging to visualize said plaque; and (iii) applying ultrasonic energy to said plaque.

Another embodiment of the invention relates to a method for providing an image of an internal region of a patient having a vascular plaque, wherein the method comprises (i) administering to the patient a contrast agent comprising, in an aqueous carrier, targeted vesicles formulated from a lipid or polymer, a gas or gaseous precursor, a targeting ligand, and optionally, an oil, wherein said targeting ligand targets cells or receptors associated with vascular plaque and comprises a phosphorylated serine moiety; and (ii) scanning the patient using ultrasound to obtain a visible image of the region.

Yet another embodiment of the invention relates to a method for diagnosing the presence of a vascular plaque in a patient, wherein the method comprises (i) administering to the patient a contrast agent comprising, in an aqueous carrier, targeted vesicles formulated from a lipid or polymer, a gas or gaseous precursor, a targeting ligand, and optionally, an oil, wherein said targeting ligand targets cells or receptors associated with vascular plaque and comprises a phosphorylated serine moiety; and (ii) scanning the patient using ultrasound to obtain a visible image of any plaque in the patient.

Still another embodiment of the invention relates to a method for the therapeutic delivery in vivo of a bioactive agent to a region in a patient having a vascular plaque, wherein the method comprises administering to a patient a therapeutically effective amount of a formulation comprising, in combination with a bioactive agent, a composition which comprises vesicles formulated from a lipid or polymer, a gas or gaseous precursor, a targeting ligand, and optionally, an oil, wherein said targeting ligand targets cells or receptors associated with vascular plaque and comprises a phosphorylated serine moiety.

Another embodiment of the invention relates to a composition for use in targeting an internal region of a patient having vascular plaque, wherein the composition comprises vesicles formulated from a lipid or protein, a gas or gaseous precursor, a targeting ligand, and optionally, an oil, wherein said targeting ligand targets cells or receptors associated with vascular plaque and comprises an acid moiety.

Yet another embodiment of the invention relates to a composition for use in targeting an internal region of a patient having vascular plaque, wherein the composition comprises vesicles formulated from a lipid or polymer, a gas or gaseous precursor, a targeting ligand, and optionally, an oil, wherein said targeting ligand targets cells or receptors associated with vascular plaque and comprises a phosphorylated serine moiety.

Still another embodiment of the invention relates to a formulation for therapeutic or diagnostic use in a patient having a vascular plaque, wherein the formulation comprises, in combination with a bioactive agent, a composition comprising vesicles formulated from a lipid or protein, a gas or gaseous precursor, a targeting ligand, and optionally, an oil, wherein said targeting ligand targets cells or receptors associated with vascular plaque and comprises an acid moiety.

Another embodiment of the invention relates to a formulation for therapeutic or diagnostic use in a patient having a vascular plaque, wherein the formulation comprises, in combination with a bioactive agent, a composition comprising vesicles formulated from a lipid or polymer, a gas or gaseous precursor, a targeting ligand, and optionally, an oil, wherein said targeting ligand targets cells or receptors associated with vascular plaque and comprises a phosphorylated serine moiety.

Yet another embodiment of the invention relates to a process for the preparation of a composition for use in targeting a region in a patient having a vascular plaque, wherein the process comprises combining together a lipid or protein, a gas or gaseous precursor, a targeting ligand, and optionally, an oil, wherein said targeting ligand targets cells or receptors associated with vascular plaque and comprises an acid moiety.

Still another-embodiment a process for the preparation of a composition for use in targeting a region in a patient having a vascular plaque, wherein the process comprises combining together a lipid or polymer, a gas or gaseous precursor, a targeting ligand, and optionally, an oil, wherein said targeting ligand targets cells or receptors associated with vascular plaque and comprises a phosphorylated serine moiety.

Another embodiment of the invention relates to a process for the preparation of a formulation for therapeutic or diagnostic use in a patient having a vascular plaque, wherein the process comprises combining together a bioactive agent, a lipid or protein, a gas or gaseous precursor, a targeting ligand, and optionally, an oil, wherein said targeting ligand targets cells or receptors associated with vascular plaque and comprises an acid moiety.

Yet another embodiment of the invention relates to a process for the preparation of a formulation for therapeutic or diagnostic use in a patient having a vascular plaque, wherein the process comprises combining together a bioactive agent, a lipid or polymer, a gas or gaseous precursor, a targeting ligand, and optionally, an oil, wherein said targeting ligand targets cells or receptors associated with vascular plaque and comprises a phosphorylated serine moiety.

These and other aspects of the invention will become more apparent from the following detailed description

### DETAILED DESCRIPTION OF THE INVENTION

As employed above and throughout the disclosure, the following terms, unless otherwise indicated, shall be understood to have the following meanings.

“Lipid” refers to a synthetic or naturally-occurring compound which is generally amphipathic and biocompatible. The lipids typically comprise a hydrophilic component and a hydrophobic component. Exemplary lipids include, for example, fatty acids, neutral fats, phosphatides, glycolipids, surface-active agents (surfactants), aliphatic alcohols, waxes, terpenes and steroids. In certain preferred embodiments, the lipids which may be incorporated in the compositions described herein contain no sulfhydryl groups or disulfide linkages.

“Lipid composition” refers to a composition which comprises a lipid compound, typically in an aqueous medium. Exemplary lipid compositions include suspensions, emulsions and vesicle compositions.

“Lipid formulation” refers to a lipid composition which also comprises a bioactive agent.

“Vesicle” refers to a spherical entity which is generally characterized by the presence of one or more walls or membranes which form one or more internal voids. Vesicles may be formulated, for example, from lipids, including the various lipids described herein, proteinaceous materials, or polymeric materials, including natural, synthetic and semi-synthetic polymers. Preferred vesicles are those which comprise walls or membranes formulated from lipids. In these preferred vesicles, the lipids may be in the form of a monolayer or bilayer, and the mono- or bilayer lipids may be used to form one or more mono- or bilayers. In the case of more than one mono- or bilayer, the mono- or bilayers may be concentric. Lipids may be used to form a unilamellar vesicle (comprised of one monolayer or bilayer), an oligolamellar vesicle (comprised of about two or about three monolayers or bilayers) or a multilamellar vesicle (comprised of more than about three monolayers or bilayers). Similarly, the vesicles prepared from proteins or polymers may comprise one or more concentric walls or membranes. The walls or membranes of vesicles prepared from proteins or polymers may be substantially solid (uniform), or they may be porous or semi-pourous. In certain preferred embodiments, the vesicles contain no sulfhydryl groups or disulfide linkages. The vesicles described herein include such entities commonly referred to as, for example, liposomes, lipospheres, micelles, bubbles, microbubbles, microspheres, lipid-, polymer- and/or protein-coated bubbles, microbubbles and/or microspheres, microballoons, aerogels, clatirate bound vesicles, and the like. The internal void of the vesicles may be filled with or encapsulate a liquid (including, for example, an aqueous liquid or an oil), a gas, a gaseous precursor, and/or a solid or solute material, including, for example, a targeting ligand and/or a bioactive agent, as desired.

“Liposome” refers to a generally spherical cluster or aggregate of amphipathic compounds, including lipid compounds, typically in the form of one or more concentric layers, for example, monolayers and/or bilayers. They may also be referred to herein as lipid vesicles. The liposomes may be formulated, for example, from ionic lipids and/or non-ionic lipids. Liposomes which are formulated from non-ionic lipids may also be referred to as “niosomes.”

“Liposphere” refers to an entity comprising a liquid or solid oil surrounded by one or more walls or membranes.

“Micelle” refers to colloidal entities formulated from lipids. In certain preferred embodiments, the micelles comprise a monolayer or hexagonal H2 phase configuration. In other preferred embodiments, the micelles may comprise a bilayer configuration.

“Aerogel” refers to generally spherical entities which are characterized by a plurality of small internal voids. The aerogels may be formulated from synthetic materials (for example, a foam prepared from baking resorcinol and formaldehyde), as well as natural materials, such as polysaccharides or proteins.

“Clathrate” refers to a solid, semi-porous or porous particle which may be associated with vesicles. In preferred form, the clathrates may form a cage-like structure containing cavities which comprise the vesicles. One or more vesicles may be bound to the clathrate. A stabilizing material may, if desired, be associated with the clathrate to promote the association of the vesicle with the clathrate. Suitable materials from which clathrates may be formulated include, for example, porous apatites, such as calcium hydroxyapatite, and precipitates of polymers and metal ions, such as alginic acid precipitated with calcium salts.

The vesicles of the present invention preferably contain a gas or gaseous precursor. “Gas filled vesicle” refers to vesicles in which there is encapsulated a gas. “Gaseous precursor filled vesicle” refers to vesicles in which there is encapsulated a gaseous precursor. The vesicles may be minimally, partially or substantially completely filled with the gas and/or gaseous precursor. In certain preferred embodiments, the vesicles may be substantially or completely filled with the gas and/or gaseous precursor. The term “substantially”, as used in reference to the gas and/or gaseous precursor filled vesicles, means that greater than about 50% of the internal void volume of the vesicle consists of a gas. Preferably, greater than about 60% of the internal void of the substantially filled vesicles consists of a gas, with greater than about 70% being more preferred. Even more preferably, greater than about 80% of the internal void of the substantially filled vesicles consists of a gas, with greater than about 90% being still more preferred. In particularly preferred embodiments, greater than about 95% of the internal void of the vesicles consists of a gas, with about 100% being especially preferred. Although not considered a preferred embodiment of the present invention, the vesicles may also contain, if desired, no or substantially no gas or gaseous precursor.

“Vesicle composition” refers to a composition, typically in an aqueous medium, which comprises vesicles.

“Vesicle formulation” refers to a vesicle composition which also comprises a bioactive agent. Suitable vesicles or vesicle species for use in vesicle formulations include, for example, gas filled vesicles and gaseous precursor filled vesicles.

“Emulsion” refers to a lipoidal mixture of two or more liquids and is generally in the form of a colloid. The lipids may be heterogeneously dispersed throughout the emulsion. Alternatively, the lipids may be aggregated in the form of, for example, clusters or layers, including mono- or bilayers.

“Suspension” refers to a mixture of finely divided liquid or solid particles floating in a liquid which can remain stable for extended periods of time.

“Hexagonal H II phase structure” refers to a generally tubular aggregation of lipids in liquid media, for example, aqueous media, in which the hydrophilic portion(s) of the lipids generally face inwardly in association with a liquid environment inside the tube. The hydrophobic portion(s) of the lipids generally radiate outwardly and the complex assumes the shape of a hexagonal tube. A plurality of tubes is generally packed together in the hexagonal phase structure.

“Patient” refers to animals, including mammals, preferably humans.

The phrases “internal region of a patient” and “region of interest” refer to the entire patient or to a particular area or portion of the patient. Internal regions of a patient and regions of interest may include, for example, areas being imaged with diagnostic imaging and/or areas being treated with a bioactive agent. Exemplary of such areas include, for example, the heart region, including myocardial tissue, as well as other bodily tissues, including the vasculature and circulatory system and cancerous tissue. The phrase “vasculature,” as used herein, denotes the blood vessels in the body or in an organ or part of the body.

“Bioactive agent” refers to a substance which may be used in connection with an application that is therapeutic or diagnostic in nature, such as in methods for diagnosing the presence or absence of a disease in a patient and/or in methods for the treatment of disease in a patient. As used herein, “bioactive agent” refers also to substances which are capable of exerting a biological effect iiz vitro and/or in vivo. The bioactive agents may be neutral or positively or negatively charged. Examples of suitable bioactive agents include diagnostic agents, pharmaceuticals, drugs, synthetic organic molecules, proteins, peptides, vitamins, steroids and genetic material, including nucleosides, nucleotides and polynucleotides.

“Diagnostic agent” refers to any agent which may be used in connection with methods for imaging an internal region of a patient and/or diagnosing the presence or absence of a disease in a patient. Exemplary diagnostic agents include, for example, contrast agents for use in connection with ultrasound, magnetic resonance imaging or computed tomography of a patient including, for example, the lipid and/or vesicle compositions described herein.

“Polymer”, as used herein, refers to molecules formed from the chemical union of two or more repeating units. Accordingly, included within the term “polymer” may be, for example, dimers, trimers and oligomers. The polymer may be synthetic, naturally-occurring or semisynthetic. In preferred form, the term “polymer” refers to molecules which comprise 10 or more repeating units. In certain preferred embodiments, the polymers which may be incorporated in the compositions described herein contain no sulfhydryl groups or disulfide linkages.

“Thickening agent” refers to any of a variety of generally hydrophilic materials which, when incorporated in the lipid and/or vesicle compositions described herein, may act as viscosity modifying agents, emulsifying and/or solubilizing agents, suspending agents, and tonicity raising agents. It is contemplated that the thickening agents may be capable of aiding in maintaining the stability of the compositions due to such properties.

“Dispersing agent” refers to a surface-active agent which, when added to a suspending medium of colloidal particles, including, for example, certain of the lipid and/or vesicle compositions described herein, may promote uniform separation of particles. In certain preferred embodiments, the dispersing agent may comprise a polymeric siloxane compound.

“Genetic material” refers generally to nucleotides and polynucleotides, including deoxyribonucleic acid (DNA) and ribonucleic acid (RNA). The genetic material may be made by synthetic chemical methodology known to one of ordinary skill in the art, or by the use of recombinant technology, or by a combination of the two. The DNA and RNA may optionally comprise unnatural nucleotides and may be single or double stranded. “Genetic material” refers also to sense and anti-sense DNA and RNA, that is, a nucleotide sequence which is complementary to a specific sequence of nucleotides in DNA and/or RNA.

“Pharmaceutical” or “drug” refers to any therapeutic or prophylactic agent which may be used in the treatment (including the prevention, diagnosis, alleviation, or cure) of a malady, affliction, disease or injury in a patient. Therapeutically useful peptides, polypeptides and polynucleotides may be included within the meaning of the term pharmaceutical or drug.

“Stabilizing material” refers to any material which is capable of improving the stability of compositions as described herein including, for example, emulsions, suspensions, dipersions and vesicle compositions. The improved stability involves, for example, the maintenance of a relatively balanced condition, and may be exemplified, for example, by increased resistance against destruction, decomposition, degradation and the like. In the case of preferred embodiments involving vesicles, especially gas filled vesicles, the stabilizing compounds may serve to improve the stability of the vesicles, for example, by minimizing or substantially (including completely) preventing the escape of gas entrapped within vesicles which may occur, for example, from rupture and/or coalescence of vesicles. The term “substantially”, as used in reference to the prevention of the escape of entrapped gas, means that greater than about 50% of the gas is maintained entrapped. Preferably, greater than about 60% of the gas is maintained entrapped, with greater than about 70% being more preferred. Even more preferably, greater than about 80% of the gas is maintained entrapped, with greater than about 90% being still more preferred. In particularly preferred embodiments, greater than about 95% of the gas is maintained entrapped. If desired, the gas may be completely maintained entrapped (i.e., about 100% of the gas is maintained entrapped). The stabilizing compounds may comprise discrete, individual compounds (monomers), or may comprise polymers. In the case of preferred embodiments involving lipids, the stabilizing materials may be associated covalently and/or non-covalently with the lipid compounds. Broadly spealing, the stabilizing compounds may comprise, for example, surfactants, film-forming materials, membranes and/or membrane forming materials. Exemplary stabilizing compounds which may be employed in the methods and compositions of the present invention include lipids, proteins and polymers. Encompassed also in the definition of “stabilizing material” are certain of the present bioactive agents. The stabilizing material may be neutral or positively or negatively charged. Preferred among the neutral stabilizing materials are polar materials. In certain embodiments, the stabilizing compounds may be substantially (including completely) crosslinked. The terms “crosslink”, crosslinked” and “crosslinking”, as used herein, generally refers to the linking of two or more stabilizing compounds, including lipid, protein and polymer stabilizing compounds, by one or more bridges. The bridges, which may be composed of one or more elements, groups or compounds, generally serve to join an atom from a first stabilizing compound molecule to an atom of a second stabilizing molecule. The crosslink bridges may involve covalent and/or non-covalent associations. Any of a variety of elements, groups and/or compounds may form the bridges in the crosslinks, and the stabilizing compounds may be crosslinked naturally or through synthetic means. For example, crosslinking may occur in nature in materials formulated from peptide chains which are joined by disulfide bonds of cystine residues, as in keratins, insulin, and other proteins. Alternatively, crosslinking may be effected by suitable chemical modification, such as, for example, by combining a compound, such as a stabilizing material, and a chemical substance that may serve as a crosslinking agent, which are caused to react, for example, by exposure to heat, high-energy radiation, ultrasonic radiation, and the like. Examples include, for example, crosslinking with sulfur which may be present, for example, as sulfhydryl groups in cysteine residues, to provide disulfide linkages, crosslinking with organic peroxides, crosslinking of unsaturated materials by means of high-energy radiation, crosslinking with dimethylol carbamate, and the like. The term “substantially”, as used in reference to crosslinked stabilizing compounds, means that greater than about 50% of the stabilizing compounds contain crosslinking bridges. In certain embodiments, preferably greater than about 60% of the crosslinked stabilizing compounds contain crosslinking bridges, with greater than about 70% being more preferred. Even more preferably, greater than about 80% of the crosslinked stabilizing compounds contain crosslinking bridges, with greater than about 90% being still more preferred. In certain particularly preferred embodiments, greater than about 95% of the crosslinked stabilizing compounds contain crosslinking bridges. If desired, the substantially crosslinked stabilizing compounds may be completely crosslinked (i.e., about 100% of the crosslinked stabilizing compounds contain crosslinking bridges). In the most preferred embodiments, the stabilizing compounds may be substantially (including completely) non-crosslinked. The term “substantially”, as used in reference to non-crosslinked stabilizing compounds, means that greater than about 50% of the stabilizing compounds are devoid of crosslinking bridges. Preferably, greater than about 60% of the stabilizing compounds are devoid of crosslinking bridges, with greater than about 70% being more preferred. Even more preferably, greater than about 80% of the stabilizing compounds are devoid of crosslinking bridges, with greater than about 90% being still more preferred. In particularly preferred embodiments, greater than about 95% of the stabilizing compounds are devoid of crosslinking bridges. If desired, the substantially non-crosslinked stabilizing compounds may be completely non-crosslinked (i.e., about 100% of the stabilizing compounds are devoid of crosslinking bridges).

“Vesicle stability” refers to the ability of gas-filled vesicles to retain the gas entrapped therein after being exposed, for about one minute, to a pressure of about 300 mm Hg. Vesicle stability is measured in percent (%), this being the fraction of the amount of gas which is originally entrapped in the vesicle and which is retained after release of the pressure. Vesicle stability includes reference also to “vesicle resilience” which refers to the ability of a vesicle to return to its original size after release of the pressure.

“Covalent association” refers to an intermolecular association or bond which involves the sharing of electrons in the bonding orbitals of two atoms.

“Non-covalent association” refers to intermolecular interaction among two or more separate molecules which does not involve a covalent bond. Intermolecular interaction is dependent upon a variety of factors, including, for example, the polarity of the involved molecules, the charge (positive or negative), if any, of the involved molecules, and the like. Non-covalent associations are preferably selected from the group consisting of ionic interaction, dipole-ipole interaction and van der Waal's forces and combinations thereof.

“Ionic interaction” or “electrostatic interaction” refers to intermolecular interaction among two or more molecules, each of which is positively or negatively charged. Thus, for example, “ionic interaction” or “electrostatic interaction” refers to the attraction between a first, positively charged molecule and a second, negatively charged molecule. Exemplary ionic or electrostatic interactions include, for example, the attraction between a negatively charged stabilizing material, for example, genetic material, and a positively charged lipid, for example, a cationic lipid, such as lauryltrimethylammonium bromide.

“Dipole-dipole interaction” refers generally to the attraction which can occur among two or more polar molecules. Thus, “dipole-dipole interaction” refers to the attraction of the uncharged, partial positive end of a first polar molecule, commonly designated as δ⁺, to the uncharged, partial negative end of a second polar molecule, commonly designated as δ⁻. Dipole-dipole interactions are exemplified, for example, by the attraction between the electropositive head group, for example, the choline head group, of phosphatidylcholine and an electronegative atom, for example, a heteroatom, such as oxygen, nitrogen or sulphur, which is present in a stabilizing material, such as a polysaccharide. “Dipole-dipole interaction” refers also to intermolecular hydrogen bonding in which a hydrogen atom serves as a bridge between electronegative atoms on separate molecules and in which a hydrogen atom is held to a first molecule by a covalent bond and to a second molecule by electrostatic forces.

“Van der Waal's forces” refers to the attractive forces between non-polar molecules that are accounted for by quantum mechanics. Van der Waal's forces are generally associated with momentary dipole moments which are induced by neighboring molecules and which involve changes in electron distribution.

“Hydrogen bond” refers to an attractive force, or bridge, which may occur between a hydrogen atom which is bonded covalently to an electronegative atom, for example, oxygen, sulfur, nitrogen, and the like, and another electronegative atom. The hydrogen bond may occur between a hydrogen atom in a first molecule and an electronegative atom in a second molecule (intermolecular hydrogen bonding). Also, the hydrogen bond may occur between a hydrogen atom and an electronegative atom which are both contained in a single molecule (intramolecular hydrogen bonding).

“Hydrophilic interaction” refers to molecules or portions of molecules which may substantially bind with, absorb and/or dissolve in water. This may result in swelling and/or the formation of reversible gels.

“Hydrophobic interaction” refers to molecules or portions of molecules which do not substantially bind with, absorb and/or dissolve in water.

“Biocompatible” refers to materials which are generally not injurious to biological functions and which will not result in any degree of unacceptable toxicity, including allergenic responses and disease states.

“In combination with” refers, in certain embodiments, to the incorporation of a targeting ligand in a composition of the present invention, including lipid compositions and vesicle compositions. “In combination with” may refer also to the incorporation of a bioactive agent in a composition of the present invention, including lipid compositions and vesicle compositions. The bioactive agent and/or targeting ligand may be combined with the present compositions in any of a variety of ways. If desired, the bioactive agent and/or targeting ligand may be associated covalently with one or more components of the present compositions such as, for example, lipid compounds, proteins, polymers and/or vesicles or other optional stabilizing materials. Also, if desired, there may be substantially no covalent association of the bioactive agent and/or targeting ligand with the other components of the present compositions such as, for example, the lipid compounds, proteins, polymers and/or vesicles or other optional stabilizing materials. The term “substantially no”, as used herein in reference to the lack of covalent association of bioactive agent and/or targeting ligand with other components of the compositions such as, for example, lipid compounds, proteins, polymers and/or vesicles, may mean that less than about 50% such as, for example, from from about 0% to less than about 50% (and all specific percentages and combinations and subcombinations of ranges of percentages therein) of the bioactive agent and/or targeting ligand may be covalently associated with other components of the compositions. Preferably, less than about 40% of the bioactive agent and/or targeting ligand may be covalently associated with other components of the compositions, with less than about 30% being more preferred. Even more preferably, less than about 20% of the bioactive agent and/or targeting ligand may be covalently associated with other components of the compositions, with less than about 10% being yet more preferred. In still more preferred embodiments, there may be completely no (i.e., 0%) covalent association of the bioactive agent and/or targeting ligand with other components of the compositions.

Bioactive agent and/or targeting ligand which have substantially no covalent association with other components of the compositions may sometimes be referred to herein as “unbound” or “free” bioactive agent and/or targeting ligand. In such compositions, the bioactive agent and/or targeting ligand may, if desired, be associated non-covalently with other components of the compositions such as, for example, the lipid compounds, proteins, polymers and/or vesicles or other optional stabilizing materials. In addition, if desired, there may be substantially no non-covalent association of the unbound or free bioactive agent and/or targeting ligand with other components of the compositions including, for example, lipid compounds, proteins, polymers and/or vesicles. The term “substantially no”, as used herein in reference to the lack of non-covalent association of bioactive agent and/or targeting ligand with other components of the compositions such as, for example, lipid compounds, proteins, polymers and/or vesicles, may mean that less than about 50% such as, for example, from about 0% to less than about 50% (and all specific percentages and combinations and subcombinations of ranges of percentages therein) of the unbound or free bioactive agent and/or targeting ligand may be associated non-covalently with other components of the compositions. Preferably, less than about 40% of the unbound or free bioactive agent and/or targeting ligand may be associated non-covalently with other components of the compositions, with less than about 30% being more preferred. Even more preferably, less than about 20% of the unbound or free bioactive agent and/or targeting ligand may be associated non-covalently with other components of the compositions, with less than about 10% being yet more preferred. In still more preferred embodiments, there may be completely no (i.e., 0%) non-covalent association of the unbound or free bioactive agent and/or targeting ligand with other components of the compositions.

In the case of vesicle compositions, the bioactive agent and/or targeting ligand may be entrapped within the internal void of the vesicle. The bioactive agent and/or targeting ligand may also be integrated within the layer(s) or wall(s) of the vesicle, for example, by being interspersed among lipids which are contained within the vesicle layer(s) or wall(s). In addition, it is contemplated that the bioactive agent and/or targeting ligand may be located on the surface of a vesicle. In any case, the bioactive agent and/or targeting ligand may interact chemically with the walls of the vesicles, including, for example, the inner and/or outer surfaces of the vesicle and may remain substantially adhered thereto. Such interaction may take the form of, for example, covalent association or non-covalent association. In certain embodiments, the interaction may result in the stabilization of the vesicle.

“Targeting ligand” refers to any material or substance which may promote targeting of tissues and/or receptors in vivo with the compositions of the present invention. The targeting ligand may be synthetic, semi-synthetic, or naturally-occurring. The targeting ligands of the present invention are advantageously capable of targeting vascular plaques.

A “precursor” to a targeting ligand refers to any material or substance which may be converted to a targeting ligand. Such conversion may involve, for example, anchoring a precursor to a targeting ligand.

“Phosphorylated serine moiety” refers to a compound radical which contains a serine group and an oxidized phosphorus group, as discussed in greater detail herein.

“Peptide” refers to a nitrogenous compound which may contain from about 2 to about 100 amino acid residues. In certain preferred embodiments, the peptides which may be incorporated in the compositions described herein contain no sulfhydryl groups or disulfide linkages.

“Protein” refers to a nitrogenous compound which may contain more than about 100 amino acid residues. In certain preferred embodiments, the proteins which may be incorporated in the compositions described herein contain no sulfhydryl groups or disulfide linkages.

“Coat” or “coating” refers to the interaction of the stabilizing material with the lipid and/or vesicles and may involve covalent and/or non-covalent association.

“Tissue” refers generally to specialized cells which may perform a particular function. It should be understood that the term “tissue,” as used herein, may refer to an individual cell or a plurality or aggregate of cells, for example, membranes or organs. The term “tissue” also includes reference to an abnormal cell or a plurality of abnormal cells. Exemplary tissues include, for example, myocardial tissue (also referred to as heart tissue or myocardium), including myocardial cells and cardiomyocites, membranous tissues, including endothelium and epithelium, laminae, connective tissue, including interstitial tissue, and tumors.

“Receptor” refers to a molecular structure within a cell or on the surface of the cell which is generally characterized by the selective binding of a specific substance. Exemplary receptors include, for example, cell-surface receptors for peptide hormones, neurotransmitters, antigens, complement fragments, and immunoglobulins and cytoplasmic receptors for steroid hormones.

“Endothelial cells” or “endothelium” refers to an aggregate of cells and/or tissue which may be normal and/or diseased and which may comprise a single layer of flattened transparent endothelial cells that may be joined edge to edge or in an overlapping fashion to form a membrane. Endothelial cells are found on the free surfaces of the serous membranes, as part of the lining membrane of the heart, blood vessels, and lymphatics, on the surface of the brain and spinal cord, and in the anterior chamber of the eye. Endothelium originates from the embryonic mesoblast and includes heart tissue, including infarcted heart tissue, cardiovasculature, the peripheral vasculature, such as arteries, veins, and capillaries (the location of which is noted as peripheral to the heart), blood clots and the region surrounding atherosclerotic plaque.

“Epithelial cells” or “epithelium” refers to an aggregate of cells and/or tissue which may be normal and/or diseased and which may comprise one or more layers of cells that may be united together by an interstitial cementitious substance supported on a basement-membrane. Epithelium may be classified into various classes, including, for example, a single layer of cells (simple epithelium); more than a single layer of cells (stratified epithelium); and about three or four layers of cells that are fitted together substantially without the appearance of stratification. The different forms of simple epithelium are usually referred to as squamous, pavement, columnar, glandular, spheroidal and/or ciliated. Epithelium originates from the embryonic epiblast or hypoblast. Epithelium includes heart tissue, including infarcted heart tissue, cardiovasculature, the peripheral vasculature, such as arteries, veins, and capillaries, blood clots and the region surrounding atherosclerotic plaque.

“Myocardial” refers generally to heart tissue, including cardiomyocite, myocardial, endocardial and epicardial cells. The term “myocardial” includes reference to infarcted heart tissue, the cardiovasculature, the peripheral vasculature, such as arteries, veins, and capillaries (the location of which is noted as peripheral to the heart), blood clots, thrombi, and the region surrounding atherosclerotic plaque.

“Cardiac region” refers generally to the heart and surrounding tissues, structures and blood vessels, including the coronary arteries.

“Tumor cells” or “tumor” refers to an aggregate of abnormal cells and/or tissue which may be associated with diseased states that are characterized by uncontrolled cell proliferation. The disease states may involve a variety of cell types, including, for example, endothelial, epithelial and myocardial cells. Included among the disease states are neoplasms, cancer, leukemia and restenosis injuries.

“Vascular plaque” refers to a generally fibrous, elevated area of intimal thickening in arterial walls, and is a highly characteristic lesion of advancing atherosclerosis. Plaques typically comprise a central core of extracellular lipid (with cholesterol crystals) and necrotic cell debris (also referred to as “gruel”) covered by a fibromuscular layer or cap containing large numbers of smooth muscle cells, macrophages, and collagen.

The present invention is directed, in part, to methods and compositions for diagnostic imaging. As discussed more fully hereinafter, the methods and compositions of the present invention are particularly suitable for use in connection with the diagnosis and/or treatment of patients suffering from vascular plaque. The present invention provides, in part, methods and compositions which are advantageously adapted to target such plaques in vivo. This targeting enables the use of the compositions described herein, for example, in diagnostic imaging methods, including ultrasound imaging methods, which may thereby permit a physician to identify and/or confirm the presence of the plaque, as well as the level of risk posed to the patient by the plaque.

In accordance with the present invention, there are provided embodiments involving lipid and/or vesicle compositions. Embodiments are provided which comprise lipid compositions comprising a lipid, a targeting ligand which may target tissues, cells and/or receptors in vivo, a gas or gaseous precursor, and optionally, an oil. Embodiments are also provided herein which comprise vesicle compositions comprising, in an aqueous carrier, vesicles formulated from lipids or polymers, a targeting ligand which may target tissues, cells and/or receptors in vivo, a gas or gaseous precursor, and optionally, an oil. In connection with lipid compositions, and especially lipid compositions in the form of vesicle compositions, it may be advantageous to prepare the lipid compositions at a temperature below the gel to liquid crystalline phase transition temperature of the involved lipids. This phase transition temperature is the temperature at which a lipid bilayer will convert from a gel state to a liquid crystalline state. See, for example, Chapman et al., J. Biol. Chem. 1974 249, 2512-2521.

It is generally believed that vesicles which are prepared from lipids that possess higher gel state to liquid crystalline state phase transition temperatures tend to have enhanced impermeability at any given temperature. See Derek Marsh, CRC Handbook of Lipid Bilayers (CRC Press, Boca Raton, Fla. 1990), at p. 139 for main chain melting transitions of saturated diacyl-sn-glycero-3-phosphocholines. The gel state to liquid crystalline state phase transition temperatures of various lipids will be readily apparent to those skilled in the art and are described, for example, in Gregoriadis, ed., Liposome Technology, Vol. I, 1-18 (CRC Press, 1984). The following table lists some of the representative lipids and their phase transition temperatures.

TABLE 1
Saturated Diacyl-sn-Glycero-3-Phosphocholines:
Main Chain Melting Transition Temperatures

| Number of Carbons | Main Phase Transition |
|---|---|
| in Acyl Chains | Temperature (° C.) |
| 1,2-(12:0) | −1.0 |
| 1,2-(13:0) | 13.7 |
| 1,2-(14:0) | 23.5 |
| 1,2-(15:0) | 34.5 |
| 1,2-(16:0) | 41.4 |
| 1,2-(17:0) | 48.2 |
| 1,2-(18:0) | 55.1 |
| 1,2-(19:0) | 61.8 |
| 1,2-(20:0) | 64.5 |
| 1,2-(21:0) | 71.1 |
| 1,2-(22:0) | 74.0 |
| 1,2-(23:0) | 79.5 |
| 1,2-(24:0) | 80.1 |

See, for example, Derek Marsh, CRC Handbook of Lipid Bilayers, p. 139 (CRC Press, Boca Raton, Fla. 1990).

It may be possible to enhance the stability of vesicles by incorporating in the present lipid and/or vesicle compositions at least a minor amount, for example, about 1 to about 10 mole percent, based on the total amount of lipid employed, of a negatively charged lipid. Suitable negatively charged lipids include, for example, phosphatidylserine, phosphatidic acid, and fatty acids. Without intending to be bound by any theory or theories of operation, it is contemplated that such negatively charged lipids provide added stability by counteracting the tendency of vesicles to rupture by fusing together. Thus, the negatively charged lipids may act to establish a uniform negatively charged layer on the outer surface of the vesicle, which will be repulsed by a similarly charged outer layer on other vesicles which are proximate thereto. In this way, the vesicles may be less prone to come into touching proximity with each other, which may lead to a rupture of the membrane or skin of the respective vesicles and consolidation of the contacting vesicles into a single, larger vesicle. A continuation of this process of consolidation will, of course, lead to significant degradation of the vesicles.

The lipid materials used, especially in connection with vesicle compositions, are also preferably flexible. This means, in the context of the present invention, that the vesicles can alter their shape, for example, to pass through an opening having a diameter that is smaller than the diameter of the vesicle.

A wide variety of lipids are believed to be suitable for incorporation in the lipid compositions. With particular reference to vesicle compositions, for example, micelles and/or liposomes, any of the materials or combinations thereof which are known to those skilled in the art as suitable for their preparation may be used. The lipids used may be of either natural, synthetic or semi-synthetic origin. As noted above, suitable lipids generally include, for example, fatty acids, neutral fats, phosphatides, glycolipids, aliphatic alcohols and waxes, terpenes, sesquiterpenes, and steroids.

Exemplary lipids which may be used to prepare the present lipid compositions included, for example, fatty acids, lysolipids, including lysophospholipids, phosphocholines, such as those associated with platelet activation factors (PAF) (Avanti Polar Lipids, Alabaster, Ala.), including l-alkyl-2-acetoyl-sn-glycero 3-phosphocholines, and 1-alkyl-2-hydroxy-sn-glycero 3-phosphocholines, which target blood clots; phosphatidylcholine with both saturated and unsaturated lipids, including dioleoylphosphatidylcholine; dimyristoylphosphatidylcholine; dipentadecanoyl-phosphatidylcholine; dilauroylphosphatidylcholine; dipalmitoylphosphatidylcholine (DPPC); distearoylphosphatidylcholine (DSPC); and diarachidonylphosphatidylcholine (DAPC); phosphatidylethanolamines, such as dioleoylphosphatidylethanolamine, dipalmitoylphosphatidylethanolamine (DPPE) and distearoylphosphatidylethanolamine (DSPE); phosphatidylserines; phosphatidylglycerols, including distearoylphosphatidyl-glycerol (DSPG) and dipalmitoyl-glycerolsuccinate (DPGS); phosphatidylinositol; sphingolipids such as sphingomyelin; sphingosines; glycolipids such as ganglioside GM1 and GM2; glucolipids; sulfatides; glycosphingolipids; phosphatidic acids, such as dipalmitoylphosphatidic acid (DPPA) and distearoylphosphatidic acid (DSPA); palmitic acid; stearic acid; arachidonic acid; oleic acid; lipids bearing polymers, such as chitin, hyaluronic acid, polyvinylpyrrolidone or polyethylene glycol (PEG), also referred to herein as “pegylated lipids”, with preferred lipids bearing polymers including DPPE-PEG (DPPE-PEG), which refers to the lipid DPPE having a PEG polymer attached thereto, including, for example, DPPE-PEG5000, which refers to DPPE having attached thereto a PEG polymer having a mean average molecular weight of about 5000; lipids bearing sulfonated mono-, di-, oligo- or polysaccharides; steroids, such as for example, cholesterol, cholesterol sulfate, cholesterol hemisuccinate and cholesterol amines; tocopherol hemisuccinate; lipids with ether and ester-linked fatty acids; polymerized lipids (a wide variety of which are well known in the art); diacetyl phosphate; dicetyl phosphate; stearylamine; cardiolipin; phospholipids with short chain fatty acids of about 6 to about 8 carbons in length; synthetic phospholipids with asymmetric acyl chains, such as, for example, one acyl chain of about 6 carbons and another acyl chain of about 12 carbons; ceramides; non-ionic liposomes including niosomes such as polyoxyalkylene (e.g., polyoxyethylene) fatty acid esters, polyoxyalkylene (e.g., polyoxyethylene) fatty alcohols, polyoxyalkylene (e.g., polyoxyethylene) fatty alcohol ethers, polyoxyalkylene (e.g., polyoxyethylene) sorbitan fatty acid esters (such as, for example, the class of compounds referred to as TWEEN, including, for example, TWEEN® 20, TWEEN® 40 and TWEEN® 80, commercially available from ICI Americas, Inc., Wilmington, Del.), glycerol polyethylene glycol oxystearate, glycerol polyethylene glycol ricinoleate, alkyloxylated (e.g., ethoxylated) soybean sterols, alkyloxylated (e.g., ethoxylated) castor oil, polyoxyethylene-polyoxypropylene polymers, and polyoxyalkylene (e.g., polyoxyethylene) fatty acid stearates; sterol aliphatic acid esters including cholesterol sulfate, cholesterol butyrate, cholesterol isobutyrate, cholesterol palmitate, cholesterol stearate, lanosterol acetate, ergosterol palmitate, and phytosterol n-butyrate; sterol esters of sugar acids including cholesterol glucuronide, lanosterol glucuronide, 7-dehydro-cholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; long chain alcohols including n-decyl alcohol, lauryl alcohol, myristyl alcohol, cetyl alcohol, and n-octadecyl alcohol; 6-(5-cholesten-3β-yloxy)-1-thio-β-D-galactopyranoside; digalactosyldiglyceride; 6-(5-cholesten-3β-yloxy)-hexyl-6-amino-6-deoxy-1-thio-β-D-galactopyranoside; 6-(5-cholesten-3β-yloxy)hexyl-6-amino-6-deoxyl-1-thioa-D-mannopyranoside; 12-(((7′-diethylamino-coumarin-3-yl)-carbonyl)methylamino)-octadecanoic acid; N-[12-(((7′-diethylamino-coumarin-3-yl)-carbonyl)methylamino)-octadecanoyl]-2-aminopalmitic acid; cholesteryl(4′-trimethyl-ammonio)butanoate; N-succinyldioleoylphosphatidylethanolamine; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine and palmitoylhomocysteine, and/or any combinations thereof. In preferred embodiments, the stabilizing materials comprise phospholipids, including one or more of DPPC, DPPE, DPPA, DSPC, DSPE, DSPG, and DAPC.

Examples of suitable fluorinated lipids include but are not limited to compounds of the formula:
CₙF₂₊₁(CH₂)ₘC(O)OOP(OO⁻)O(CH₂)_{w} N⁺(CH₃)₃CₙF₂ₙ₊₁(CH₂)ₘC(O)O
wherein m is 0 to about 18, n is 1 to about 12; and w is 1 to about 8. Examples of and methods for the synthesis of these, as well as other fluorinated lipids useful in the present invention, are set forth in Unger, U.S. Pat. No. 5,997,898, Reiss et al. U.S. Pat. No. 5,344,930, Frezard, F., et al., Biochem Biophys Acta, 1192:61-70 (1994), and Frezard, F., et al., Art. Cells Blood Subs and Immob Biotech., 22:1403-1408 (1994), the disclosures of each of which are incorporated herein by reference in their entirety. One specific example of a difluoroacyl glycerylphosphatidyl-choline, nonafluorinated diacyl glycerylphosphatidylcholine, is represented by compound A, below. One skilled in the art will appreciate that analogous fluorinated derivatives of other common phospholipids (diacylphosphatidyl serine, diacylphosphatidyl ethanolamine, diacylphosphatidyl glycerol, diacylphosphatidyl glycerol, etc.) as well as fluorinated derivatives of fatty acyl esters and free fatty acids may also function in accordance with the scope of the invention. Additionally lipid based and fluorinated (including perfluorinated) surfactants may be used as stabilizing materials in the present invention.

Examples of polymerized lipids include unsaturated lipophilic chains such as alkenyl or alkynyl, containing up to about 50 carbon atoms. Further examples are phospholipids such as phosphoglycerides and sphingolipids carrying polymerizable groups; and saturated and unsaturated fatty acid derivatives with hydroxyl groups, such as for example triglycerides of d-12-hydroxyoleic acid, including castor oil and ergot oil.

Polymerization may be designed to include hydrophilic substituents such as carboxyl or hydroxyl groups, to enhance dispersability so that the backbone residue resulting from biodegradation is water soluble. Suitable polymerizable lipids are also described, for example, by Klaveness et al, U.S. Pat. No. 5,536,490, the disclosure of which is hereby incorporated by reference herein in its entirety.

Exemplary polymerizable and/or fluorinated lipid compounds which may be utilized in the compositions of the present invention are illustrated below. 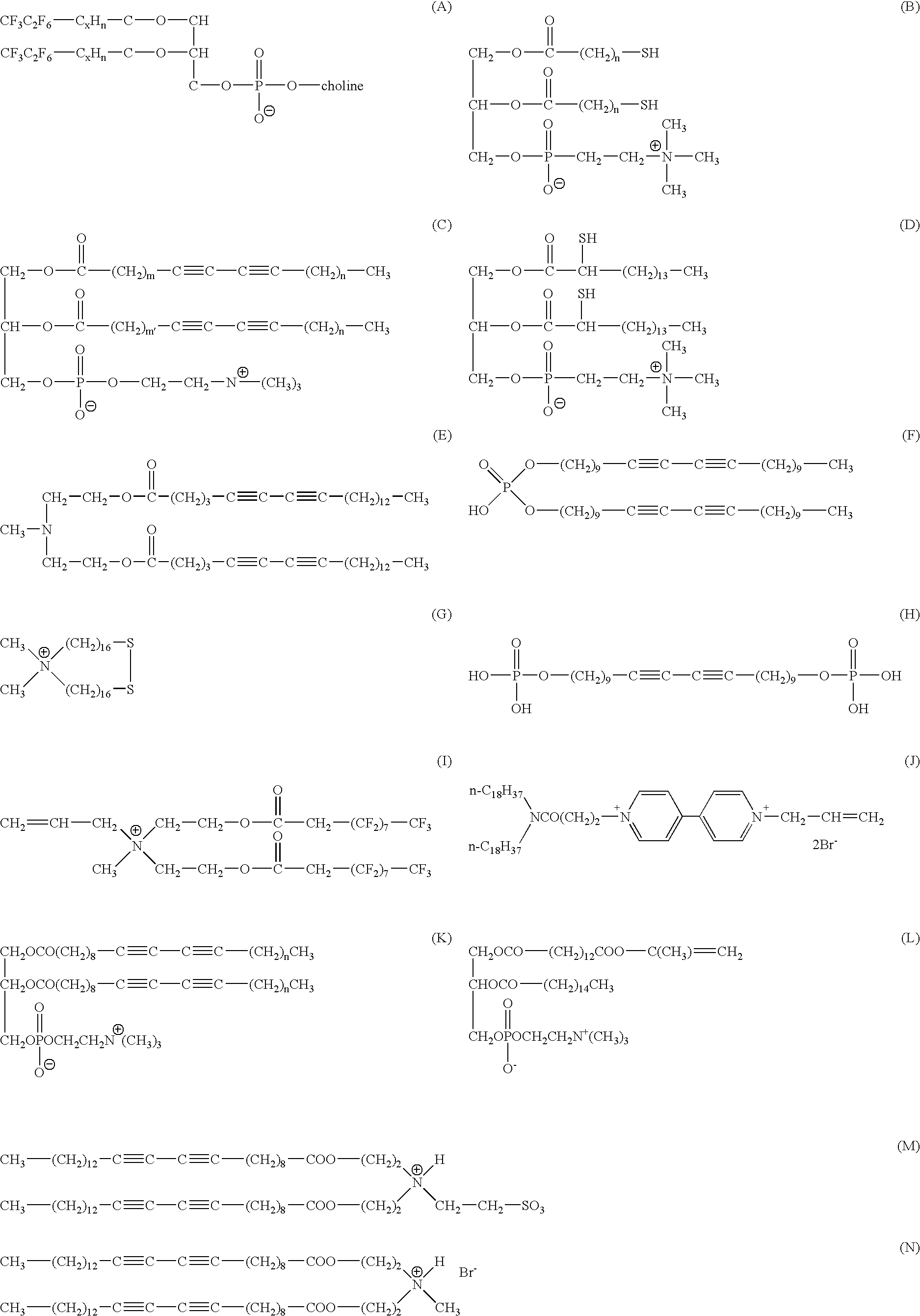 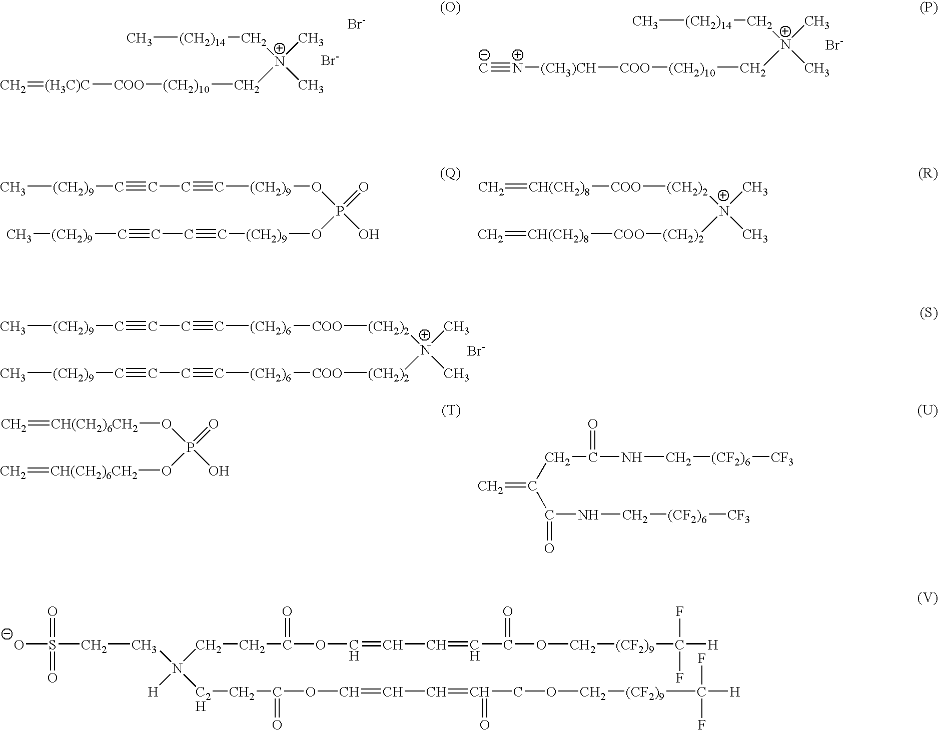
In formula A, above, x is an integer from about 8 to about 18, and n is 2x. Most preferably x is 12 and n is 24. In formulas B, C and K above, m, n, m′ and n′ are, independently, an integer of from about 8 to about 18, preferably about 10 to about 14.

Other lipids which may be employed in the present compositions include, for example, 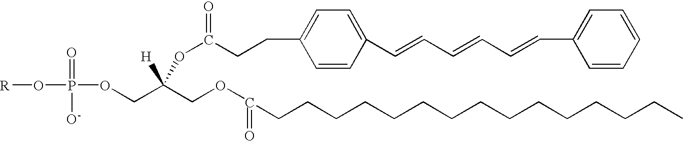
where R is choline, 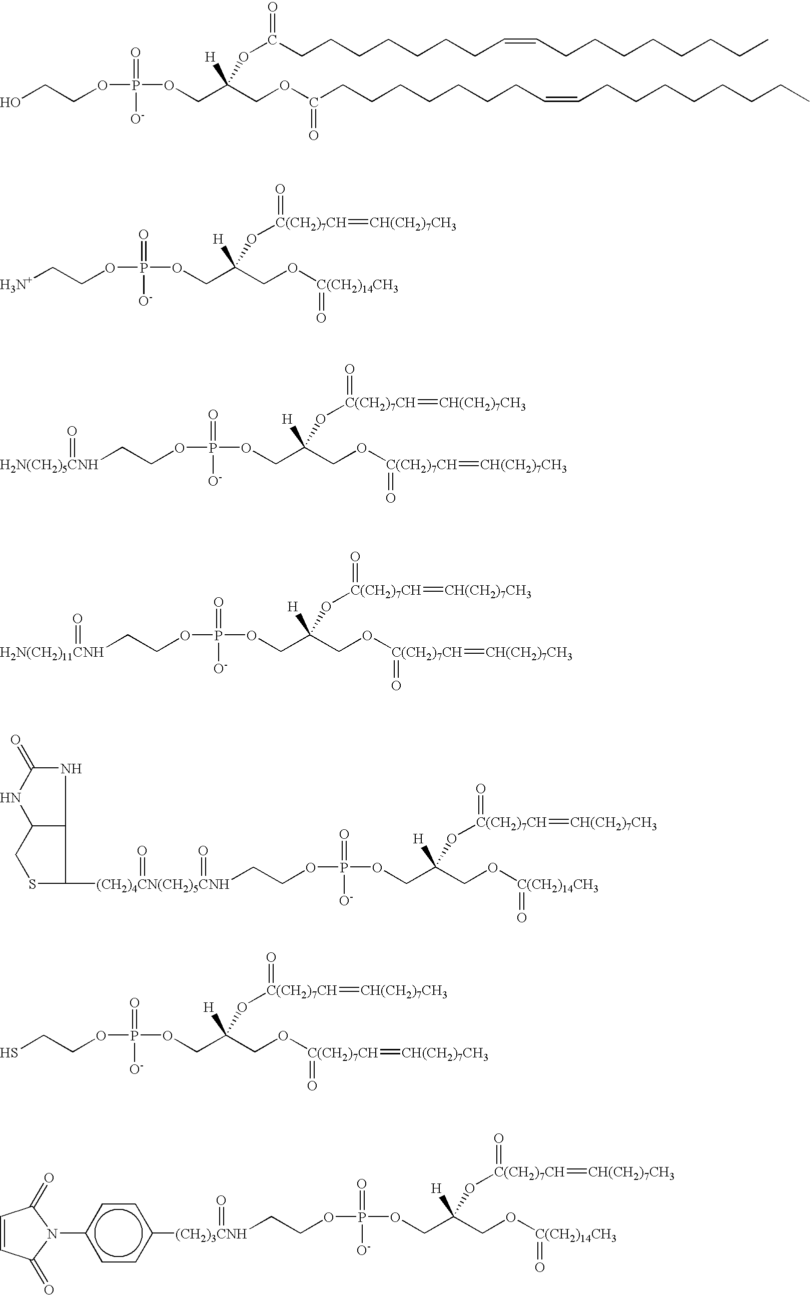 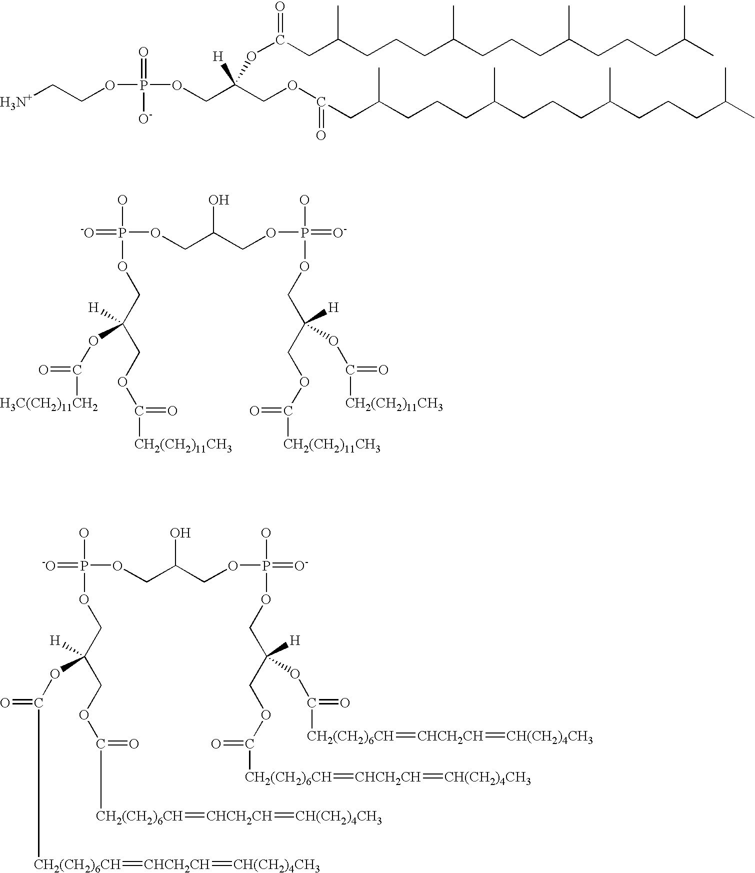
deuterated lipids, such as

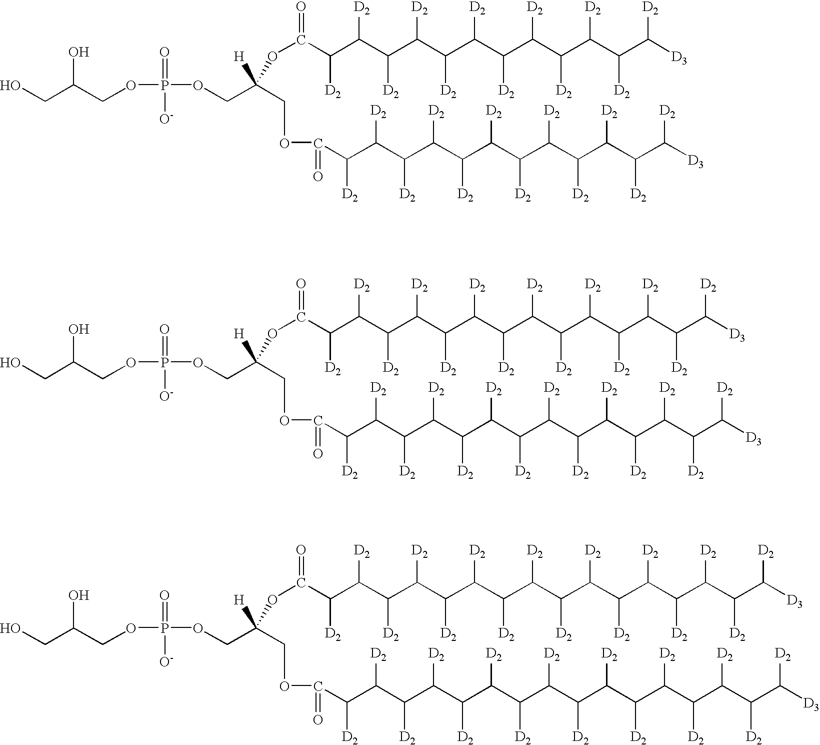

If desired, a cationic lipid may be used, such as, for example, N-[1-(2,3-dioleoyloxy)propyl]-N,N,N-trimethylammonium chloride (DOTMA), 1,2-dioleoyloxy-3-(trimethylammonio)propane (DOTAP); and 1,2-dioleoyl-3-(4′-trimethylammonio)-butanoyl-sn-glycerol (DOTB). If a cationic lipid is employed in the lipid compositions, the molar ratio of cationic lipid to non-cationic lipid may be, for example, from about 1:1000 to about 1:100. Preferably, the molar ratio of cationic lipid to non-cationic lipid may be from about 1:2 to about 1:10, with a ratio of from about 1:1 to about 1:2.5 being preferred. Even more preferably, the molar ratio of cationic lipid to non-cationic lipid may be about 1:1.

In the case of lipid compositions which contain both cationic and non-cationic lipids, a wide variety of lipids may be employed as the non-cationic lipid. Preferably, this non-cationic lipid comprises one or more of DPPC, DPPE and dioleoylphosphatidylethanolamine. In lieu of the cationic lipids listed above, lipids bearing cationic polymers, such as polylysine or polyarginine, as well as alkyl phosphonates, alkyl phosphinates, and alkyl phosphites, may also be used in the lipid compositions. The present compositions may also include one or more of the cationic lipid compounds set forth in U.S. Pat. No. 5,830,430, the disclosures of which are hereby incorporated by reference herein, in their entirety.

In certain preferred embodiments, the lipid compositions comprise phospholipids, particularly one or more of DPPC, DPPE, DPPA, DSPC, DSPE, DSPG, and DAPC (20 carbons).

In addition, saturated and unsaturated fatty acids may be employed in the present lipid compositions may include molecules that preferably contain from about 12 carbons to about 22 carbons, in linear or branched form. Hydrocarbon groups consisting of isoprenoid units and/or prenyl groups can be used as well. Examples of saturated fatty acids that are suitable include, for example, lauric, myristic, palmitic, and stearic acids. Suitable unsaturated fatty acids that may be used include, for example, lauroleic, physeteric, myristoleic, palmitoleic, petroselinic, and oleic acids. Examples of branched fatty acids that may be used include, for example, isolauric, isomyristic, isopalmitic, and isostearic acids. Other lipids which may be employed in the present compositions include those disclosed in Unger et al., U.S. Pat. No. 6,090,800 and Unger, U.S. Pat. No. 6,028,066, the disclosures of which are hereby incorporated herein by reference, in their entireties.

In addition to lipid compositions and/or vesicle compositions formulated from lipids, embodiments of the present invention may also involve vesicles formulated from polymers which may be of natural, semi-synthetic (modified natural) or synthetic origin. As used herein, the term polymer denotes a compound comprised of two or more repeating monomeric units, and preferably 10 or more repeating monomeric units. The phrase semi-synthetic polymer (or modified natural polymer), as employed herein, denotes a natural polymer that has been chemically modified in some fashion. Exemplary natural polymers suitable for use in the present invention include naturally occurring polysaccharides. Such polysaccharides include, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrin, dextrose, polydextrose, pustulan, chitin, agarose, keratan, chondroitan, dermatan, hyaluronic acid, alginic acid, xanthan gum, starch and various other natural homopolymer or heteropolymers, such as those containing one or more of the following aldoses, ketoses, acids or amines: erythrose, threose, ribose, arabinose, xylose, lyxose, allose, altrose, glucose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Accordingly, suitable polymers include, for example, proteins, such as albumin. Exemplary semi-synthetic polymers include carboxymethylcellulose, hydroxymethylcellulose, hydroxypropylmethylcellulose, methylcellulose, and methoxycellulose. Exemplary synthetic polymers suitable for use in the present invention include polyethylenes (such as, for example, polyethylene glycol, polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbons, fluorinated carbons (such as, for example, polytetrafluoroethylene), and polymethylmethacrylate, and derivatives thereof. Preferred are biocompatible synthetic polymers or copolymers prepared from monomers, such as acrylic acid, methacrylic acid, ethyleneimine, crotonic acid, acrylamide, ethyl acrylate, methyl methacrylate, 2-hydroxyethyl methacrylate (HEMA), lactic acid, glycolic acid, ε-caprolactone, acrolein, cyanoacrylate, bisphenol A, epichlorhydrin, hydroxyalkyl-acrylates, siloxane, dimethylsiloxane, ethylene oxide, ethylene glycol, hydroxyalkyl-methacrylates, N-substituted acrylamides, N-substituted methacrylamides, N-vinyl-2-pyrrolidone, 2,4-pentadiene-1-ol, vinyl acetate, acrylonitrile, styrene, p-amino-styrene, p-amino-benzyl-styrene, sodium styrene sulfonate, sodium 2-sulfoxyethylmethacrylate, vinyl pyridine, aminoethyl methacrylates, 2-methacryloyloxy-trimethylammonium chloride, and polyvinylidene, as well polyfunctional crosslinking monomers such as N,N-methylenebisacrylamide, ethylene glycol dimethacrylates, 2,2′-(p-phenylenedioxy)-diethyl dimethacrylate, divinylbenzene, triallylamine and methylenebis-(4-phenyl-isocyanate), including combinations thereof. Preferable polymers include polyacrylic acid, polyethyleneimine, polymethacrylic acid, polymethylmethacrylate, polysiloxane, polydimethylsiloxane, polylactic acid, poly(ε-caprolactone), epoxy resin, poly(ethylene oxide), poly(ethylene glycol), and polyamide (nylon) polymers. Preferable copolymers include the following: polyvinylidene-polyacrylonitrile, polyvinylidene-polyacrylonitrile-polymethylmethacrylate, polystyrene-polyacrylonitrile and poly d-1, lactide co-glycolide polymers. A preferred copolymer is polyvinylidene-polyacrylonitrile. Other suitable biocompatible monomers and polymers will be readily apparent to those skilled in the art, once armed with the present disclosure.

As noted above, the present lipid compositions also preferably comprise a gas or gaseous precursor. The gas may desirably provide the lipid compositions with enhanced reflectivity, particularly in connection with vesicle compositions in which the gas is entrapped within the vesicles. This may increase their effectiveness as contrast agents.

Preferred gases are gases which are inert and which are biocompatible, that is, gases which are not injurious to biological function. Preferred gases include those selected from the group consisting of air, noble gases, such as helium, rubidium hyperpolarized xenon, hyperpolarized argon, hyperpolarized helium, neon, argon and xenon, carbon dioxide, nitrogen, fluorine, oxygen, sulfur-based gases, such as sulfur hexafluoride and sulfur tetrafluoride, fluorinated gases, including, for example, partially fluorinated gases or completely fluorinated gases. Exemplary fluorinated gases include the fluorocarbon gases, such as the perfluorocarbon gases, and mixtures thereof. Paramagnetic gases, such as ¹⁷O₂ may also be used in the lipid compositions.

In preferred embodiments, the gas comprises a fluorinated gas. Such fluorinated gases include materials which contain one, or more than one, fluorine atom. Preferred are gases which contain more than one fluorine atom, with perfluorocarbons (that is, fully fluorinated fluorocarbons) being more preferred. Preferably, the perfluorocarbon gas is selected from the group consisting of perfluoromethane, perfluoroethane, perfluoropropane, perfluorobutane, perfluorocyclobutane and mixtures thereof. More preferably, the perfluorocarbon gas is perfluoropropane or perfluorobutane, with perfluorobutane being particularly preferred. Another preferable gas is sulfur hexafluoride. Yet another preferable gas is heptafluoropropane, including 1,1,1,2,3,3,3-heptafluoropropane and its isomer, 1,1,2,2,3,3,3-heptafluoropropane. It is contemplated that mixtures of different types of gases, such as mixtures of a perfluorocarbon gas and another type of gas, such as air, can also be used in the compositions of the present invention. Other gases, including the gases exemplified above, would be readily apparent to one skilled in the art based on the present disclosure.

In certain preferred embodiments, a gas, for example, air or a perfluorocarbon gas, is combined with a liquid perfluorocarbon, such as perfluoropentane, perfluorohexane, perfluoroheptane, perfluorooctane, perfluorononane, perfluorooctyl-bromide (PFOB), perfluorodecalin, perfluorododecalin, perfluorooctyliodide, perfluorotripropylamine and perfluorotributylamine.

It may also be desirable to incorporate in the lipid compositions a precursor to a gaseous substance. Such precursors include materials that are capable of being converted to a gas in vivo. Preferably, the gaseous precursor is biocompatible, and the gas produced in vivo is biocompatible also.

Among the gaseous precursors which are suitable for use in compositions described herein are agents which are sensitive to pH. These agents include materials that are capable of evolving gas, for example, upon being exposed to a pH that is neutral or acidic. Examples of such pH sensitive agents include salts of an acid which is selected from the group consisting of inorganic acids, organic acids and mixtures thereof. Carbonic acid (H₂CO₃) is an example of a suitable inorganic acid, and aminomalonic acid is an example of a suitable organic acid. Other acids, including inorganic and organic acids, would be readily apparent to one skilled in the art based on the present disclosure.

Gaseous precursors which are derived form salts are preferably selected from the group consisting of alkali metal salts, ammonium salts and mixtures thereof. More preferably, the salt is selected from the group consisting of carbonate, bicarbonate, sesquecarbonate, aminomalonate and mixtures thereof.

Examples of suitable gaseous precursor materials which are derived from salts include, for example, lithium carbonate, sodium carbonate, potassium carbonate, lithium bicarbonate, sodium bicarbonate, potassium bicarbonate, magnesium carbonate, calcium carbonate, magnesium bicarbonate, ammonium carbonate, ammonium bicarbonate, ammonium sesquecarbonate, sodium sesquecarbonate, sodium aminomalonate and ammonium aminomalonate. Aminomalonate is well known in the art, and its preparation is described, for example, in Thanassi, Biochemistry, Vol. 9, no. 3, pp. 525-532 (1970); Fitzpatrick et al., Inorganic Chemistry, Vol. 13, no. 3 pp. 568-574 (1974); and Stelmashok et al., Koordinatsionnaya Khimiya, Vol. 3, no. 4, pp. 524-527 (1977). The disclosures of these publications are hereby incorporated herein by reference.

In addition to, or instead of, being sensitive to changes in pH, the gaseous precursor materials may also comprise compounds which are sensitive to changes in temperature. Exemplary of suitable gaseous precursors which are sensitive to changes in temperature are the perfluorocarbons. As the artisan will appreciate, a particular perfluorocarbon may exist in the liquid state when the lipid compositions are first made, and are thus used as a gaseous precursor. Alternatively, the perfluorocarbon may exist in the gaseous state when the lipid compositions are made, and are thus used directly as a gas. Whether the perfluorocarbon is used as a liquid or a gas generally depends on its liquid/gas phase transition temperature, or boiling point. For example, a preferred perfluorocarbon, perfluoropentane, has a liquid/gas phase transition temperature (boiling point) of 29.5° C. This means that perfluoropentane is generally a liquid at room temperature (about 25° C.), but is converted to a gas within the human body, the normal temperature of which is about 37° C., which is above the transition temperature of perfluoropentane. Thus, under normal circumstances, perfluoropentane is a gaseous precursor. As a further example, there are the homologs of perfluoropentane, namely perfluorobutane and perfluorohexane. The liquid/gas transition of perfluorobutane is 4° C. and that of perfluorohexane is 57° C. Thus, perfluorobutane can be useful as a gaseous precursor, although more likely as a gas, whereas perfluorohexane can be useful as a gaseous precursor because of its relatively high boiling point. As known to one of ordinary skill in the art, the effective boiling point of a substance may be related to the pressure to which that substance is exposed. This relationship is exemplified by the ideal gas law: PV=nRT, where P is pressure, V is volume, n is moles of substance, R is the gas constant, and T is temperature. The ideal gas law indicates that as pressure increases, the effective boiling point increases also. Conversely, as pressure decreases, the effective boiling point decreases.

A wide variety of materials can be used as gaseous precursors in the present compositions. It is only required that the material be capable of undergoing a phase transition to the gas phase upon passing through the appropriate temperature. Suitable gaseous precursors include, for example, hexafluoroacetone, isopropyl acetylene, allene, tetrafluoroallene, boron trifluoride, 1,2-butadiene, 2,3-butadiene, 1,3-butadiene, 1,2,3-trichloro-2-fluoro-1,3-butadiene, 2-methyl-1,3-butadiene, hexafluoro-1,3-butadiene, butadiyne, 1-fluorobutane, 2-methylbutane, perfluorobutane, 1-butene, 2-butene, 2-methyl-1-butene, 3-methyl-1-butene, perfluoro-1-butene, perfluoro-2-butene, 4-phenyl-3-butene-2-one, 2-methyl-1-butene-3-yne, butyl nitrate, 1-butyne, 2-butyne, 2-chloro-1,1,1,4,4,4-hexafluorobutyne, 3-methyl-1-butyne, perfluoro-2-butyne, 2-bromo-butyraldehyde, carbonyl sulfide, crotononitrile, cyclobutane, methylcyclobutane, octafluorocyclobutane, perfluorocyclobutene, 3-chlorocyclopentene, perfluorocyclo-pentane, octafluorocyclo-pentene, cyclopropane, perfluorocyclopropane, 1,2-dimethyl-cyclopropane, 1,1-dimethyl-cyclopropane, 1,2-dimethylcyclopropane, ethylcyclopropane, methylcyclopropane, diacetylene, 3-ethyl-3-methyl diaziridine, 1,1,1-trifluorodiazoethane, dimethyl amine, hexafluorodimethylamine, dimethylethylamine, bis(dimethylphosphine)-amine, perfluorohexane, perfluoroheptane, perfluorooctane, 2,3-dimethyl-2-norbornane, perfluorodimethylamine, dimethyloxonium chloride, 1,3-dioxolane-2-one, 4-methyl-1,1,1,2-tetrafluoroethane, 1,1,1-trifluoroethane, 1,1,2,2-tetrafluoroethane, 1,1,2-trichloro-1,2,2-trifluoroethane, 1,1-dichloroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1,1-dichloro-2-fluoroethane, 1-chloro-1,1,2,2-tetrafluoroethane, 2-chloro-1,1-difluoroethane, chloroethane, chloropentafluoroethane, dichlorotrifluoroethane, fluoroethane, perfluoroethane, nitropentafluoroethane, nitrosopentafluoroethane, perfluoroethylamine, ethyl vinyl ether, 1,1-dichloroethane, 1,1-dichloro-1,2-difluoroethane, 1,2-difluoroethane, methane, trifluoromethanesulfonylchloride, trifluoromethanesulfonylfluoride, bromodifluoronitrosomethane, bromofluoromethane, bromochlorofluoromethane, bromotrifluoromethane, chlorodifluoronitromethane, chlorodinitromethane, chlorofluoromethane, chlorotrifluoromethane, chlorodifluoromethane, dibromodifluoromethane, dichlorodifluoromethane, dichlorofluoromethane, difluoromethane, difluoroiodomethane, disilanomethane, fluoromethane, iodomethane, iodotrifluoromethane, nitrotrifluoromethane, nitrosotrifluoromethane, tetrafluoromethane, trichlorofluoromethane, trifluoromethane, 2-methylbutane, methyl ether, methyl isopropyl ether, methyllactate, methylnitrite, methylsufide, methyl vinyl ether, neopentane, nitrous oxide, 1,2,3-nonadecane-tricarboxylic acid 2-hydroxytrimethyl ester, 1-nonene-3-yne, 1,4-pentadiene, n-pentane, perfluoropentane, 4-amino-4-methylpentan-2-one, 1-pentene, 2-pentene (cis and trans), 3-bromopent-1-ene, perfluoropent-1-ene, tetrachlorophthalic acid, 2,3,6-trimethyl-piperidine, propane, 1,1,1,2,2,3-hexafluoropropane, 1,2-epoxypropane, 2,2-difluoro-propane, 2-aminopropane, 2-chloropropane, heptafluoro-1-nitropropane, heptafluoro-1-nitrosopropane, perfluoropropane, propene, hexafluoropropane, 1,1,1,2,3,3-hexafluoro-2,3-dichloropropane, 1-chloropropane, chloropropane-(trans), 2-chloropropane, 3-fluoropropane, propyne, 3,3,3-trifluoropropyne, 3-fluorostyrene, sulfur (di)-decafluoride (S₂F₁₀), 2,4-diaminotoluene, trifluoroacetonitrile, trifluoromethyl peroxide, trifluoromethyl sulfide, tungsten hexafluoride, vinyl acetylene and vinyl ether.

Perfluorocarbons are both preferred gases and preferred gaseous precursors for use in connection with the compositions employed in the methods of the present invention. Included among such perfluorocarbons are saturated perfluorocarbons, unsaturated perfluorocarbons, and cyclic perfluorocarbons. Saturated perfluorocarbons, which are usually preferred, have the formula CₙF₂ₙ₊₂. In preferred embodiments, the gases or gaseous precursors are perfluorocarbons having from 1 to about 12 carbon atoms (and all combinations and subcombinations of ranges therein), preferably from about 2 to about 10 carbons, more preferably from about 3 to about 8 carbons, and even more preferably from about 3 to about 6 carbons. Suitable perfluorocarbons include, for example, perfluoromethane, perfluoroethane, perfluoropropane, perfluorobutane, perfluorocyclobutane, perfluoropentane, perfluorohexane, perfluoroheptane, perfluorooctane, perfluorononane, perfluorodecane, perfluorodecalin, perfluoroundecane and perfluorododecane and mixtures thereof. Preferably, the perfluorocarbon is selected from the group consisting of perfluoropropane, perfluorobutane, perfluorocyclobutane, perfluoropentane, perfluorohexane and perfluorooctane, with perfluorobutane being particularly preferred. Cyclic perfluorocarbons, which have the formula CₐF₂ₙ, where n is from about 3 to about 8, preferably from about 3 to about 6, may also be preferred, and include, for example, hexafluorocyclopropane, octafluorocyclobutane, and decafluorocyclopentane.

In addition to the perfluorocarbons, it may be desirable to utilize stable fluorocarbons which are not completely fluorinated. Such fluorocarbons include heptafluoropropane, for example, 1,1,1,2,3,3,3-heptafluoropropane and its isomer, 1,1,2,2,3,3,3-heptafluoropropane.

The gaseous precursor materials may be also photoactivated materials, such as diazonium ion and aminomalonate. As discussed more fully hereinafter, certain lipid and/or vesicle compositions, and particularly vesicle compositions, may be formulated so that gas is formed at the target tissue or by the action of sound on the lipid composition. Examples of gaseous precursors are described, for example, in U.S. Pat. Nos. 5,088,499 and 5,149,319, the disclosures of which are hereby incorporated herein by reference, in their entirety. Other gaseous precursors, in addition to those exemplified above, will be apparent to one skilled in the art based on the present disclosure.

The gaseous substances and/or gaseous precursors are preferably incorporated in the lipid and/or vesicle compositions irrespective of the physical nature of the composition. Thus, it is contemplated that the gaseous substances and/or precursors thereto may be incorporated, for example, in lipid compositions in which the lipids are aggregated randomly, as well as in vesicle compositions, including vesicle compositions which are formulated from lipids, such as micelles and liposomes. Incorporation of the gaseous substances and/or precursors thereto in the lipid and/or vesicle compositions may be achieved by using any of a number of methods. For example, in the case of vesicles based on lipids, the formation of gas filled vesicles can be achieved by shaking or otherwise agitating an aqueous mixture which comprises a gas or gaseous precursor and one or more lipids. This promotes the formation of stabilized vesicles within which the gas or gas precursor is encapsulated.

In addition, a gas may be bubbled directly into an aqueous mixture of lipid and/or vesicle-forming compounds. Alternatively, a gas instillation method can be used as disclosed, for example, in U.S. Pat. Nos. 5,352,435 and 5,228,446, the disclosures of which are hereby incorporated herein by reference, in their entirety. Suitable methods for incorporating the gas or gas precursor in cationic lipid compositions are disclosed also in U.S. Pat. No. 4,865,836, the disclosures of which are hereby incorporated herein by reference. Other methods would be apparent to one skilled in the art based on the present disclosure. Preferably, the gas may be instilled in the lipid and/or vesicle compositions after or during the addition of the stabilizing material and/or during formation of vesicles.

In preferred embodiments, the gaseous substances and/or gaseous precursor materials are incorporated in vesicle compositions, with micelles and liposomes being preferred. As discussed in detail below, vesicles in which a gas or gaseous precursor or both are encapsulated are advantageous in that they provide improved reflectivity in vivo.

As discussed more fully hereinafter, it is preferred that the lipid compositions, and especially the vesicle compositions, be formulated from lipids and optional stabilizing compounds to promote the formation of stable vesicles. In addition, it is also preferred that the lipid and/or vesicle compositions comprise a highly stable gas as well. The phrase “highly stable gas” refers to a gas which has limited solubility and diffusability in aqueous media Exemplary highly stable gases include perfluorocarbons since they are generally less diffusible and relatively insoluble in aqueous media. Accordingly, their use may promote the formation of highly stable vesicles.

In certain embodiments, it may be desirable to use a fluorinated compound, especially a perfluorocarbon compound, which may be in the liquid state at the temperature of use of the lipid and/or vesicle compositions, including, for example, the in vivo temperature of the human body, to assist or enhance the stability of the lipid and/or vesicle compositions, and especially, the gas filled vesicles. Suitable fluorinated compounds include, for example, fluorinated surfactants, such as fluorinated surfactants which are commercially available as ZONYL® surfactants (the DuPont Company, Wilmington, Del.), as well as liquid perfluorocarbons, such as for example, perfluorooctylbromide (PFOB), perfluorodecalin, perfluorododecalin, perfluorooctyliodide, perfluorotripropylamine, and perfluorotributylamine. In general, perfluorocarbons comprising about six or more carbon atoms will be liquids at normal human body temperature. Among these perfluorocarbons, perfluorooctylbromide and perfluorohexane, which are liquids at room temperature, are preferred. The gas which is present may be, for example, nitrogen or perfluoropropane, or may be derived from a gaseous precursor, which may also be a perfluorocarbon, for example, perfluoropentane. In the latter case, the lipid and/or vesicle compositions may be prepared from a mixture of perfluorocarbons, which for the examples given, would be perfluoropropane (gas) or perfluoropentane (gaseous precursor) and perfluorooctylbromide (liquid). Although not intending to be bound by any theory or theories of operation, it is believed that, in the case of vesicle compositions, the liquid fluorinated compound may be situated at the interface between the gas and the membrane or wall surface of the vesicle. There may be thus formed a further stabilizing layer of liquid fluorinated compound on the internal surface of the stabilizing compound, for example, a biocompatible lipid used to form the vesicle, and this perfluorocarbon layer may also prevent the gas from diffusing through the vesicle membrane. A gaseous precursor, within the context of the present invention, is a liquid at the temperature of manufacture and/or storage, but becomes a gas at least at or during the time of use.

Thus, it has been discovered that a liquid fluorinated compound, such as a perfluorocarbon, when combined with a gas or gaseous precursor ordinarily used to make the lipid and/or vesicle compositions described herein, may confer an added degree of stability not otherwise obtainable with the gas or gaseous precursor alone. Thus, it is within the scope of the present invention to utilize a gas or gaseous precursor, such as a perfluorocarbon gaseous precursor, for example, perfluoropentane, together with a perfluorocarbon which remains liquid after administration to a patient, that is, whose liquid to gas phase transition temperature is above the body temperature of the patient, for example, perfluorooctylbromide. Perfluorinated surfactants, such as ZONYL® fluorinated surfactants, may be used to stabilize the lipid and/or vesicle compositions, and to act, for example, as a coating for vesicles. Preferred perfluorinated surfactants are the partially fluorinated phosphocholine surfactants. In these preferred fluorinated surfactants, the dual alkyl compounds may be fluorinated at the terminal alkyl chains and the proximal carbons may be hydrogenated. These fluorinated phosphocholine surfactants may be used for making the targeted lipid and/or vesicle compositions of the present invention.

In connection with embodiments involving vesicle compositions, the size of the vesicles can be adjusted for the particular intended end use including, for example, diagnostic and/or therapeutic use. The size of the vesicles may preferably range from about 30 nanometers (nm) to about 100 micrometers (μm) in diameter, and all combinations and subcombinations of ranges therein. More preferably, the vesicles have diameters of from about 100 nm to about 10 μm, with diameters of from about 200 nm to about 7 μm being even more preferred. In connection with particular uses, for example, intravascular use, including magnetic resonance imaging of the vasculature, it may be preferred that the vesicles be no larger that about 30 μm in diameter, with smaller vesicles being preferred, for example, vesicles of no larger than about 12 μm in diameter. In certain preferred embodiments, the diameter of the vesicles may be about 7 μm or less, with vesicles having a mean diameter of about 5 μm or less being more preferred, and vesicles having a mean diameter of about 3 μm or less being even more preferred. It is contemplated that these smaller vesicles may perfuse small vascular channels, such as the microvasculature, while at the same time providing enough space or room within the vascular channel to permit red blood cells to slide past the vesicles.

The size of the gas filled vesicles can be adjusted, if desired, by a variety of procedures including, for example, shaking, microemulsification, vortexing, extrusion, filtration, sonication, homogenization, repeated freezing and thawing cycles, extrusion under pressure through pores of defined size, and similar methods.

As noted above, compositions employed herein may also include, with respect to their preparation, formation and use, gaseous precursors that can be activated to change from a liquid or solid state into a gas by temperature, pH, light, and energy (such as ultrasound). The gaseous precursors may be made into gas by storing the precursors at reduced pressure. For example, a vial stored under reduced pressure may create a headspace of perfluoropentane or perfluorohexane gas, useful for creating a preformed gas prior to injection. Preferably, the gaseous precursors may be activated by temperature. Set forth below is a table listing a series of gaseous precursors which undergo phase transitions from liquid to gaseous states at relatively close to normal body temperature (37° C.) or below, and the size of the emulsified droplets that would be required to form a vesicle of a maximum size of 10 μm.

TABLE 2
Physical Characteristics of Gaseous Precursors and
Diameter of Emulsified Droplet to Form a 10 μm Vesicle*

| | | | | Diameter (μm) of |
|---|---|---|---|---|
| | | | | emulsified droplet |
| | Molecular | Boiling Point | | to make 10 |
| Compound | Weight | (° C.) | Density | micron vesicle |
| perfluoro | 288.04 | 28.5 | 1.7326 | 2.9 |
| pentane | | | | |
| 1- | 76.11 | 32.5 | 0.67789 | 1.2 |
| fluorobutane | | | | |
| 2-methyl | 72.15 | 27.8 | 0.6201 | 2.6 |
| butane | | | | |
| (isopentane) | | | | |
| 2-methyl 1- | 70.13 | 31.2 | 0.6504 | 2.5 |
| butene | | | | |
| 2-methyl-2- | 70.13 | 38.6 | 0.6623 | 2.5 |
| butene | | | | |
| 1-butene-3- | 66.10 | 34.0 | 0.6801 | 2.4 |
| yne-2-methyl | | | | |
| 3-methyl-1- | 68.12 | 29.5 | 0.6660 | 2.5 |
| butyne | | | | |
| octafluoro | 200.04 | −5.8 | 1.48 | 2.8 |
| cyclobutane | | | | |
| decafluoro | 238.04 | −2 | 1.517 | 3.0 |
| butane | | | | |
| hexafluoro | 138.01 | −78.1 | 1.607 | 2.7 |
| ethane | | | | |

*Source: Chemical Rubber Company Handbook of Chemistry and Physics, Robert C. Weast and David R. Lide, eds., CRC Press, Inc. Boca Raton, Florida (1989-1990).

The perfluorocarbons, as already indicated, are preferred for use as the gas or gaseous precursors, as well as additional stabilizing components.

As noted above, it is preferred to optimize the utility of the lipid and/or vesicle compositions, especially vesicle compositions formulated from lipids, by using gases of limited solubility. The phrase “limited solubility” refers to the ability of the gas to diffuse out of the vesicles by virtue of its solubility in the surrounding aqueous medium. A greater solubility in the aqueous medium imposes a gradient with the gas in the vesicle such that the gas may have a tendency to diffuse out of the vesicle. A lesser solubility in the aqueous milieu, may, on the other hand, decrease or eliminate the gradient between the vesicle and the interface such that diffusion of the gas out of the vesicle may be impeded. Preferably, the gas entrapped in the vesicle has a solubility less than that of oxygen, that is, about 1 part gas in about 32 parts water. See Matheson Gas Data Book, 1966, Matheson Company Inc. More preferably, the gas entrapped in the vesicle possesses a solubility in water less than that of air; and even more preferably, the gas entrapped in the vesicle possesses a solubility in water less than that of nitrogen.

It may be desirable, in certain embodiments, to formulate vesicles from polymeric materials, including substantially impermeable polymeric materials. In these embodiments, it is generally unnecessary to employ a gas which is highly insoluble also. For example, stable vesicle compositions which comprise substantially impermeable polymeric materials may be formulated with gases having higher solubilities, for example, air or nitrogen.

In addition to, or instead of, the lipid and/or polymeric compounds discussed above, the compositions described herein may comprise one or more stabilizing materials. Exemplary of such stabilizing materials are, for example, biocompatible polymers. The stabilizing materials may be employed to desirably assist in the formation of vesicles and/or to assure substantial encapsulation of the gases or gaseous precursors. Even for relatively insoluble, non-diffusible gases, such as perflubropropane or sulfur hexafluoride, improved vesicle compositions may be obtained when one or more stabilizing materials are utilized in the formation of the gas and gaseous precursor filled vesicles. These compounds may help improve the stability and the integrity of the vesicles with regard to their size, shape and/or other attributes.

The terms “stable” or “stabilized”, as used herein, means that the vesicles may be substantially resistant to degradation, including, for example, loss of vesicle structure or encapsulated gas or gaseous precursor, for a useful period of time. Typically, the vesicles employed in the present invention have a desirable shelf life, often retaining at least about 90% by volume of its original structure for a period of at least about two to three weeks under normal ambient conditions. In preferred form, the vesicles are desirably stable for a period of time of at least about 1 month, more preferably at least about 2 months, even more preferably at least about 6 months, still more preferably about eighteen months, and yet more preferably up to about 3 years. The vesicles described herein, including gas and gaseous precursor filled vesicles, may also be stable even under adverse conditions, such as temperatures and pressures which are above or below those experienced under normal ambient conditions.

The stability of the vesicles described herein may be attributable, at least in part, to the materials from which the vesicles are made, including, for example, the lipids and/or polymers described above, and it is often not necessary to employ additional stabilizing materials, although it is optional and may be preferred to do so. Such additional stabilizing materials and their characteristics are described more fully hereinafter.

The materials from which the vesicles are constructed are preferably biocompatible lipid or polymer materials, and of these, the biocompatible lipids are preferred. In addition, because of the ease of formulation, including the capability of preparing vesicles immediately prior to administration, these vesicles may be conveniently made on site.

Biocompatible polymers useful as stabilizing materials for preparing the gas and gaseous precursor filled vesicles may be of natural, semi-synthetic (modified natural) or synthetic origin. As used herein, the term polymer denotes a compound comprised of two or more repeating monomeric units, and preferably 10 or more repeating monomeric units. The phrase semi-synthetic polymer (or modified natural polymer), as employed herein, denotes a natural polymer that has been chemically modified in some fashion. Exemplary natural polymers suitable for use in the present invention include naturally occurring polysaccharides. Such polysaccharides include, for example, arabinans, fructans, fucans, galactans, galacturonans, glucans, mammns, xylans (such as, for example, inulin), levan, fucoidan, carrageenan, galatocarolose, pectic acid, pectins, including amylose, pullulan, glycogen, amylopectin, cellulose, dextran, dextrin, dextrose, polydextrose, pustulan, chitin, agarose, keratan, chondroitan, dermatan, hyaluronic acid, alginic acid, xanthan gum, starch and various other natural homopolymer or heteropolymers, such as those containing one or more of the following aldoses, ketoses, acids or amines: erythrose, threose, ribose, arabinose, xylose, lyxose, allose, altrose, glucose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Accordingly, suitable polymers include, for example, proteins, such as albumin. Exemplary semi-synthetic polymers include carboxymethylcellulose, hydroxymethylcellulose, hydroxypropylmethylcellulose, methylcellulose, and methoxycellulose. Exemplary synthetic polymers suitable for use in the present invention include polyethylenes (such as, for example, polyethylene glycol, polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbons, fluorinated carbons (such as, for example, polytetrafluoroethylene), and polymethylmethacrylate, and derivatives thereof. Methods for the preparation of vesicles which employ polymers as stabilizing compounds will be readily apparent to those skilled in the art, once armed with the present disclosure, when the present disclosure is coupled with information known in the art, such as that described and referred to in Unger, U.S. Pat. No. 5,205,290, the disclosures of which are hereby incorporated herein by reference, in their entirety.

Particularly preferred embodiments of the present invention may involve vesicles which comprise three components: (1) a neutral lipid, for example, a nonionic or zwitterionic lipid, (2) a negatively charged lipid, and (3) a lipid bearing a stabilizing material, for example, a hydrophilic polymer. Preferably, the amount of the negatively charged lipid will be greater than about 1 mole percent of the total lipid present, and the amount of lipid bearing a hydrophilic polymer will be greater than about 1 mole percent of the total lipid present. Exemplary and preferred negatively charged lipids include phosphatidic acids. The lipid bearing a hydrophilic polymer will desirably be a lipid covalently linked to the polymer, and the polymer will preferably have a weight average molecular weight of from about 400 to about 100,000. Suitable hydrophilic polymers are preferably selected from the group consisting of polyethylene glycol (PEG), polypropylene glycol, polyvinylalcohol, and polyvinylpyrrolidone and copolymers thereof, with PEG polymers being preferred. Preferably, the PEG polymer has a molecular weight of from about 1000 to about 7500, with molecular weights of from about 2000 to about 5000 being more preferred. The PEG or other polymer may be bound to the lipid, for example, DPPE, through a covalent bond, such as an amide, carbamate or amine linkage. In addition, the PEG or other polymer may be linked to a targeting ligand, or other phospholipids, with a covalent bond including, for example, amide, ester, ether, thioester, thioamide or disulfide bonds. Where the hydrophilic polymer is PEG, a lipid bearing such a polymer will be said to be “pegylated.” In preferred form, the lipid bearing a hydrophilic polymer may be DPPE-PEG, including, for example, DPPE-PEG5000, which refers to DPPE having a polyethylene glycol polymer of a mean weight average molecular weight of about 5000 attached thereto (DPPE-PEG5000). Other suitable pegylated lipids include, for example, distearoylphosphatidylethanolamine-polyethylene glycol (DSPE-PEG), including DSPE-PEG5000, dipalmitoyl-glycero-succinate-polyethylene glycol (DPGS-PEG), stearyl-polyethylene glycol and cholesteryl-polyethylene glycol.

In certain preferred embodiments of the present invention, the lipid compositions may include about 77.5 mole % DPPC, 12.5 mole % of DPPA, and 10 mole % of DPPE-PEG5000. Also preferred are compositions which comprise about 80 to about 90 mole % DPPC, about 5 to about 15 mole % DPPA and about 5 to about 15 mole % DPPE-PEG5000. Especially preferred are compositions which comprise DPPC, DPPA and DPPE-PEG5000 in a mole % ratio of 82:10:8, respectively. DPPC is substantially neutral, since the phosphatidyl portion is negatively charged and the choline portion is positively charged. Consequently, DPPA, which is negatively charged, may be added to enhance stabilization in accordance with the mechanism described above. DPPE-PEG provides a pegylated material bound to the lipid membrane or skin of the vesicle by the DPPE moiety, with the PEG moiety free to surround the vesicle membrane or skin, and thereby form a physical barrier to various enzymatic and other endogenous agents in the body whose function is to degrade such foreign materials. The DPPE-PEG may provide more vesicles of a smaller size which are safe and stable to pressure when combined with other lipids, such as DPPC and DPPA, in the given ratios. It is also theorized that the pegylated material, because of its structural similarity to water, may be able to defeat the action of the macrophages of the human immune system, which would otherwise tend to surround and remove the foreign object. The result is an increase in the time during which the stabilized vesicles may function as diagnostic imaging contrast media.

The vesicle compositions may be prepared from other materials, in addition to the materials described above, provided that the vesicles so prepared meet the stability and other criteria set forth herein. These materials may be basic and fundamental, and form the primary basis for creating or establishing the stabilized gas and gaseous precursor filled vesicles. On the other hand, they may be auxiliary, and act as subsidiary or supplementary agents which can enhance the functioning of the basic stabilizing material or materials, or contribute some desired property in addition to that afforded by the basic stabilizing material.

However, it is not always possible to determine whether a given material is a basic or an auxiliary agent, since the functioning of the material in question is determined empirically, for example, by the results produced with respect to producing stabilized vesicles. As examples of how these basic and auxiliary materials may function, it has been observed that the simple combination of a biocompatible lipid and water or saline when shaken will often give a cloudy solution subsequent to autoclaving for sterilization. Such a cloudy solution may function as a contrast agent, but is aesthetically objectionable and may imply instability in the form of undissolved or undispersed lipid particles. Cloudy solutions may be also undesirable where the undissolved particulate matter has a diameter of greater than about 7 μm, and especially greater than about 10 μm. Manufacturing steps, such as sterile filtration, may also be problematic with solutions which contain undissolved particulate matter. Thus, propylene glycol may be added to remove this cloudiness by facilitating dispersion or dissolution of the lipid particles. The propylene glycol may also function as a wetting agent which can improve vesicle formation and stabilization by increasing the surface tension on the vesicle membrane or slin. It is possible that the propylene glycol can also function as an additional layer that may coat the membrane or skin of the vesicle, thus providing additional stabilization. As examples of such further basic or auxiliary stabilizing materials, there are conventional surfactants which may be used; see D'Arrigo U.S. Pat. Nos. 4,684,479 and 5,215,680.

Additional auxiliary and basic stabilizing materials include such agents as peanut oil, canola oil, olive oil, safflower oil, corn oil, or any other oil commonly known to be ingestible which is suitable for use as a stabilizing compound in accordance with the teachings herein. Various auxiliary and basic stabilizing materials are disclosed, for example, in Unger, U.S. Pat. No. 5,736,121, the disclosures of which are incorporated herein by reference, in their entirety.

In addition, compounds used to make mixed micelle systems may be suitable for use as basic or auxiliary stabilizing materials, and these include, for example, lauryltrimethylammonium bromide (dodecyl-), cetyltrimethylammonium bromide (hexadecyl-), myristyltrimethylammonium bromide (tetradecyl-), alkyldimethyl-benzylammonium chloride (where alkyl is C₁₂, C₁₄ or C₁₆,), benzyldimethyldodecyl-ammonium bromide/chloride, benzyldimethyl hexadecylammonium bromide/chloride, benzyldimethyl tetradecylammonium bromide/chloride, cetyldimethylethylammonium bromide/chloride, or cetylpyridinium bromide/chloride.

It has also been found that the gas and gaseous precursor filled vesicles used in the present invention may be controlled according to size, solubility and heat stability by choosing from among the various additional or auxiliary stabilizing materials described herein. These materials can affect these parameters of the vesicles, especially vesicles formulated from lipids, not only by their physical interaction with the membranes, but also by their ability to modify the viscosity and surface tension of the surface of the gas and gaseous precursor filled vesicle. Accordingly, the gas and gaseous precursor filled vesicles used in the present invention may be favorably modified and further stabilized, for example, by the addition of one or more of a wide variety of (a) viscosity modifiers, including, for example, carbohydrates and their phosphorylated and sulfonated derivatives; polyethers, preferably with molecular weight ranges between 400 and 100,000; and di- and trihydroxy alkanes and their polymers, preferably with molecular weight ranges between 200 and 50,000; (b) emulsifying and/or solubilizing agents including, for example, acacia, cholesterol, diethanolamine, stearates, including glyceryl monostearate, lanolin alcohols, lecithin, mono- and di-glycerides, mono-ethanolamine, oleic acid, oleyl alcohol, poloxamer, for example, poloxamer 188, poloxamer 184, and poloxamer 181, poloxamine, polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, trolamine, palmitates1, and emulsifying wax; (c) suspending and/or viscosity-increasing agents, including, for example, acacia, agar, alginic acid, aluminum mono-stearate, bentonite, magma, carbomer 934P, carboxymethylcellulose, calcium and sodium and sodium 12, carrageenan, cellulose, dextran, gelatin, guar gum, locust bean gum, veegum, hydroxyethyl cellulose, hydroxypropyl methylcellulose, magnesium-aluminum-silicate, Zeolites®, methylcellulose, pectin, polyethylene oxide, povidone, propylene glycol alginate, silicon dioxide, sodium alginate, tragacanth, xanthan gum, α-d-gluconolactone, glycerol and mannitol; (d) synthetic suspending agents, such as polyethylene glycol (PEG), polyvinylpyrrolidone (PVP), polyvinylalcohol (PVA), polypropylene glycol (PPG), and polysorbate; and (e) tonicity raising agents which stabilize and add tonicity, including, for example, sorbitol, mannitol, trehalose, sucrose, propylene glycol and glycerol.

As noted above, the compositions of the present invention further comprise one or more targeting ligands. The targeting ligands which may be incorporated in the compositions of the present invention are preferably substances which are capable of targeting receptors and/or tissues in vivo. With respect to the targeting of tissue, as noted above, the targeting ligands described herein are desirably capable of targeting cells or receptors associated with vascular plaques. Thus, the methods and compositions of the present invention may be advantageously used to target plaques with diagnostic agents, thereby permitting improved detection of plaques as well as characterization of lesions in the vessels. Since these agents may accumulate preferentially in certain kinds of plaques, the present invention can be used to help detect dangerous plaques before they embolize or form thrombi on their surfaces. In this regard, the more dangerous plaques tend to have active inflammation involving the plaques. This may be manifest by the presence of inflammatory cells such as macrophages in the active plaques. Inactive plaques lacking such inflammatory cells may be less dangerous, and the methods and compositions of the present invention can be used to help differentiate between these different plaques, and to treat the more dangerous lesions.

In preferred embodiments, the targeting ligand employed in the present methods and compositions is a material which comprises an acid moiety, i.e., a moiety which may yield hydrogen ions when dissolved in water, and whose hydrogen atoms may be replaced by metals or basic radicals, or which reacts with bases to form salts and water. Such moieties include, for example, carboxylic acid moieties (—COOH), phosphoric acid moieties (—P(O)(OH)₃), sulfonic acid moieties (—SO₂OH) and the like. In preferred embodiments, the targeting ligand is in the form of a lipid containing an acid moiety, with phospholipids containing an acid moiety being more preferred. Preferred among phospholipids containing an acidic moiety, and which may be used as targeting ligands in the present compositions, are phosphatidic acids, phosphatidyl serines and phosphatidylinositols.

In connection with embodiments involving acidic phospholipids (i.e., phospholipids containing an acid moiety), the phospholipid preferably comprises at least one nonpolar aliphatic chain. Targeting ligands in the form of phospholipids which contain a single aliphatic chain are referred to herein as “monochain phospholipids.” In certain other preferred embodiments, the targeting ligands in the form of phospholipids comprise more than one, and preferably at least two or three, nonpolar aliphatic chains. Such compounds are referred to herein as “polychain phospholipids.”

In preferred embodiments, the present compositions comprise a targeting ligand in the form of a polychain phospholipid compound, with dichain phospholipids (i.e., phospholipids containing two nonpolar aliphatic chains) being preferred. More preferably, the targeting ligand comprises a diacyl phospholipid, where the number of carbons in each of the acyl groups may range, for example, from about 10 to about 20 (and all combinations and subcombinations of ranges and specific numbers of carbons therein). In preferred form, the acyl groups contain about 16 or about 17 carbons. Even more preferably, the present compositions comprise a phosphatidic acid which is dipalmitoylphosphatidic acid, a phosphatidylserine which is dipalmitoylphosphatidyl-serine (DPPS), or a phosphatidylinositol which is dipalmitoylphosphatidylinositol, in which targeting may be afforded by the acidic moiety, and stabilization may be afforded by the dipalmitoyl moieties.

In other embodiments, the targeting ligand employed in the present methods and compositions is a material which comprises a phosphorylated serine moiety. The term “phosphorylate”, as used herein, encompasses phosphate groups with various valences, including, for example, PO₂, PO₃ and PO₄ groups. Thus, in preferred form, the targeting ligands comprise a serine moiety (HOCH₂CH(NH₂)CO₂H) or a residue thereof linked to a P(O)ₓ group, where x is 2, 3 or 4. Preferably, an ester is formed between the hydroxyl group of the serine moiety and the P(O)ₓ group, although other linkages, such as a phosphoramide linkage between the amino group of the serine moiety and the P(O)ₓ group, are also encompassed within the scope of the invention and will be readily apparent to one of ordinary skill in the art, once armed with the present disclosure.

In certain preferred embodiments, the phosphorylated serine moiety may be present in a lipid compound, with phospholipids being more preferred. Embodiments in which the phosphorylated serine moiety is covalently bonded to a lipid to provide a phospholipid may be represented, for example, in connection with certain preferred embodiments, by the formula glycerol-P(O)ₓ-serine, where x is 2, 3 or 4. It is contemplated that in such embodiments, the phosphorylated serine portion targets cells or receptors associated with vascular plaque, and the lipid or glycerol portion may provide advantageous stabilizing properties to the compositions. In connection with these embodiments, the phospholipid is preferably a monochain phospholipid or a polychain phospholipid, as described above in connection with embodiments involving targeting ligands in the form of acidic phospholipids, with polychain phospholipids being preferred. More preferably, the targeting ligand comprises a diacyl phosphatidyl serine, where the number of carbons in each of the acyl groups may range, for example, from about 10 to about 20 (and all combinations and subcombinations of ranges and specific numbers of carbons therein). In preferred form, the acyl groups contain about 16 or about 17 carbons. Even more preferably, the present compositions comprise the phospholipid dipalmitoylphosphatidyl-serine (DPPS), in which targeting may be afforded by the phosphorylated serine moiety, and stabilization may be afforded by the dipalmitoyl moiety.

Also in preferred embodiments, the targeting ligand may bear a hydrophilic polymer. Thus, in embodiments in which the targeting ligand comprises, for example, a phospholipid such as a phosphatidylserine, the polymer may be linked covalently, for example, to either of the phosphate group or the serine group of the phosphorylated serine moiety. The polymer preferably has a weight average molecular weight of from about 400 to about 100,000 (and all combinations and subcombinations of molecular weight ranges and specific molecular weights therein). Suitable hydrophilic polymers include, for example, polyethylene glycol (PEG), polypropylene glycol, polyvinylalcohol, and polyvinylpyrrolidone and copolymers thereof, with PEG polymers being preferred. Preferably, the PEG polymer has a molecular weight of from about 1000 to about 20000, with molecular weights of from about 3500 to about 5000 being more preferred. The PEG or other polymer may be bound to the phosphate serine moiety through a covalent bond, such as an amide, carbamate, ether or amine linkage. In embodiments involving phosphorylated serine moieties, the resulting targeting ligand may be depicted generically by the formula PEG-P(O)ₓ-serine, where x is 2, 3 or 4.

Alternatively, the hydrophilic polymer may be linked to the lipid portion of the targeting ligand. The chemical structure of such embodiments may be depicted as PEG-glycerol-P(O)ₓ-serine, where x is 2, 3 or 4. In these embodiments, the PEG or other polymer may be covalently bonded, for example, through amide, ester, ether, thioester, thioamide or disulfide bonds. As with the lipid stabilizing materials, discussed above, where the hydrophilic polymer is PEG, a targeting ligand bearing such a polymer will be said to be “pegylated.”

Thus, in exemplary embodiments, the targeting ligand may have the formula PEG-P(O)ₓ-serine or PEG-glycerol-P(O)ₓ-serine. In accordance with these embodiments, the distal end of the PEG polymer, i.e., the end of the polymer that is not attached to the serine or glycerol moieties, may be linked or conjugated to other components of the present compositions, for example, other lipids or polymers, stabilizing materials, bioactive agents, and the like. In certain preferred embodiments, the distal end of the PEG polymer is attached to a lipid to provide a bioconjugate which may be incorporated into the vesicle walls. Such bioconjugates may be generically depicted by the formula Lipid-PEG-P(O)ₓ-serine.

Accordingly, in the case of lipid compositions, the targeting ligand, in the form of a phosphorylated serine moiety may, if desired, be bound, such as via a covalent bond, to at least one of the lipids incorporated in the compositions. In the case of vesicles which are formulated from substances other than lipids, for example, clathrates and aerogels, the targeting ligand may be bound covalently or non-covalently to one or more of the materials incorporated in the vesicle walls. If desired, the targeting ligands may also be bound covalently and/or non-covalently to other stabilizing materials, for example, biocompatible polymers, which may be present in the compositions.

In connection with targeting ligands which may be covalently bound to other components of the present compositions including, for example, lipids, polymers, proteins, vesicles, bioactive agents and the like, as well as other stabilizing materials, the targeting ligand may preferably include a functional group which may be useful, for example, in forming such covalent bonds. Examples of such functional groups include, for example, amino (—NH₂), hydroxy (—OH), carboxyl (—COOH), thiol (—SH), phosphate, phosphinate, sulfate and sulfinate.

The targeting ligands described herein, for example, targeting materials which are acidic and/or which contain a phosphorylated serine moiety, including phospholipids, such as acidic diacyl phospholipids, may be used to target any ultrasound contrast agent to vulnerable plaque. Contrast agents to be targeted may be gas filled lipid microbubbles such as developed by ImaRx Therapeutics Inc. (Tucson, Ariz.) and sold by Bristol Myers Squibb Company (New York, N.Y.) as Definity®, as described, for example, in U.S. Pat. No. 5,547,656. Other contrast agents that may be targeted to vulnerable plaque for imaging via ultrasound include polymeric shell based agents such as those described in U.S. Pat. Nos. 5,505,932 and 5,362,478; protein based shell particles such as PESDA (perfluorocarbon enhanced sonicated dextrose albumin) microbubbles, as described in U.S. Pat. Nos. 5,567,415 and 5,701,899; or Optison®, human albumin microspheres, sold by Amersham Health AS (Oslo, Norway); carbohydrate-based microparticles such as described in U.S. Pat. No. 5,928,626; and sucrose stabilized albumin microbubbles.

The targeting ligand may be incorporated in the present compositions in a variety of ways. Generally speaking, the targeting ligand may be incorporated in the present compositions by being associated covalently or non-covalently with one or more of the materials which are included in the compositions, including, for example, lipids, proteins or polymers, as well as any auxiliary stabilizing materials. For example, targeting ligands in the form of phospholipids, for example, acidic phospholipids and/or phospholipids containing phosphorylated serine moieties) may be incorporated into the ultrasound contrast agent by agitating or sonicating the acidic phospholipids along with the shell components during microbubble preparation. The concentration of the targeting phospholipids in the present contrast agents may range from about 5 to about 40 wt. % of the shell (and all combinations and subcombinations of ranges and specific percentages therein), and thus may comprise about 5%, about 10%, about 15%, about 20%, about 25%, about 30%, about 35% or about 40% of the shell material by weight percent. As described herein, the ultrasound contrast agent may be PEGylated or nonPEGylated. In certain embodiments, the lipid shelled ultrasound contrast agents may also include about 1 to about 5% cholesterol by weight, for example, to increase shell thickness.

In certain preferred embodiments, the targeting ligand is associated covalently with one or more of the aforementioned materials contained in the present compositions. As noted above, preferred compositions of the present invention comprise lipid or polymer compounds. In these compositions, the targeting ligands are preferably associated covalently with the lipid or polymer compounds.

Exemplary covalent bonds by which the targeting ligands are associated with the lipids, polymers, bioactive agents, and/or vesicles include, for example, amide (—CONH—); thioamide (—CSNH—); ether (ROR′, where R and R′ may be the same or different and are other than hydrogen); ester (—COO—); thioester (—COS—); —O—; —S—; —Sₙ—, where n is greater than 1, preferably about 2 to about 8, and more preferably about 2; carbamates; —NH—; —NR—, where R is allyl, for example, alkyl of from 1 to about 4 carbons; urethane; and substituted imidate; and combinations of two or more of these. Covalent bonds between targeting ligands and, for example, lipids, may be achieved through the use of molecules that may act as spacers to increase the conformational and topographical flexibility of the ligand. Examples of such spacers include, for example, succinic acid, 1,6-hexanedioic acid, 1,8-octanedioic acid, and the like, as well as modified amino acids, such as, for example, 6-aminohexanoic acid, 4-aminobutanoic acid, and the like. Thus, an exemplary embodiment of a bioconjugate generically depicted by the formula lipid-PEG-P(O)X-serine is distearoyldimainobutane-PEG-P(O)ₓ-serine.

The covalent linking of the targeting ligands to the materials in the present compositions, including the lipids and/or polymers, may be accomplished using synthetic organic techniques which would be readily apparent to one of ordinary skill in the art, based on the present disclosure. For example, the targeting ligands may be linked to the materials, including the lipids, via the use of well known coupling or activation agents. As known to the skilled artisan, activating agents are generally electrophilic. This electrophilicity can be employed to elicit the formation of a covalent bond. Exemplary activating agents which may be used include, for example, carbonyldiimidazole (CDI), dicyclohexylcarbodiimide (DCC), diisopropylcarbodiimide (DIC), methyl sulfonyl chloride, Castro's Reagent, and diphenyl phosphoryl chloride.

The covalent bonds may involve crosslinking and/or polymerization. Crosslinking preferably refers to the attachment of two chains of polymer molecules by bridges, composed of either an element, a group, or a compound, which join certain carbon atoms of the chains by covalent chemical bonds. For example, crosslinking may occur in polypeptides which are joined by the disulfide bonds of the cystine residue. Crosslinking may be achieved, for example, by (1) adding a chemical substance (cross-linking agent) and exposing the mixture to heat, or (2) subjecting a polymer to high energy radiation. A variety of crosslinkng agents, or “tethers”, of different lengths and/or functionalities are described, for example, in R. L. Lunbland, Techniques in Protein Modification, CRC Press, Inc., Ann Arbor, Mich., pp. 249-68 (1995), the disclosures of which are hereby incorporated herein by reference, in their entirety. Exemplary crosslinkers include, for example, 3,3′-dithiobis(succinimidylpropionate), dimethyl suberimidate, and its variations thereof, based on hydrocarbon length, and bis-N-maleimido-1,8-octane.

In certain preferred embodiments, the targeting ligands may be linked or attached to the lipids or polymers, or other stabilizing materials, via a iking group. A variety of linking groups are available and would be apparent to one skilled in the art, once armed with the present disclosure. Preferably, the linking group comprises a hydrophilic polymer. Suitable hydrophilic linker polymers include, for example, polyalkyleneoxides such as, for example, polyethylene glycol (PEG) and polypropylene glycol (PPG), polyvinylpyrrolidones, polyvinylmethylethers, polyacrylamides, such as, for example, polymethacrylamides, polydimethylacrylamides and polyhydroxypropylmethacrylamides, polyhydroxyethyl acrylates, polyhydroxypropyl methacrylates, polymethyloxazolines, polyethyloxazolines, polyhydroxyethyloxazolines, polyhyhydroxypropyloxazolines, polyvinyl alcohols, polyphosphazenes, poly(hydroxyalkylcarboxylic acids), polyoxazolidines, and polyaspartamide. The hydrophilic polymers are preferably selected from the group consisting of PEG, PPG, polyvinylalcohol and polyvinylpyrrolidone and copolymers thereof, with PEG and PPG polymers being more preferred and PEG polymers being even more prefered. Thus, in embodiments involving lipid compositions which comprise lipids bearing polymers including, for example, DPPE-PEG, the targeting ligand may be linked directly to the polymer which is attached to the lipid to provide, for example, a conjugate of DPPE-PEG-TL, where TL is a targeting ligand. Thus, using the example DPPE-PEG, such as, for example, DPPE-PEG5000, the aforementioned conjugate may be represented as DPPE-PEG5000-TL. The hydrophilic polymer used as a linking group is preferably a bifunctional polymer, for example, bifunctional PEG, such as diamino-PEG. In this case, one end of the PEG group is linked, for example, to a lipid compound, and is bound at the free end to the targeting ligand via an amide linkage. A hydrophilic polymer, for example, PEG, substituted with a terminal carboxylate group on one end and a terminal amino group on the other end, may also be used. These latter bifunctional hydrophilic polymer may be preferred since they possess various similarities to amino acids.

Standard peptide methodology may be used to link the targeting ligand to the lipid when utilizing linker groups having two unique terminal functional groups. Bifunctional hydrophilic polymers, and especially bifunctional PEGs, may be synthesized using standard organic synthetic methodologies. In addition, many of these materials are available commercially. For example, α-amino, ω-carboxy PEG is commercially available from Shearwater Polymers (Huntsville, Ala.). An advantage of using a PEG material as the linking group is that the size of the PEG can be varied such that the number of monomeric subunits of ethylene glycol may be as few as, for example, about 5, or as many as, for example, about 500 or even greater. Accordingly, the “tether” or length of the linkage may be varied, as desired. This may be important depending, for example, on the particular targeting ligand employed. For example, a targeting ligand which comprises a large protein molecule may require a short tether, such that it will simulate a membrane bound protein. A short tether would also allow for a vesicle to maintain a close proximity to the cell. This can be used advantageously in connection with vesicles which also comprise a bioactive agent, in that the concentration of bioactive agent which is delivered to the cell may be advantageously increased.

Another suitable linking group which may provide a short tether is glyceraldehyde. Glyceraldehyde may be bound, for example, to DPPE via a Schiffs base reaction. Subsequent Amadori rearrangement can provide a substantially short linking group. The β carbonyl of the Schiffs base may then react with a lysine or arginine of the targeting protein or peptide to form the targeted lipid.

More specifically, the compounds employed in the present compositions, including lipids and/or polymers, may contain various functional groups, such as, for example, hydroxy, thio and amine groups, which can react with a carboxylic acid or carboxylic acid derivative of the hydrophilic polymeric linker using suitable coupling conditions which would be apparent to one of ordinary skill in the art, once armed with the present disclosure. After the carboxylic acid group (or derivative thereof) reacts with the functional group, for example, hydroxy, thio or amine group to form an ester, thioester or amide group, any protected functional group may be deprotected utilizing procedures which would be well known to those skilled in the art. The term protecting group, as used herein, refers to any moiety which may be used to block reaction of a functional group and which may be removed, as desired, to afford the unprotected functional group. Any of a variety of protecting groups may be employed and these will vary depending, for example, as to whether the group to be protected is an amine, hydroxyl or carboxyl moiety. If the functional group is a hydroxyl group, suitable protecting groups include, for example, certain ethers, esters and carbonates. Such protecting groups are described, for example, in in Greene, TW and Wuts, PGM “Protective Groups in Organic Synthesis” John Wiley, New York, 2nd Edition (1991), the disclosures of which are hereby incorporated herein by reference, in their entirety. Exemplary protecting groups for amine groups include, for example, t-butyloxycarbonyl (Boc), benzyloxycarbonyl(Cbz), o-nitrobenzyloxycarbonyl and and trifluoroacetate (TFA).

Amine groups which may be present, for example, on a backbone of a polymer which is included in the vesicles, may be coupled to amine groups on a hydrophilic linking polymer by forming a Schiff's base, for example, by using coupling agents, such as glutaraldehyde. An example of this coupling is described by Allcock et al.,. Macromolecules Vol. 19(6), pp. 1502-1508 (1986), the disclosures of which are hereby incorporated herein by reference, in their entirety. If, for example, vesicles are formulated from polylysine, free amino groups may be exposed on the surface of the vesicles, and these free amine groups may be activated as described above. The activated amine groups can be used, in turn, to couple to a functionalized hydrophilic polymer, such as, for example, α-amino-ω-hydroxy-PEG in which the ω-hydroxy group has been protected with a carbonate group. After the reaction is completed, the carbonate group can be cleaved, thereby enabling the terminal hydroxy group to be activated for reaction to a suitable targeting ligand. In certain embodiments, the surface of a vesicle may be activated, for example, by displacing chlorine atoms in chlorine-containing phosphazene residues, such as polydichlorophosphazine. Subsequent addition of a targeting ligand and quenching of the remaining chloride groups with water or aqueous methanol will yield the coupled product.

In addition, poly(diphenoxyphosphazene) can be synthesized (Allcock et al., Macromolecules Vol. (1986) 19(6), pp. 1502-1508) and immobilized, for example, on DPPE, followed by nitration of the phenoxy moieties by the addition of a mixture of nitric acid and acetic anhydride. The subsequent nitro groups may then be activated, for example, by (1) treatment with cyanogen bromide in 0.1 M phosphate buffer (pH 11), followed by addition of a targeting ligand containing a free amino moiety to generate a coupled urea analog, (2) formation of a diazonium salt using sodium nitrite/HCl, followed by addition of the targeting ligand to form a coupled ligand, and/or (3) the use of a dialdehyde, for example, glutaraldehyde as described above, to form a Schiff's base. After linking the DPPE to the hydrophilic polymer and the targeting ligand, the vesicles may be formulated utilizing the procedures described herein.

Aldehyde groups on polymers can be coupled with amines as described above by forming a Schiff's base. An example of this coupling procedure is described in Allcock and Austin Macromolecules vol 14. p1616 (1981), the disclosures of which are hereby incorporated herein by reference, in their entirety.

In the above procedures, the polymer or terminus of the lipid, for example, phosphatidylglycerol or phosphatidylethanolamine, is preferably activated and coupled to the hydrophilic polymeric linker, the terminus of which has been blocked in a suitable manner. As an example of this strategy, α-amino, ω-carboxy PEG4000 having a t-Boc protected terminal amino group and a free carboxylate end, may be activated with 1,1′-carbonyldiimidazole in the presence of hydroxybenzotriazole in N-methylpyrollidone. After the addition of phosphatidylethanolamine, the t-Boc group may be removed by using trifluoroacetic acid (TFA), leaving the free amine. The amine may then be reacted with a targeting ligand by similar activation of the ligand, to provide the lipid-linker-targeting ligand conjugate. Other strategies, in addition to those exemplified above, may be utilized to prepare the lipid-linker-targeting ligand conjugates. Generally speaking, these methods employ synthetic strategies which are generally known to those skilled in the art of synthetic organic chemistry.

Additional linkers would include other derivatives of lipids useful for coupling to a bifunctional spacer. For example, phosphatidylethanolamine (PE) may be coupled to a bifunctional agent. For example N-succinimidyl 4-(p-maleimido-phenyl)butyrate (SMPB) and N-succinimidyl 3-(2-pyridyldithiol) propionate (SPDP), N-succinimidyl trans-4-(N-maleimidyhnethyl)cyclohexane-1-carboxylate (SMCC), and N-succinimidyl 3-maleimidylbenzoate (SMB) may be used among others, to produce, for example the functionalized lipids MPB-PE and PDP-PE.

The free end of the hydrophilic spacer, such as polyethylene glycol ethylamine, which contains a reactive group, such as an amine or hydroxyl group, could be used to bind a cofactor or other targeting ligand. For example, polyethylene glycol ethylamine may be reacted with N-succinimidylbiotin or p-nitrophenylbiotin to introduce onto the spacer a useful coupling group. For example, biotin may be coupled to the spacer and this will readily bind non-covalently proteins. As an example, MPB-PEG-DPPE may be synthesized as follows. DPPE-PEG with a free amino group at the terminus of the PEG will be provided as described previously. Synthesis of the SMPB:PEG-DPPE may then be carried out with 1 equivalent of triethylamine in chloroform at a molar ratio of 1:5 SMPB:DPPE-PEG. After 3 hours, the reaction mixture will be evaporated to dryness under argon. Excess unreacted SMPB and major by products will be removed by preparative thin layer chromatography (TLC, silica gel developed with 50% acetone in chloroform). The upper portion of the lipid band can be extracted from the silica with about 20-30% methanol in chloroform (V:V) resulting in the isolation of pure intact MPB-Peg-DPPE. Streptavidin may then be coupled to proteins so that the proteins in turn may then be coupled to the MPB-PEG-DPPE. Briefly SPDP would be incubated with streptavidin at room temperature for 30 minutes and chromatography employed to remove unreacted SPDP. Dithiothreitol (DTT) was added to the reaction mixture and 10 minutes later 2-thiopyridone at a concentration of 343 nM. The remainder of the reaction mixture is reduced with DTT (25 mM for 10 min.). The thiolated product is isolated by gel exclusion. The resulting streptavidin labeled proteins may then be used to bind to the biotinylated spacers affixed to the lipid moieties.

Additional methods which may be employed for covalently linking targeting ligands to the lipids or polymers, vesicles, bioactive agents or other stabilizing materials, are described, for example, in Unger, et al., U.S. Pat. No. 6,090,800 and Unger U.S. Pat. No. 6,028,066, the disclosures of which are incorporated herein by reference, in their entireties.

In preferred embodiments of the present invention, the targeted compounds, namely, targeted lipids and polymers, may be incorporated in compositions which are used to form targeted vesicles, including, for example, targeted micelles, targeted liposomes, targeted protein microspheres and/or targeted polymer coated microspheres. The targeting ligand which is attached to the compounds from which the vesicles are prepared may be directed, for example, outwardly from the surface of the vesicle. Thus, there is provided a targeted vesicle which can be used to target receptors and tissues.

The concentration of targeting ligand employed in the present compositions may vary depending, for example, on the particular targeting agent employed, the receptor or tissue being targeted, the other components of the compositions, whether the targeting ligand is associated covalently and/or non-covalently with other components of the compositions, for example, lipids, polymers or vesicles and the like. Typically, the concentration of targeting ligand in the present compositions may be initiated at lower levels and increased until the desired contrast enhancement effect is achieved. Targeted hydrophobic compounds, for example, conjugates in which the targeting ligand is linked covalently to, for example, a lipid, may be employed in the compositions in a concentration which ranges from about 0.05 wt % to about 40 wt % (and all combinations and subcombinations of ranges therein and specific percentages therein), based on the weight of other stabilizing materials employed in the composition. Preferably, the targeting ligand may be employed in the compositions in a concentration from about 0.5 wt % to about 30 wt %, with concentrations of from about 1 wt % to about 20 wt % being more preferred. Even more preferably, the targeting ligand may be employed in the present compositions in a concentration of from about 1 wt % to about 2 wt %, with a concentration of about 1.25 wt % being especially preferred. In other embodiments, concentrations of about 5 wt % may be especially preferred.

As would be apparent to the skilled artisan, once armed with the teachings of the present invention, the concentration of targeted hydrophobic compounds and/or unbound targeting ligand that may be employed in the compositions of the present invention may also be expressed in mole %. In this connection, targeted hydrophobic compounds and/or unbound or free targeting ligand may be employed in the compositions of the present invention in a concentration which ranges from 0.05 mole % to about 30 mole % (and all combinations and subcombinations of ranges and specific percentages therein), based on the number of moles of unbound stabilizing materials employed in the composition. Preferably, the conjugate or free targeting ligand may be employed in the compositions in a concentration from about 0.5 mole % to about 20 mole %, with concentrations of from about 1 mole % to about 5 mole % being more preferred. Even more preferably, the conjugate or free targeting ligand may be employed in the present compositions in a concentration of from about 1 mole % to about 2 mole %, with a concentration of about 1.8 mole % being especially preferred.

As noted above, the present lipid and/or vesicle compositions are desirably formulated in an aqueous environment. This can induce the lipid, because of its hydrophobic/hydrophilic nature, to form vesicles, which may be the most stable configuration which can be achieved in such an environment. In the case of phospholipids, the polar head groups may orient themselves towards the surface of the vesicles, while the nonpolar hydrophobic portion is oriented towards the interior of the vesicle. Thus, in the case of vesicles which comprise phosphorylated serine groups, the phosphorylated serine moiety may be advantageously exposed on the surface of the veiscle, thereby permitting interaction with the desired target, for example, plaque or macrophages associated therewith. The diluents which can be employed to create such an aqueous environment include, for example, water, including deionized water or water containing one or more dissolved solutes, such as salts, which preferably do not interfere with the formation and/or stability of the vesicles or their use as diagnostic agents, such as ultrasound contrast agents, MRI contrast agents or CT contrast agents; and normal saline and physiological saline.

As noted above, the present compositions may further optionally comprise an oil. For purposes of the present disclosure, the terms “oil”, “oils”, and variations thereof as used throughout the application, will be understood to include waxes and fats. Preferred oils, waxes and fats are those having melting points under 100° C. Especially preferred are synthetic oils with melting points between −20° C. and 66° C., more preferably those melting less than 60° C., and most preferably those melting less than 42° C. In this aspect, oils and waxes are generally used to dissolve bioactive agents, but may also be used to suspend crystals of dried bioactive agents, e.g., etoposide or bleomycin. Waxes melting at temperatures above 60° C. may also be used, but generally lower melting point waxes are preferred. Many natural oils known in the literature may be useful in the present invention. The melting points of some conventional oils are difficult to determine due to a multicomponent nature which decompose upon state change.

Preferred among the oils useful in the methods and compositions of the present invention are low viscosity oils. The term “low viscosity”, as used herein, refers to materials which may have a viscosity at ambient room temperature ranging from about 1 centipoise to about 4,000 centipoise, and all combinations and subcombinations of ranges and specific viscosities therein. Preferably, the low viscosity oils have a viscosity at ambient room temperature of from about 1 centipoise to about 2,000 centipoise, with viscosities of about 1 to about 1,000 centipoise being preferred, and viscosities of about 1 to about 500 centipoise or less being more preferred. Low viscosity oils may be desirably capable of maintaining the acoustic properties of the contrast agents, particularly in connection with contrast agents based on lipids, for example, lipid based microbubbles. Particularly preferred low viscosity oils include, for example, triacetin, diacetin, and low viscosity mineral oils.

Other commercially-available synthetic oils and surfactants are also suitable to substitute for the oils listed above. Among such oils are those listed in Owen et al., U.S. Pat. No. 5,633,226, the disclosure of which is hereby incorporated by reference herein, and include Captex 200 (a composition described in 5,633,226), Whitepsol H-15 and MYVACET 9-45K. Surfactants which can optionally be included from the same reference include capmul MCM, Myverol 18-92, Cremophor EL, Centrophase 31, derivatives of polyoxyethylene, and those disclosed in Brown, U.S. Pat. No. 5,573,781 of Brown, the disclosure of which are hereby incorporated herein by reference in its entirety.

Examples of oils, waxes and fasts suitable for the microspheres of the invention include, but are not limited, to those listed in the following table:

TABLE 1
MELTING POINTS OF WAXES, FATS AND OILS (° C.)

| | Melting | |
|---|---|---|
| | Point | Natural Origin |
| Oils/Waxes/Fats | | |
| Jojoba | 11 | |
| Cay-cay | 30 | |
| Woolwax (anhydrous lanolin) | 39.5 | |
| Ucuhuba | 42.5 | |
| Spermaceti | 44 | |
| Hydrogenated Cocoa Oil | 44 | |
| Parrafin | 45-68 | |
| Orange skin | 46 | |
| Bayberry | 47 | |
| Cetyl alchol | 49 | |
| Japanwax | 49-52 | |
| Sorbitol distearate | 50 | |
| Lanette Wax | 50 | |
| Spermafol 52 | 51 | |
| Cetyl palmitate | 52 | |
| Insect wax (Ceroplastes) | 55 | |
| Diglycol stearate | 56.5 | |
| Indian Arjun | 59 | |
| Pliowax | 55.5 | |
| Ponderosa bark | 58 | |
| Chinese tallow tree | 57 | |
| Carbowax stearate | 57 | |
| Cetyl acetamide | 59 | |
| Jasmine floral | 60 | |
| Beeswax | 62 | |
| Saturated Fatty Acids | | |
| Formic | 8.4 | |
| Acetic | 16.6 | |
| Propionic | −22 | |
| Butyric | −8 | Milk fat |
| Valeric | −34.5 | |
| Caproic | −3.4 | Coconut Oil, 0.5% |
| Enanthic | −7.5 | |
| Caprylic | 16.7 | Coconut Oil, 9% |
| Pelargonic | 12.3 | |
| Capric | 31.6 | Coconut Oil, Elm Seed Oil |
| Hendecanoic | 28.5 | |
| Lauric | 44.2 | Coconut Oil, Palm Kernal Oil |
| Tridecanoic | 41.5 | |
| Myristic | 54.4 | Nutmeg Fat |
| Pentadecanoic | 52.3 | |
| Palmitic | 62.9 | Palm Oil, Cottonseed Oil |
| Unsaturated Fatty Acids | | |
| Linderic | 5.3 | Seed Fat |
| Tsuzuic | 18.5 | Seed Fat |
| Palmitoleic | 0.5 | Soybean Oil, Sea Algaes |
| Petroselinic | 30 | Parsley seed oil |
| Oleic | 16.3 | Widely distributed |
| Elaidic | 43.7 | Partially hydrogenated fats |
| Erucic | 33.5 | Mustard Seed Oil |
| Brassidic | 60 | trans Isomer of Erucic |
| Linoleic | −5 | very widely distributed |
| Linolenic | −11 | Linseed oils |
| Santalbic | 42 | seed fat |
| α-Eleostearic | 49 | Tung oil |
| Punicic | 44 | Pomegranite seed oil |
| Synthetic Fats | | |
| Triolein | −4.5 | |
| Trimyristin | 56 | |
| Triacetin | −78 | |
| Tripalmitin | 66 | |
| Tristearin | 55 | |
| Tributyrin | −75 | |
| Glyceryl Monooctanoate | 29 | |
| Glyceryl Monosterate | 57 | |
| Natural Fats | | |
| Beef tallow | 43 | |
| Mutton tallow | 47 | |
| Lard | 41.5 | |
| Butter | 31 | |
| Cacao Butter | 24.5 | |
| Laurel Oil | 33 | |
| Palm Oil | 30 | |
| Cocoa Nut Oil | 24 | |
| Nutmeg Butter | 43.5 | |
| Soybean Oil | −13 | |
| | (average) | |
| Rapeseed Oil | −6 | |
| | (average) | |
| Corn Oil | −14 | |
| | (average) | |
| Castor Oil | −14 | |
| | (average) | |
| Japanese Anise Oil | −12.5 | |
| | (average) | |
| Oil of Eucalyptus | −15.5 | |
| Mustard Seed Oil | −12 | |
| | (average) | |
| Rose Oil | 20 | |
| Almond Oil | −20 | |

Fluorinated triglyceride oils may be prepared by reacting a reactive fluorinated species, such as for example, a fluorine gas, with unsaturated triglyceride oils to produce the desired fluorinated triglyceride.

A wide variety of diagnostic agents may be targeted to plaques using the methods and compositions of the present invention. Exemplary of such diagnostic agents include, for example, gas and/or gaseous precursor filled vesicles for ultrasound imaging, radionuclides for nuclear medicine, paramagnetic and superparamagnetic materials for magnetic resonance imaging, radiodense materials for X-ray imaging, optically active materials for optical imaging and combinations agents for Hall effect imaging and optoacoustic imaging.

It is contemplated that the compositions of the present invention are particularly useful in connection with ultrasound, including diagnostic and therapeutic ultrasound. The use of the present compositions in ultrasound is described throughout the present disclosure.

As noted above, the present compositions may also be employed in connection with computed tomography (CT) imaging. CT suffers from various drawbacks, and is generally less effective as compared to the diagnostic techniques discussed above. Nevertheless, if a high enough concentration of the present contrast media, and especially gas filled vesicle compositions, is delivered to the region of interest, for example, a blood clot, the clot can be detected on the CT images by virtue of a decrease in the overall density of the clot. In general, a concentration of about 1/10 of 1% of gas filled vesicles or higher (on a volume basis), may be needed to delivered to the region of interest, including the aforementioned blood clot, to be detected by CT.

Exemplary paramagnetic and superparamagnetic contrast agents suitable for use in the present compositions include, for example, stable free radicals, such as, for example, stable nitroxides, as well as compounds comprising transition, lanthamide and actinide elements, which may, if desired, be in the form of a salt or may be covalently or non-covalently bound to complexing agents, including lipophilic derivatives thereof, or to proteinaceous macromolecules.

Preferable transition, lanthamide and actinide elements include, for example, Gd(M), Mn(II), Cu(II), Cr(II), Fe(II), Fe(III), Co(II), Er(II), Ni(II), Eu(III) and Dy(III). More preferably, the elements may be Gd(III), Mn(II), Cu(II), Fe(II), Fe(III), Eu(III) and Dy(III), especially Mn(II) and Gd(III). Additional paragmagnetic and superparamagnetic materials are set forth in U.S. Pat. No. 5,312,617, the disclosure of which is hereby incorporated by reference herein, in its entirety.

The foregoing elements may, if desired, be in the form of a salt, including inorganic salts, such as a manganese salt, for example, manganese chloride, manganese carbonate, manganese acetate, and organic salts, such as manganese gluconate and manganese hydroxylapatite. Other exemplary salts include salts of iron, for example, iron sulfides and ferric salts such as ferric chloride.

These elements may also, if desired, be bound, for example, through covalent or noncovalent association, to complexing agents, including lipophilic derivatives thereof, or to proteinaceous macromolecules. Preferable complexing agents include, for example, diethylenetriaminepentaacetic acid (DTPA), ethylene-diaminetetraacetic acid (EDTA), 1,4,7,10-tetraazacyclododecane-N,N′,N′,N′″-tetraacetic acid (DOTA), 1,4,7,10-tetrazacyclododecane-N,N′,N″-triacetic acid (DOTA), 3,6,9-triaza-12-oxa-3,6,9-tricarboxymethylene-10-carboxy-13-phenyl-tridecanoic acid (B-19036), hydroxybenzylethylenediamine diacetic acid (HBED), N,N-bis(pyridoxyl-5-phosphate)ethylene diamine, N,N′-diacetate (DPDP), 1,4,7-triazacyclononane-N,N′,N″-triacetic acid (NOTA), 1,4,8,11-tetraazacyclotetradecane-N,N′,″,N′″-etraaeetic acid (TETA), kryptands (macrocyclic complexes), and desferrioxamine. More preferably, the complexing agents are EDTA, DTPA, DOTA, DO3A and kryptands, most preferably DTPA. Preferable lipophilic complexes include alkylated derivatives of the complexing agents EDTA, DOTA, for example, N,N-bis-(carboxydecylamidomethyl-N-2,3-dihydroxypropyl)-ethylenediamine-N,N-diacetate (EDTA-DDP); N,N-bis-(carboxy-octadecylamido-methyl-N-2,3-dihydroxypropyl)ethylenediamine-N,N′-diacetate (EDTA-ODP); N,-Bis(carboxy-laurylamidomethyl-N-2,3-dihydroxypropyl)ethylenediamine-N,N-diacetate (EDTA-LDP); and the like, including those described in U.S. Pat. No. 5,312,617, the disclosures of which are hereby incorporated herein by reference, in their entirety. Preferable proteinaceous macromolecules include, for example, albumin, collagen, polyarginine, polylysine, polyhistidine, γ-globulin and β-globulin, with albumin, polyarginine, polylysine, and polyhistidine being more preferred.

Suitable complexes therefore include Mn(II)-DTPA, Mn(II)-EDTA, Mn(II)-DOTA, Mn(II)-DO3A, Mn(II)-kryptands, Gd(III)-DTPA, Gd(III)-DOTA, Gd(III)-DO3A, Gd(III)-kryptands, Cr(III)-EDTA, Cu(II)-EDTA, or iron-desferrioxamine, especially Mn(II)-DTPA or Gd(III)-DTPA.

Nitroxides are paramagnetic contrast agents which increase both T1 and T2 relaxation rates on MRI by virtue of the presence of an unpaired electron in the nitroxide molecule. As known to one of ordinary skill in the art, the paramagnetic effectiveness of a given compound as an MRI contrast agent may be related, at least in part, to the number of unpaired electrons in the paramagnetic nucleus or molecule, and specifically, to the square of the number of unpaired electrons. For example, gadolinium has seven unpaired electrons whereas a nitroxide molecule has one unpaired electron. Thus, gadolinium is generally a much stronger MRI contrast agent than a nitroxide. However, effective correlation time, another important parameter for assessing the effectiveness of contrast agents, confers potential increased relaxivity to the nitroxides. When the tumbling rate is slowed, for example, by attaching the paramagnetic contrast agent to a large molecule, it will tumble more slowly and thereby more effectively transfer energy to hasten relaxation of the water protons. In gadolinium, however, the electron spin relaxation time is rapid and will limit the extent to which slow rotational correlation times can increase relaxivity. For nitroxides, however, the electron spin correlation times are more favorable and tremendous increases in relaxivity may be attained by slowing the rotational correlation time of these molecules. The gas filled vesicles of the present invention are ideal for attaining the goals of slowed rotational correlation times and resultant improvement in relaxivity. Although not intending to be bound by any particular theory of operation, it is contemplated that since the nitroxides may be designed to coat the perimeters of the vesicles, for example, by making alkyl derivatives thereof, the resulting correlation times can be optimized. Moreover, the resulting contrast medium of the present invention may be viewed as a magnetic sphere, a geometric configuration which maximizes relaxivity.

If desired, the nitroxides may be alkylated or otherwise derivatized, such as the nitroxides 2,2,5,5-tetramethyl-1-pyrrolidinyloxy, free radical, and 2,2,6,6-tetramethyl-1-piperidinyloxy, free radical (TMPO).

Exemplary superparamagnetic contrast agents suitable for use in the compositions of the present invention include metal oxides and sulfides which experience a magnetic domain, ferro- or ferrimagnetic compounds, such as pure iron, magnetic iron oxide, such as magnetite, γ-Fe₂O₃, Fe₃O₄, manganese ferrite, cobalt ferrite and nickel ferrite. Paramagnetic gases can also be employed in the present compositions, such as oxygen 17 gas (¹⁷O₂). In addition, hyperpolarized xenon, neon, or helium gas may also be employed. MR whole body imaging may then be employed to rapidly screen the body, for example, for thrombosis, and ultrasound may be applied, if desired, to aid in thrombolysis.

The contrast agents, such as the paramagnetic and superparamagnetic contrast agents described above, may be employed as a component within the lipid and/or vesicle compositions. In the case of vesicle compositions, the aforementioned contrast agents may be entrapped within the internal void thereof, administered as a solution with the vesicles, incorporated with any additional stabilizing materials, or coated onto the surface or membrane of the vesicle.

If desired, the paramagnetic or superparamagnetic agents may be delivered as alkylated or other derivatives incorporated into the compositions, especially the lipidic walls of the vesicles. In particular, the nitroxides 2,2,5,5-tetramethyl-1-pyrrolidinyloxy, free radical and 2,2,6,6-tetramethyl-1-piperidinyloxy, free radical, can form adducts with long chain fatty acids at the positions of the ring which are not occupied by the methyl groups via a variety of linkages, including, for example, an acetyloxy linkage. Such adducts are very amenable to incorporation into the lipid and/or vesicle compositions of the present invention.

Mixtures of any one or more of the paramagnetic agents and/or superparamagnetic agents in the present compositions may be used. The paramagnetic and superparamagnetic agents may also be coadministered separately, if desired.

The lipid and/or vesicle compositions of the present invention, and especially the vesicle compositions, may serve not only as effective carriers of the superparamagnetic agents described above, but also may improve the effect of the susceptibility contrast agents. Superparamagnetic contrast agents include metal oxides, particularly iron oxides but including manganese oxides, and as iron oxides, containing varying amounts of manganese, cobalt and nickel which experience a magnetic domain. These agents are nano or microparticles and have very high bulk susceptibilities and transverse relaxation rates. The larger particles, for example, particles having diameters of about 100 nm, have much higher R2 relaxivities as compared to R1 relaxivities. The smaller particles, for example, particles having diameters of about 10 to about 15 nm, have somewhat lower R2 relaxivities, but much more balanced R1 and R2 values. Much smaller particles, for example, monocrystalline iron oxide particles having diameters of about 3 to about 5 nm, have lower R2 relaxivities, but probably the most balanced R1 and R2 relaxation rates. Ferritin can also be formulated to encapsulate a core of very high relaxation rate superparamagnetic iron. It has been discovered that the lipid and/or vesicle compositions, especially vesicle compositions, including gas filled vesicles, can increase the efficacy and safety of these conventional iron oxide based MRI contrast agents.

The iron oxides may simply be incorporated into the lipid and/or vesicle compositions. Preferably, in the case of vesicles formulated from lipids, the iron oxides may be incorporated into the walls of the vesicles, for example, by being adsorbed onto the surfaces of the vesicles, or entrapped within the interior of the vesicles as described in U.S. Pat. No. 5,088,499, the disclosures of which are hereby incorporated herein by reference in their entirety.

Without being bound to any particular theory or theories of operation, it is believed that the vesicles of the present invention increase the efficacy of the superparamagnetic contrast agents by several mechanisms. First, it is believed that the vesicles function to increase the apparent magnetic concentration of the iron oxide particles. Also, it is believed that the vesicles increase the apparent rotational correlation time of the MRI contrast agents, including paramagnetic and superparamagnetic agents, so that relaxation rates are increased. In addition, the vesicles appear to increase the apparent magnetic domain of the contrast medium according to the manner described hereinafter.

Certain of the vesicles of the present invention, and especially vesicles formulated from lipids, may be visualized as flexible spherical domains of differing susceptibility from the suspending medium, including, for example, the aqueous suspension of the contrast medium or blood or other body fluids, for example, in the case of intravascular injection or injection into other body locations. In the case of ferrites or iron oxide particles, it should be noted that the contrast provided by these agents is dependent on particle size. This phenomenon is very common and is often referred to as the “secular” relaxation of the water molecules. Described in more physical terms, this relaxation mechanism is dependent upon the effective size of the molecular complex in which a paramagnetic atom, or paramagnetic molecule, or molecules, may reside. One physical explanation may be described in the following Solomon-Bloembergen equations which define the paramagnetic contributions as a function of the T₁ and T₂relaxation times of a spin ½ nucleus with gyromagnetic ratio g perturbed by a paramagnetic ion:
1/T₁M=(2/15)S(S+1)β²g²β²/r⁶[3τ_{c}/(1+ω_{I}²τ_{c}²)+7τ_{c}/(1+ωₛ²τ_{c}²)]+(⅔)S(S+1)A²/h² [τₑ/(1ωₛ2τₑ²)]
and
1/T₂M=(1/15)S(S+1)γ²g²β²/r⁶[4τ_{c}+3τc/(1ω_{I}²τ_{c}²)+13τ_{c}/(1+wₛ²τ_{c}²)]+(⅓)S(S+1)A²/h²[τₑ/(1+ωₛ2τₑ²)]
where:
S is the electron spin quantum number;
g is the electronic g factor;
β is the Bohr magneton;
ωI and ωₛ (657 w_{I}) is the Larmor angular precession frequencies for the nuclear spins and electron spins;
r is the ion-nucleus distance;
A is the hyperfine coupling constant;
τ_{c} and τₑ are the correlation times for the dipolar and scalar interactions, respectively; and
h is Planck's constant.
   See, e.g., Solomon, I. Phys. Rev. Vol. 99, p. 559 (1955) and Bloembergen, N. J. Chem. Phys. Vol. 27, pp. 572, 595 (1957).

A few large particles may have a much greater effect than a larger number of much smaller particles, primarily due to a larger correlation time. If one were to make the iron oxide particles very large however, increased toxicity may result, and the lungs may be embolized or the complement cascade system may be activated. Furthermore, it is believed that the total size of the particle is not as important as the diameter of the particle at its edge or outer surface. The domain of magnetization or susceptibility effect falls off exponentially from the surface of the particle. Generally speaking, in the case of dipolar (through space) relaxation mechanisms, this exponential fall off exhibits an r⁶ dependence for a paramagnetic dipole-dipole interaction. Interpreted literally, a water molecule that is 4 angstroms away from a paramagnetic surface will be influenced 64 times less than a water molecule that is 2 angstroms away from the same paramagnetic surface. The ideal situation in terms of maximizing the contrast effect would be to make the iron oxide particles hollow, flexible and as large as possible. It has not been possible to achieve this heretofore and it is believed that the benefits have been unrecognized heretofore also. By coating the inner or outer surfaces of the vesicles with the contrast agents, even though the individual contrast agents, for example, iron oxide nanoparticles or paramagnetic ions, are relatively small structures, the effectiveness of the contrast agents may be greatly enhanced. In so doing, the contrast agents may function as an effectively much larger sphere wherein the effective domain of magnetization is determined by the diameter of the vesicle and is maximal at the surface of the vesicle. These agents afford the advantage of flexibility, namely, compliance. While rigid vesicles might lodge in the lungs or other organs and cause toxic reactions, these flexible vesicles slide through the capillaries much more easily.

In contrast to the flexible vesicles described above, it may be desirable, in certain circumstances, to formulate vesicles from substantially impermeable polymeric materials including, for example, polymethyl methacrylate. This would generally result in the formation of vesicles which may be substantially impermeable and relatively inelastic and brittle. In embodiments involving diagnostic imaging, for example, ultrasound, contrast media which comprise such brittle vesicles would generally not provide the desirable reflectivity that the flexible vesicles may provide. However, by increasing the power output on ultrasound, the brittle microspheres can be made to rupture, thereby causing acoustic emissions which can be detected by an ultrasound transducer.

Nuclear Medicine Imaging (NMI) may also be used in connection with the diagnostic and therapeutic method aspects of the present invention. For example, NMI may be used to detect radioactive gases, such as Xe¹³³, which may be incorporated in the present compositions in addition to, or instead of, the gases discussed above. Such radioactive gases may be entrapped within vesicles for use in detecting, for example, thrombosis. Preferably, bifunctional chelate derivatives are incorporated in the walls of vesicles, and the resulting vesicles may be employed in both NMI and ultrasound. In this case, high energy, high quality nuclear medicine imaging isotopes, such as technitium, indium, iodine, gallium and other radioactive elements can be incorporated in the walls of vesicles. Whole body gamma scanning cameras can then be employed to rapidly localize regions of vesicle uptake in vivo. If desired, ultrasound may also be used to confirm the presence, for example, of a plaque within the blood vessels, since ultrasound generally provides improved resolution as compared to nuclear medicine techniques. NMI may also be used to screen the entire body of the patient to detect areas of vascular thrombosis, and ultrasound can be applied to these areas locally to promote rupture of the vesicles and thereby treat the plaque. In embodiments involving the aforementioned isotopes, the compositions may further comprise chelates such as EDTA, DTPA, DOTA and other macrocycles. Additional suitable chelates are disclosed in U.S. Pat. No. 5,458,127, the disclosure of which is hereby incorporated herein by reference, in its entirety. For PET scanning, the present compositions may be used to deliver PET isotopes of fluorine and oxygen, rubidium and other positron emitting isotopes. For therapy with radioactivity, the present compositions may be used to deliver therapeutic isotopes such as yttrium and strontium.

For X-ray imaging, the present compositions may be used to deliver agents with a difference in radiodensity between the plaque being targeted and the agent being so employed. Suitable agents may comprise a low density material, including the gases described above, but is more preferably a metal ion with high radiodensity, e.g., iodinated agents such as iothalamate, ioxaglate and the like, or other high density materials such as bismuth, lead, strontium and tungsten. For optical imaging, the compositions may be employed with time of flight imaging (e.g. using reflected light), transmitted light imaging, optical coherence tomography as well as other optical imaging techniques. The materials carried by the targeting ligands to plaque will change the acoustic reflectivity or absorbtivity of the plaque. Such materials may comprise gaseous bodies, such as argon or neon, or metal ions but preferably will comprise fluorescent materials, including porphyrin derivatives, may also be used. Additional photosensitive agents which may be employed in the present compositions are described in U.S. Pat. No. 6,123,923, the disclosure of which is hereby incorporated herein by reference, in its entirety.

Elastography is an imaging technique which generally employs much lower frequency sound, for example, about 60 KHz, as compared to ultrasound which can involve over frequencies of over 1 MHz. In elastography, the sound energy is generally applied to the tissue and the elasticity of the tissue may then be determined. In connection with preferred embodiments of the invention, which involve highly elastic vesicles, the deposition of such vesicles onto, for example, a vascular plaque, increases the local elasticity of the tissue and/or the space surrounding the plaque. This increased elasticity may then be detected with elastography. If desired, elastography can be used in conjunction with other imaging techniques, such as MRI and ultrasound.

In a combination type imaging, the present compositions may be used to deliver one or more agents for interaction with multiple energy sources. In Hall effect imaging, a tissue may be vibrated within a magnetic field. Such vibration may be accomplished with ultrasound imaging, e.g. by insonating a tissue within a magnetic field. By incorporating a material that changes the magnetic properties, acoustic properties or electrical properties of the material, then in a Hall effect imaging regime the targeted contrast agents may amplify the signal from the plaque. Similarly, although using light and sound, optoacoustic imaging, can be used to detect carriers bound to the plaque when such materials are optically and/or acoustically active. Methods and apparatus which may be suitable for carrying out combination type imaging are disclosed, for example, in U.S. Pat. No. 5,558,092, the disclosure of which is hereby incorporated herein by reference, in its entirety.

The targeted compositions of the present invention may also be used to deliver electrically active materials for electrical impedance imaging. Among these agents are physiologically acceptable salts of 3-acetylamino-2,4,6-triiodobenzoic acid, 3,5-diacetamido-2,4,6-triiodobenzoic acid, 2,4,6-triiodo-3,5-dipropionamido-benzoic acid, 3-acetylamino-5-((acetylamino)methyl)-2,4,6-triiodobenzoic acid, 3-acetylamino-5-(acetylmethylamino)-2,4,6-triiodobenzoic acid, 5-acetamido-2,4,6-triiodo-N-((methylcarbamoyl)methyl)-isophthalamic acid, 5-(2-methoxyacetamido)-2,4,6-triiodo-N-[2-hydroxy-1-(methylcarbamoyl)-ethyl]-isophthalamicacid, 5-acetamido-2,4,6-triiodo-N-methylisophthalamic acid, 5-acetamido-2,4,6-triiodo-N-(2-hydroxyethyl)-isophthalamic acid, 2-[[2,4,6-triiodo-3-[(1-oxobutyl)-amino]phenyl]methyl]butanoic acid, beta-(3-amino-2,4,6-triiodophenyl)-alpha-ethyl-propanoic acid, 3-ethyl-3-hydroxy-2,4,6-triiodophenylpropanoic acid, 3-[[(dimethylamino)-methyl]amino]-2,4,6-triiodophenyl-propanoic acid (see Chem. Ber. 93:2347 (1960)), alpha-ethyl-(2,4,6-triiodo-3-(2-oxo-1-pyrrolidinyl)-phenyl)-propanoic acid, 2-[2-[3-(acetylamino)-2,4,6-triiodophenoxy]ethoxymethyl]butanoic acid, N-(3-amino-2,4,6-triiodobenzoyl)-N-phenyl-beta-aminopropanoic acid, 3-acetyl-(3-amino-2,4,6-triiodophenyl)amino]-2-methylpropanoic acid, 5-[(3-amino-2,4,6-triiodophenyl)methylamino]-5-oxypentanoic acid, 4-[ethyl-[2,4,6-triiodo-3-(methylamino)phenyl]amino]-4-oxo-butanoic acid, 3,3′-oxybis[2,1-ethanediyloxy-(1-oxo-2,1-ethanediyl)imino]bis-2,4,6-triiodobenzoic acid, 4,7,10,13-tetraoxahexadecane-1,16-dioyl-bis(3-carboxy-2,4,6-triiodoanilide), 5,5′-(azelaoyldiimino)-bis[2,4,6-triiodo-3-(acetylamino)methyl-benzoic acid, 5,5′-(apidoldiimino)bis(2,4,6-triiodo-N-methyl-isophthalamic acid), 5,5′-(sebacoyl-diimino)-bis(2,4,6-triiodo-N-methylisophthalamic acid), 5,5-[N,N-diacetyl-(4,9-dioxy-2, 11-dihydroxy-1,12-dodecanediyl)dii ino]bis(2,4,6-triiodo-N-methylisophthalamic acid), 5,5′5″-(nitrilo-triacetyltrii nino)tris(2,4,6-triiodo-N-methyl-isophthalamic acid), 4-hydroxy-3,5-diiodo-alpha-phenylbenzenepropanoic acid, 3,5-diiodo4-oxo-1 (4H)-pyridine aceticacid, 1,4-dihydro-3,5-diiodo-1-methyl-4-oxo-2,6-pyridinedicarboxylicacid, and 5-iodo-2-oxo-[(2H)-pyridine acetic acid, and N-(2-hydroxyethyl)-2,4,6-triiodo-5-[2-[2,4,6-triiodo-3-(N-methylacetamido)-5-(methylcarbomoyl)benzamino]acetamido]-isophthalamic acid.

Other EII contrast agents include ionic compounds (such as for example GdDTPA and GdDOTA) which have been proposed for use as MRI contrast agents, especially the salts of paramagnetic metal complexes (preferably chelate complexes) with physiologically compatible counterions, as well as similar complexes in which the complexed metal ion is diamagnetic (as paramagnetism is not a property required for the EII contrast agent to function as such). Preferred complexed paramagnetic metal ions include ions of Gd, Dy, Eu, Ho, Fe, Cr and Mn and preferred non paramagnetic complexed ions include ions of Zn, Bi and Ca

The complexing agent will preferably be a chelating agent such as a linear, branched or cyclic polyamine or a derivative thereof, e.g. a polyaminocarboxylic acid or a polyaminopolyphosphonic acid or a derivative of such an acid, e.g. an amide or ester thereof. Particular mention in this regard may be made of DTPA, DTPA-bisamides (e.g. DTPA-bismethylamide and DTPA-bismorpholide), DTPA-bis(hydroxylated-amides), DOTA, DO3A, hydroxypropyl-DO3A, TETA, OTTA (1,4,7-triaza-10-oxacyclododecanetricarboxylic acid), EHPG, HIDA, PLED, DCTA and DCTP.

In addition to the aforementioned diagnostic imaging techniques, the present compositions can be used in a variety of therapeutic treatment modalities. For example, bioactive agents, for example, drugs, may be incorporated in the present compositions. Useful bioactive agents include, for example, statins, such as lovastatin, pravastatin, simvastatin, cerivastatin, fluvastatin, atrovastatin, eptastatin or mevastatin, antineoplastic agents, such as platinum compounds (e.g., spiroplatin, cisplatin, and carboplatin), methotrexate, adriamycin, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, mitotane, procarbazine hydrochloride dactinomycin (actinomycin D), daunorubicin hydrochloride, doxorubicin hydrochloride, taxol, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, etoposide (VP-16), interferon α-2a, interferon α-2b, teniposide (VM-26), vinblastine sulfate (VLB), vincristine sulfate, bleomycin, bleomycin sulfate, methotrexate, adriamycin, and arabinosyl; blood products such as parenteral iron, hemin, hematoporphyrins and their derivatives; biological response modifiers such as muramyldipeptide, muramyltripeptide, microbial cell wall components, lymphokines (e.g., bacterial endotoxin such as lipopolysaccharide, macrophage activation factor), sub-units of bacteria (such as Mycobacteria, Corynebacteria), the synthetic dipeptide N-acetyl-muramyl-L-alanyl-D-isoglutamine; anti-fungal agents such as ketoconazole, nystatin, griseofulvin, flucytosine (5-fc), miconazole, amphotericin B, ricin, and β-lactam antibiotics (e.g., sulfazecin); hormones such as growth hormone, melanocyte stimulating hormone, estradiol, beclomethasone dipropionate, betamethasone, betamethasone acetate and betamethasone sodium phosphate, vetamethasone disodium phosphate, vetamethasone sodium phosphate, cortisone acetate, dexamethasone, dexamethasone acetate, dexamethasone sodium phosphate, flunisolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide and fludrocortisone acetate; vitamins such as cyanocobalamin neinoic acid, retinoids and derivatives such as retinol palmitate, and a-tocopherol; peptides, such as manganese super oxide dismutase; enzymes such as alkaline phosphatase; anti-allergic agents such as amelexanox; anti-coagulation agents such as phenprocoumon and heparin; circulatory drugs such as propranolol; metabolic potentiators such as glutathione; antituberculars such as para-aminosalicylic acid, isoniazid, capreomycin sulfate cycloserine, ethambutol hydrochloride ethionamide, pyrazinamide, rifampin, and streptomycin sulfate; antivirals such as acyclovir, amantadine azidothymidine (AZT or Zidovudine), ribavirin and vidarabine monohydrate (adenine arabinoside, ara-A); antianginals such as diltiazem, nifedipine, verapamil, erythritol tetranitrate, isosorbide dinitrate, nitroglycerin (glyceryl trinitrate) and pentaerythritol tetranitrate; anticoagulants such as phenprocoumon, heparin; antibiotics such as dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin G, penicillin V, ticarcillin rifampin and tetracycline; antiinflammatories such as diflunisal, ibuprofen, indomethacin, meclofenamate, mefenamic acid, naproxen, oxyphenbutazone, phenylbutazone, piroxicam, sulindac, tolmetin, aspirin and salicylates; antiprotozoans such as chloroquine, hydroxychloroquine, metronidazole, quinine and meglumine antimonate; antirheumatics such as penicillamine; narcotics such as paregoric; opiates such as codeine, heroin, methadone, morphine and opium; cardiac glycosides such as deslanoside, digitoxin, digoxin, digitalin and digitalis; neuromuscular blockers such as atracurium mesylate, gallamine triethiodide, hexafluorenium bromide, metocurine iodide, pancuronium bromide, succinylcholine chloride (suxamethonium chloride), tubocurarine chloride and vecuronium bromide; sedatives (hypnotics) such as amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam and triazolam; local anesthetics such as bupivacaine hydrochloride, chloroprocaine hydrochloride, etidocaine hydrochloride, lidocaine hydrochloride, mepivacaine hydrochloride, procaine hydrochloride and tetracaine hydrochloride; general anesthetics such as droperidol, etomidate, fentanyl citrate with droperidol, ketamine hydrochloride, methohexital sodium and thiopental sodium; and radioactive particles or ions such as strontium, iodide rhenium and yttrium. Other suitable bioactive agents include the camptotheca alkaloids and derivatives thereof including, for example, camptothecin and ester or amide derivatives thereof, particularly at the 7, 9, 10, 11 and 20 ring positions, as well as irinotecan, topotecan and SN-38.

Additional bioactive agents which may be employed in the present compositions are disclosed in U.S. Pat. No. 5,770,222, the disclosure of which is hereby incorporated herein by reference, in its entirety.

Preferably, the bioactive agent employed in the present methods and compositions is a statin, with the statins lovastatin, pravastatin, simvastatin, cerivastatin, fluvastatin, atrovastatin, eptastatin and mevastatin being more preferred.

The lipid and/or vesicle compositions of the present invention may be prepared using any of a variety of suitable methods. These are described below separately for the embodiments involving lipid compositions and a gas, including gas filled vesicles, and embodiments involving lipid compositions and a gaseous precursor, including gaseous precursor filled vesicles, although compositions comprising both a gas and gaseous precursor form a part of the present invention.

A targeting ligand may be attached to the gas or gaseous precursor filled vesicle by bonding to one or more of the materials employed in the compositions from which they are made, including the lipids, polymers and/or auxiliary stabilizing materials, as described above.

A wide variety of methods are available for the preparation of the compositions, including vesicle compositions, such as micelles and/or liposomes. Included among these methods are, for example, shaling, drying, gas-installation, spray drying, and the like. Suitable methods for preparing vesicle compositions are described, for example, in U.S. Pat. No. 5,469,854, the disclosures of which are incorporated herein by reference. As noted above, the vesicles are preferably prepared from lipids which remain in the gel state.

With particular reference to the preparation of micelle compositions, the following discussion is provided. Micelles may be prepared using any one of a variety of conventional micellar preparatory methods which will be apparent to those skilled in the art. These methods typically involve suspension of the lipid compound in an organic solvent, evaporation of the solvent, resuspension in an aqueous medium, sonication and centrifugation. The foregoing methods, as well as others, are discussed, for example, in Canfield et al., Methods in Enzymology, Vol. 189, pp. 418-422 (1990); El-Gorab et al, Biochem. Biophys. Acta, Vol. 306, pp. 58-66 (1973); Colloidal Surfactant, Shinoda, K., Nakagana, Tamamushi and Isejura, Academic Press, NY (1963) (especially “The Formation of Micelles”, Shinoda, Chapter 1, pp. 1-88); Catalysis in Micellar and Macromolecular Systems, Fendler and Fendler, Academic Press, NY (1975). The disclosures of each of the foregoing publications are incorporated by reference herein, in their entirety.

As noted above, the vesicle composition may comprise liposomes. In any given liposome, the lipid compound(s) may be in the form of a monolayer or bilayer, and the mono- or bilayer lipids may be used to form one or more mono- or bilayers. In the case of more than one mono- or bilayer, the mono- or bilayers are generally concentric. Thus, the lipids may be used to form unilamellar liposomes (comprised of one monolayer or bilayer), oligolamellar liposomes (comprised of two or three monolayers or bilayers) or multilamellar liposomes (comprised of more than three monolayers or bilayers).

A wide variety of methods are available in connection with the preparation of liposome compositions. Accordingly, the liposomes may be prepared using any one of a variety of conventional liposomal preparatory techniques which will be apparent to those skilled in the art. These techniques include, for example, solvent dialysis, French press, extrusion (with or without freeze-thaw), reverse phase evaporation, simple freeze-thaw, sonication, chelate dialysis, homogenization, solvent infusion, microemulsification, spontaneous formation, solvent vaporization, solvent dialysis, French pressure cell technique, controlled detergent dialysis, and others, each involving the preparation of the vesicles in various fashions. See, e.g., Madden et al., Chemistry and Physics of Lipids, 1990 53, 37-46, the disclosures of which are hereby incorporated herein by reference in their entirety. Suitable freeze-thaw techniques are described, for example, in International Application Serial No. PCT/US89/05040, filed Nov. 8, 1989, the disclosures of which are incorporated herein by reference in their entirety. Methods which involve freeze-thaw techniques are preferred in connection with the preparation of liposomes. Preparation of the liposomes may be carried out in a solution, such as an aqueous saline solution, aqueous phosphate buffer solution, or sterile water. The liposomes may also be prepared by various processes which involve shaking or vortexing. This may be achieved, for example, by the use of a mechanical shaking device, such as a Wig-L-Bug™ (Crescent Dental, Lyons, Ill.), a Mixomat, sold by Degussa AG, Franfurt, Germany, a Capmix, sold by Espe Fabrik Pharmazeutischer Praeparate GMBH & Co., Seefeld, Oberay Germany, a Silamat Plus, sold by Vivadent, Lechtenstein, or a Vibros, sold by Quayle Dental, Sussex, England. Conventional microemulsification equipment, such as a Microfluidizer™ (Microfluidics, Woburn, Mass.) may also be used.

Spray drying may be also employed to prepare the gas-filled vesicles. Utilizing this procedure, the lipids may be pre-mixed in an aqueous environment and then spray dried to produce gas-filled vesicles. The vesicles may be stored under a headspace of a desired gas.

Many liposomal preparatory techniques which may be adapted for use in the preparation of vesicle compositions are discussed, for example, in U.S. Pat. No. 4,728,578; U.K. Patent Application GB 2193095 A; U.S. Pat. No. 4,728,575; U.S. Pat. No. 4,737,323; International Application Serial No. PCT/US85/01161; Mayer et al., Biochimica et Biophysica Acta, Vol. 858, pp. 161-168 (1986); Hope et al., Biochimica et Biophysica Acta, Vol. 812, pp. 55-65 (1985); U.S. Pat. No. 4,533,254; Mayhew et al., Methods in Enzymology, Vol. 149, pp. 64-77 (1987); Mayhew et al., Biochimica et Biophysica Acta, Vol 755, pp. 169-74 (1984); Cheng et al, Investigative Radiology, Vol. 22, pp. 47-55 (1987); International Application Serial No. PCT/US89/05040; U.S. Pat. No. 4,162,282; U.S. Pat. No. 4,310,505; U.S. Pat. No. 4,921,706; and Liposome Technology, Gregoriadis, G., ed., Vol. I, pp. 29-31, 51-67 and 79-108 (CRC Press Inc., Boca Raton, Fla. 1984), the disclosures of each of which are hereby incorporated by reference herein, in their entirety.

Lipid compositions comprising a gas can be prepared by agitating an aqueous solution containing, if desired, a stabilizing material, in the presence of a gas. The term “agitating,” as used herein, means any shaking motion of an aqueous solution such that gas is introduced from the local ambient environment into the aqueous solution. This agitation is preferably conducted at a temperature below the gel to liquid crystalline phase transition temperature of the lipid. The shaking involved in the agitation of the solutions is preferably of sufficient force to result in the formation of a lipid composition, including vesicle compositions, and particularly vesicle compositions comprising gas filled vesicles. The shaking may be by swirling, such as by vortexing, side-to-side, or up and down motion. Different types of motion may be combined. Also, the shaking may occur by shaking the container holding the aqueous lipid solution, or by shaking the aqueous solution within the container without shaking the container itself. The shaking may occur manually or by machine. Mechanical shakers that may be used include, for example, a shaker table such as a VWR Scientific (Cerritos, Calif.) shaker table, as well as any of the shaking devices described hereinbefore, with the Capmix (Espe Fabrik Pharmazeutischer Praeparate GMBH & Co., Seefeld, Oberay Germany) being preferred. It has been found that certain modes of shaking or vortexing can be used to make vesicles within a preferred size range. Shaking is preferred, and it is preferred that the shaking be carried out using the Espe Capmix mechanical shaker. In accordance with this preferred method, it is preferred that a reciprocating motion be utilized to generate the lipid compositions, and particularly vesicle compositions. It is even more preferred that the motion be reciprocating in the form of an arc. It is contemplated that the rate of reciprocation, as well as the arc thereof, is particularly important in connection with the formation of vesicles. Preferably, the number of reciprocations or full cycle oscillations is from about 1000 to about 20,000 per minute. More preferably, the number of reciprocations or oscillations is from about 2500 to about 8000, with reciprocations or oscillations of from about 3300 to about 5000 being even more preferred. Of course, the number of oscillations can be dependent upon the mass of the contents being agitated. Generally speaking, a larger mass requires fewer oscillations. Another means for producing shaking includes the action of gas emitted under high velocity or pressure.

It will also be understood that preferably, with a larger volume of aqueous solution, the total amount of force will be correspondingly increased. Vigorous shaking is defined as at least about 60 shaking motions per minute, and is preferred. Vortexing at about 60 to about 300 revolutions per minute is more preferred. Vortexing at about 300 to about 1800 revolutions per minute is even more preferred.

In addition to the simple shaking methods described above, more elaborate methods can also be employed. Such elaborate methods include, for example, liquid crystalline shaking gas instillation processes and vacuum drying gas instillation processes, such as those described in Unger, et al., U.S. Pat. No. 5,580,275, the disclosures of which are incorporated herein by reference, in their entirety. Although any of a number of varying techniques can be used, the vesicle compositions employed in the present invention are preferably prepared using a shaking technique. Preferably, the shaking technique involves agitation with a mechanical shaking apparatus, such as an Espe Capmix (Seefeld, Oberay Germany), using, for example, the techniques disclosed in Unger, et al., U.S. Pat. No. 5,542,935, the disclosures of which are hereby incorporated herein by reference in their entirety.

The size of gas filled vesicles can be adjusted, if desired, by a variety of procedures, including, for example, microemulsification, vortexing, extrusion, filtration, sonication, homogenization, repeated freezing and thawing cycles, extrusion under pressure through pores of defined size, and similar methods. Gas filled vesicles prepared in accordance with the methods described herein can range in size from less than about 1 μm to greater than about 100 μm. In addition, after extrusion and sterilization procedures, which are discussed in detail below, agitation or shaking provides vesicle compositions which provide substantially no or minimal residual anhydrous lipid phase in the remainder of the solution. (Bangham, A. D., Standish, M. M, & Watkins, J. C., J. Mol. Biol. Vol. 13, pp. 238-252 (1965). If desired, the vesicles of the present invention may be used as they are formed, without any attempt at further modification of the size thereof. For intravascular use, the vesicles preferably have diameters of less than about 30 μm, and more preferably, less than about 12 μm. For targeted intravascular use including, for example, binding to certain tissue, such as cancerous tissue, the vesicles can be significantly smaller, for example, less than about 100 nm in diameter. For enteric or gastrointestinal use, the vesicles can be significantly larger, for example, up to a millimeter in size. Preferably, the vesicles are sized to have diameters of from about 2 μm to about 100 μm.

The gas filled vesicles may be sized by a simple process of extrusion through filters wherein the filter pore sizes control the size distribution of the resulting gas filled vesicles. By using two or more cascaded or stacked set of filters, for example, a 10 μm filter followed by an 8 μm filter, the gas filled vesicles can be selected to have a very narrow size distribution around 7 to 9 μm. After filtration, these gas filled vesicles can remain stable for over 24 hours.

The sizing or filtration step may be accomplished by the use, for example, of a filter assembly when the composition is removed from a sterile vial prior to use, or more preferably, the filter assembly may be incorporated into a syringe during use. The method of sizing the vesicles will then comprise using a syringe comprising a barrel, at least one filter, and a needle; and will be carried out by a step of extracting which comprises extruding the vesicles from the barrel through the filter fitted to the syringe between the barrel and the needle, thereby sizing the vesicles before they are administered to a patient. The step of extracting may also comprise drawing the vesicles into the syringe, where the filter will function in the same way to size the vesicles upon entrance into the syringe. Another alternative is to fill such a syringe with vesicles which have already been sized by some other means, in which case the filter now functions to ensure that only vesicles within the desired size range, or of the desired maximum size, are subsequently administered by extrusion from the syringe.

In certain preferred embodiments, the vesicle compositions may be heat sterilized or filter sterilized and extruded through a filter prior to shaking. Generally speaking, vesicle compositions comprising a gas may be heat sterilized, and vesicle compositions comprising gaseous precursors may be filter sterilized. Once gas filled vesicles are formed, they may be filtered for sizing as described above. Performing these steps prior to the formation of gas and gaseous precursor filled vesicles provide sterile gas filled vesicles ready for administration to a patient. For example, a mixing vessel such as a vial or syringe may be filled with a filtered lipid composition, and the composition may be sterilized within the mixing vessel, for example, by autoclaving. Gas may be instilled into the composition to form gas filled vesicles by shaking the sterile vessel. Preferably, the sterile vessel is equipped with a filter positioned such that the gas filled vesicles pass through the filter before contacting a patient.

The step of extruding the solution of lipid compound through a filter decreases the amount of unhydrated material by breaking up any dried materials and exposing a greater surface area for hydration. Preferably, the filter has a pore size of about 0.1 to about 5 μm, more preferably, about 0.1 to about 4 μm, even more preferably, about 0.1 to about 2 μm, and still more preferably, about 1 μm. Unhydrated compound, which is generally undesirable, appears as amorphous clumps of non-uniform size.

The sterilization step provides a composition that may be readily administered to a patient for diagnostic imaging including, for example, ultrasound or CT. In certain preferred embodiments, sterilization may be accomplished by heat sterilization, preferably, by autoclaving the solution at a temperature of at least about 100° C., and more preferably, by autoclaving at about 100° C. to about 130° C., even more preferably, about 110° C. to about 130° C., still more preferably, about 120° C. to about 130° C., and even more preferably, about 130° C. Preferably, heating occurs for at least about 1 minute, more preferably, about 1 to about 30 minutes, even more preferably, about 10 to about 20 minutes, and still more preferably, about 15 minutes.

If desired, the extrusion and heating steps, as outlined above, may be reversed, or only one of the two steps can be used. Other modes of sterilization may be used, including, for example, exposure to gamma radiation.

In addition to the aforementioned embodiments, gaseous precursors contained in vesicles can be formulated which, upon activation, for example, by exposure to elevated temperature, varying pH, or light, undergo a phase transition from, for example, a liquid, including a liquid entrapped in a vesicle, to a gas, expanding to create the gas filled vesicles described herein. This technique is described in detail in Unger, et al., U.S. Pat. No. 5,542,935 and Unger et al., U.S. Pat. No. 5,585,112, the disclosures of which are incorporated herein by reference, in their entirety.

The preferred method of activating the gaseous precursor is by exposure to elevated temperature. Activation or transition temperature, and like terms, refer to the boiling point of the gaseous precursor and is the temperature at which the liquid to gaseous phase transition of the gaseous precursor takes place. Useful gaseous precursors are those materials which have boiling points in the range of about −100° C. to about 70° C. The activation temperature is particular to each gaseous precursor. An activation temperature of about 37° C., or about human body temperature, is preferred for gaseous precursors in the context of the present invention. Thus, in preferred form, a liquid gaseous precursor is activated to become a gas at about 37° C. or below. The gaseous precursor may be in liquid or gaseous phase for use in the methods of the present invention.

The methods of preparing the gaseous precursor filled vesicles may be carried out below the boiling point of the gaseous precursor such that a liquid is incorporated, for example, into a vesicle. In addition, the methods may be conducted at the boiling point of the gaseous precursor, such that a gas is incorporated, for example, into a vesicle. For gaseous precursors having low temperature boiling points, liquid precursors may be emulsified using a microfluidizer device chilled to a low temperature. The boiling points may also be depressed using solvents in liquid media to utilize a precursor in liquid form. Further, the methods may be performed where the temperature is increased throughout the process, whereby the process starts with a gaseous precursor as a liquid and ends with a gas.

The gaseous precursor may be selected so as to form the gas in situ in the targeted tissue or fluid, in vivo upon entering the patient or animal, prior to use, during storage, or during manufacture. The methods of producing the temperature-activated gaseous precursor filled vesicles may be carried out at a temperature below the boiling point of the gaseous precursor. In this embodiment, the gaseous precursor is entrapped within a vesicle such that the phase transition does not occur during manufacture. Instead, the gaseous precursor filled vesicles are manufactured in the liquid phase of the gaseous precursor. Activation of the phase transition may take place at any time as the temperature is allowed to exceed the boiling point of the precursor. Also, knowing the amount of liquid in a droplet of liquid gaseous precursor, the size of the vesicles upon attaining the gaseous state may be determined.

Alternatively, the gaseous precursors may be utilized to create stable gas filled vesicles which are pre-formed prior to use. In this embodiment, the gaseous precursor is added to a container housing a lipid composition at a temperature below the liquid-gaseous phase transition temperature of the respective gaseous precursor. As the temperature is increased, and an emulsion is formed between the gaseous precursor and liquid solution, the gaseous precursor undergoes transition from the liquid to the gaseous state. As a result of this heating and gas formation, the gas displaces the air in the head space above the liquid mixture so as to form gas filled vesicles which entrap the gas of the gaseous precursor, ambient gas (e.g. air), or coentrap gas state gaseous precursor and ambient air. This phase transition can be used for optimal mixing and formation of the contrast agent. For example, the gaseous precursor, perfluorobutane, can be entrapped in the lipid vesicles and as the temperature is raised beyond the boiling point of perfluorobutane (4° C.), perfluorobutane gas is entrapped in the vesicles.

Accordingly, the gaseous precursors may be selected to form gas filled vesicles in vivo or may be designed to produce the gas filled vesicles in situ, during the manufacturing process, on storage, or at some time prior to use. A water bath, sonicator or hydrodynamic activation by pulling back the plunger of a syringe against a closed stopcock may be used to activate targeted gas-filled vesicles from temperative-sensitive gaseous precursors prior to I.V. injection.

As a further embodiment of this invention, by pre-forming the gaseous precursor in the liquid state into an aqueous emulsion, the maximum size of the vesicle may be estimated by using the ideal gas law, once the transition to the gaseous state is effectuated. For the purpose of making gas filled vesicles from gaseous precursors, the gas phase is assumed to form instantaneously and substantially no gas in the newly formed vesicle has been depleted due to diffusion into the liquid, which is generally aqueous in nature. Hence, from a known liquid volume in the emulsion, one would be able to predict an upper limit to the size of the gas filled vesicle.

In embodiments of the present invention, a mixture of a lipid compound and a gaseous precursor, containing liquid droplets of defined size, may be formulated such that upon reaching a specific temperature, for example, the boiling point of the gaseous precursor, the droplets will expand into gas filled vesicles of defined size. The defined size represents an upper limit to the actual size because the ideal gas law cannot account for such factors as gas diffusion into solution, loss of gas to the atmosphere, and the effects of increased pressure.

The ideal gas law, which can be used for calculating the increase in the volume of the gas bubbles upon transitioning from liquid to gaseous states, is as follows:
PV=nRT
where
P is pressure in atmospheres (atm);
V is volume in liters (L);
n is moles of gas;
T is temperature in degrees Kelvin (K); and

R is the ideal gas constant (22.4 L-atm/K-mole).

With knowledge of volume, density, and temperature of the liquid in the mixture of liquids, the amount, for example, in moles, and volume of liquid precursor may be calculated which, when converted to a gas, will expand into a vesicle of known volume. The calculated volume will reflect an upper limit to the size of the gas filled vesicle, assuming instantaneous expansion into a gas filled vesicle and negligible diffusion of the gas over the time of the expansion.

Thus, for stabilization of the precursor in the liquid state in a mixture wherein the precursor droplet is spherical, the volume of the precursor droplet may be determined by the equation:
Volume (spherical vesicle)=4/3=πr³
where
r is the radius of the sphere.
   Thus, once the volume is predicted, and knowing the density of the liquid at the desired temperature, the amount of liquid gaseous precursor in the droplet may be determined. In more descriptive terms, the following can be applied:
   V_{gas}=4/3π(r_{gas})³
   by the ideal gas law,
   PV=nRT
   substituting reveals,
   V_{gas}=nRT/P_{gas}
   or,
(A) n=4/3 [πr_{gas}³] P/RT
amount n=4/3 [πr_{gas}³ P/RT]·MWₙ
Converting back to a liquid volume
(B) V_{liq}=[4/3 [πr_{gas}³]P/RT]·MWₙ/D]
   where D is the density of the precursor.

Solving for the diameter of the liquid droplet,
(C) diameter/2=[¾π[4/3·[πr_{gas}³] P/RT] MWₙ/D]^{1/3}
   which reduces to
   Diameter=2[[rg³] P/RT [MWn/D]]11.

As a further means of preparing vesicles of, the desired size for use in the methods of the present invention, and with a knowledge of the volume and especially the radius of the liquid droplets, one can use appropriately sized filters to size the gaseous precursor droplets to the appropriate diameter sphere.

A representative gaseous precursor may be used to form a vesicle of defined size, for example, 10 μm diameter. In this example, the vesicle is formed in the bloodstream of a human being, thus the typical temperature would be 37° C. or 310 K. At a pressure of 1 atmosphere and using the equation in (A), 7.54×10⁻¹⁷ moles of gaseous precursor would be required to fill the volume of a 10 μm diameter vesicle.

Using the above calculated amount of gaseous precursor and 1-fluorobutane, which possesses a molecular weight of 76.11, a boiling point of 32.5° C. and a density of 0.7789 g/mL at 20° C., further calculations predict that 5.74×10⁻¹⁵ grams of this precursor would be required for a 10 μm vesicle. Extrapolating further, and with the knowledge of the density, equation (B) further predicts that 8.47×10⁻¹⁶ mL of liquid precursor is necessary to form a vesicle with an upper limit of 10 μm.

Finally, using equation (C), a mixture, for example, an emulsion containing droplets with a radius of 0.0272 μm or a corresponding diameter of 0.0544 μm, is formed to make a gaseous precursor filled vesicle with an upper limit of a 10 μm vesicle.

An emulsion of this particular size could be easily achieved by the use of an appropriately sized filter. In addition, as seen by the size of the filter necessary to form gaseous precursor droplets of defined size, the size of the filter would also suffice to remove any possible bacterial contaminants and, hence, can be used as a sterile filtration as well.

This embodiment for preparing gas filled vesicles may be applied to all gaseous precursors activated by temperature. In fact, depression of the freezing point of the solvent system allows the use of gaseous precursors which would undergo liquid-to-gas phase transitions at temperatures below 0° C. The solvent system can be selected to provide a medium for suspension of the gaseous precursor. For example, 20% propylene glycol miscible in buffered saline exhibits a freezing point depression well below the freezing point of water alone. By increasing the amount of propylene glycol or adding materials such as sodium chloride, the freezing point can be depressed even further.

The selection of appropriate solvent systems may be determined by physical methods as well. When substances, solid or liquid, herein referred to as solutes, are dissolved in a solvent, such as water based buffers, the freezing point is lowered by an amount that is dependent upon the composition of the solution. Thus, as defined by Wall, one can express the freezing point depression of the solvent by the following equation:
Inxₐ=In(1−x_{b})=ΔH_{fur}/R(1/Tₒ−1/T)
where
xₐ is the mole fraction of the solvent;
x_{b} is the mole fraction of the solute;
ΔH_{fus} is the heat of fusion of the solvent; and

Tₒ is the normal freezing point of the solvent.

The normal freezing point of the solvent can be obtained by solving the equation. If x_{b} is small relative to xₐ, then the above equation may be rewritten as follows.
x^{b}=ΔH_{fus}/R[T−Tₒ/TₒT]=ΔH_{fus}ΔT/RTₒ²
The above equation assumes the change in temperature ΔT is small compared to T₂. This equation can be simplified further by expressing the concentration of the solute in terms of molality, m (moles of solute per thousand grams of solvent). Thus, the equation can be rewritten as follows.
X_{b}=m/[m+1000/mₐ]=mMa/1000
where Ma is the molecular weight of the solvent.

Thus, substituting for the fraction x_{b}:
ΔT=[MₐRTₒ²/1000ΔH_{fus}]m
or
ΔT=K_{f}m, where
K_{f}=MₐRTₒ²/1000ΔHf_{fus}
K_{f} is the molal freezing point and is equal to 1.86 degrees per unit of molal concentration for water at one atmosphere pressure. The above equation may be used to accurately determine the molal freezing point of solutions of gaseous-precursor filled vesicles. Accordingly, the above equation can be applied to estimate freezing point depressions and to determine the appropriate concentrations of liquid or solid solute necessary to depress the solvent freezing temperature to an appropriate value.

Methods of preparing the temperature activated gaseous precursor filled vesicles include:
(a) vortexing and/or shaking an aqueous mixture of gaseous precursor and additional materials as desired, including, for example, stabilizing materials, thickening agents and/or dispersing agents. Optional variations of this method include autoclaving before vortexing or shaking; heating an aqueous mixture of gaseous precursor; venting the vessel containing the mixture/suspension; shaking or permitting the gaseous precursor filled vesicle to form spontaneously and cooling down the suspension of gaseous precursor filled vesicles; and extruding an aqueous suspension of gaseous precursor through a filter of about 0.22 μm. Alternatively, filtering may be performed during in vivo administration of the vesicles such that a filter of about 0.22 μm is employed;
(b) microemulsification whereby an aqueous mixture of gaseous precursor is emulsified by agitation and heated to form, for example, vesicles prior to administration to a patient;
(c) heating a gaseous precursor in a mixture, with or without agitation, whereby the less dense gaseous precursor filled vesicles float to the top of the solution by expanding and displacing other vesicles in the vessel and venting the vessel to release air; and
(d) utilizing in any of the above methods a sealed vessel to hold the aqueous suspension of gaseous precursor and maintaining the suspension at a temperature below the phase transition temperature of the gaseous precursor, followed by autoclaving to raise the temperature above the phase transition temperature, optionally with shaking, or permitting the gaseous precursor vesicle to form spontaneously, whereby the expanded gaseous precursor in the sealed vessel increases the pressure in the vessel, and cooling down the gas filled vesicle suspension, after which shaking may also take place.

Freeze drying is useful to remove water and organic materials prior to the shaking installation method. Drying installation methods may be used to remove water from vesicles. By pre-entrapping the gaseous precursor in the dried vesicles (i.e. prior to drying) after warming, the gaseous precursor may expand to fill the vesicle. Gaseous precursors can also be used to fill dried vesicles after they have been subjected to vacuum. As the dried vesicles are kept at a temperature below their gel state to liquid crystalline temperature, the drying chamber can be slowly filled with the gaseous precursor in its gaseous state. For example, perfluorobutane can be used to fill dried vesicles at temperatures above 4° C. (the boiling point of perfluorobutane).

Preferred methods for preparing the temperature activated gaseous precursor filled vesicles comprise shaking an aqueous solution having a lipid compound in the presence of a gaseous precursor at a temperature below the liquid state to gas state phase transition temperature of the gaseous precursor. This is preferably conducted at a temperature below the gel state to liquid crystalline state phase transition temperature of the lipid. The mixture is then heated to a temperature above the liquid state to gas state phase transition temperature of the gaseous precursor which causes the precursor to volatilize and expand. Heating is then discontinued, and the temperature of the mixture is then allowed to drop below the liquid state to gas state phase transition temperature of the gaseous precursor. Shaking of the mixture may take place during the heating step, or subsequently after the mixture is allowed to cool.

Other methods for preparing gaseous precursor filled vesicles can involve shaking an aqueous solution of, for example, a lipid and a gaseous precursor, and separating the resulting gaseous precursor filled vesicles.

Conventional, aqueous-filled liposomes of the prior art are routinely formed at a temperature above the phase transition temperature of the lipids used to make them, since they are more flexible and thus useful in biological systems in the liquid crystalline state. See, for example, Szoka and Papahadjopoulos, Proc. Natl. Acad. Sci. 1978, 75, 4194-4198. In contrast, the vesicles made according to certain preferred embodiments described herein are gaseous precursor filled, which imparts greater flexibility, since gaseous precursors after gas formation are more compressible and compliant than an aqueous solution.

The methods contemplated by the present invention provide for shaking an aqueous solution comprising a lipid, in the presence of a temperature activatable gaseous precursor. Preferably, the shaking is of sufficient force such that a foam is formed within a short period of time, such as about 30 minutes, and preferably within about 20 minutes, and more preferably, within about 10 minutes. The shaking may involve microemulsifying, microfluidizing, swirling (such as by vortexing), side-to-side, or up and down motion. In the case of the addition of gaseous precursor in the liquid state, sonication may be used in addition to the shaking methods set forth above. Further, different types of motion may be combined. Also, the shaking may occur by shaking the container holding the aqueous lipid solution, or by shaking the aqueous solution within the container without shaking the container itself Further, the shaking may occur manually or by machine. Mechanical shakers that may be used include, for example, the mechanical shakers described hereinbefore, with an Espe Capmix (Seefeld, Oberay Germany) being preferred. Another means for producing shaking includes the action of gaseous precursor emitted under high velocity or pressure.

According to the methods described herein, a gas, such as air, may also be provided by the local ambient atmosphere. The local ambient atmosphere can include the atmosphere within a sealed container, as well as the external environment. Alternatively, for example, a gas may be injected into or otherwise added to the container having the aqueous lipid solution or into the aqueous lipid solution itself to provide a gas other than air. Gases that are lighter than air are generally added to a sealed container, while gases heavier than air can be added to a sealed or an unsealed container. Accordingly, the present invention includes co-entrapment of air and/or other gases along with gaseous precursors.

Hence, the gaseous precursor filled vesicles can be used in substantially the same manner as the gas filled vesicles described herein, once activated by application to the tissues of a host, where such factors as temperature or pH may be used to cause generation of the gas. It is preferred that the gaseous precursors undergo phase transitions from liquid to gaseous states at near the normal body temperature of the host, and are thereby activated, for example, by the in vivo temperature of the host so as to undergo transition to the gaseous phase therein. Alternating, activation prior to I.V. injection may be used, for example, by thermal, mechanical or optical means. This activation can occur where, for example, the host tissue is human tissue having a normal temperature of about 37° C. and the gaseous precursors undergo phase transitions from liquid to gaseous states near 37° C.

As noted above, the lipid and/or vesicle compositions may be sterilized by autoclave or sterile filtration if these processes are performed before the installation step or prior to temperature mediated conversion of the temperature sensitive gaseous precursors within the compositions. Alternatively, one or more anti-bactericidal agents and/or preservatives may be included in the formulation of the compositions, such as sodium benzoate, quaternary ammonium salts, sodium azide, methyl paraben, propyl paraben, sorbic acid, ascorbylpaimitate, butylated hydroxyanisole, butylated hydroxytoluene, chlorobutanol, dehydroacetic acid, ethylenediamine, monothioglycerol, potassium benzoate, potassium metabisulfite, potassium sorbate, sodium bisulfite, sulfur dioxide, and organic mercurial salts. Such sterilization, which may also be achieved by other conventional means, such as by irradiation, will be necessary where the stabilized vesicles are used for imaging under invasive circumstances, e.g., intravascularly or intraperitonealy. The appropriate means of sterilization will be apparent to the artisan based on the present disclosure.

Vesicle compositions which comprise vesicles formulated from polymers may be prepared by various processes, as will be readily apparent to those skilled in the art, once armed with the present disclosure. Exemplary processes include, for example, interfacial polymerization, phase separation and coacervation, multiorifice centrifugal preparation, and solvent evaporation. Suitable procedures which may be employed or modified in accordance with the present disclosure to prepare vesicles from polymers include those procedures disclosed in Garner et al., U.S. Pat. No. 4,179,546, Garner, U.S. Pat. No. 3,945,956, Cohrs et al., U.S. Pat. No. 4,108,806, Japan Kokai Tokkyo Koho 62 286534, British Patent No. 1,044,680, Kenaga et al., U.S. Pat. No. 3,293,114, Morehouse et al., U.S. Pat. No. 3,401,475, Walters, U.S. Pat. No. 3,479,811, Walters et al., U.S. Pat. No. 3,488,714, Morehouse et al., U.S. Pat. No. 3,615,972, Baker et al., U.S. Pat. No. 4,549,892, Sands et al., U.S. Pat. No. 4,540,629, Sands et al., U.S. Pat. No. 4,421,562, Sands, U.S. Pat. No. 4,420,442, Mathiowitz et al., U.S. Pat. No. 4,898,734, Lencki et al., U.S. Pat. No. 4,822,534, Herbig et al., U.S. Pat. No. 3,732,172, Himmel et al., U.S. Pat. No. 3,594,326, Sommerville et al., U.S. Pat. No. 3,015,128, Deasy, Microencapsulation and Related Drug Processes, Vol. 20, Chs. 9 and 10, pp. 195-240 (Marcel Dekker, Inc., N.Y., 1984), Chang et al., Canadian J. of Physiology and Pharmacology, Vol 44, pp. 115-129 (1966), and Chang, Science, Vol. 146, pp. 524-525 (1964), the disclosures of each of which are incorporated herein by reference in their entirety.

In accordance with a preferred synthesis protocol, the vesicles may be prepared using a heat expansion process, such as, for example, the process described in Garner et al., U.S. Pat. No. 4,179,546, Garner, U.S. Pat. No. 3,945,956, Cohrs et al., U.S. Pat. No. 4,108,806, British Patent No. 1,044,680, and Japan Kokai Tokkyo Koho 62 286534. In general terms, the heat expansion process may be carried out by preparing vesicles of an expandable polymer or copolymer which may contain in their void (cavity) a volatile liquid (gaseous precursor). The vesicle is then heated, plasticising the vesicle and converting the volatile liquid into a gas, causing the vesicle to expand to up to about several times its original size. When the heat is removed, the thermoplastic polymer retains at least some of its expanded shape. Vesicles produced by this process tend to be of particularly low density, and are thus preferred. The foregoing described process is well known in the art, and may be referred to as the heat expansion process for preparing low density vesicles.

Polymers useful in the heat expansion process will be readily apparent to those skilled in the art and include thermoplastic polymers or copolymers, including polymers or copolymers of many of the monomers described above. Preferable of the polymers and copolymers described above include the following copolymers: polyvinylidene-polyacrylonitrile, polyvinylidene-polyacrylonitrile-polymethyl-methacrylate, and polystyrene-polyacrylonitrile. A most preferred copolymer is polyvinylidene-polyacrylonitrile.

Volatile liquids useful in the heat expansion process will also be well known to those skilled in the art and include: aliphatic hydrocarbons such as ethane, ethylene, propane, propene, butane, isobutane, neopentane, acetylene, hexane, heptane; chlorofluorocarbons such as CCl₃F, CCl₂F₃, CClF₃, CClF₂—CCl₂F₂, chloroheptafluoro-cyclobutane, and 1,2-dichlorohexafluorocyclobutane; tetraalkyl silanes, such as tetramethyl silane, trimethylethyl silane, trimethylisopropyl silane, and trimethyl n-propyl silane; as well as perfluorocarbons, including the perfluorocarbons described above. In general, it is important that the volatile liquid not be a solvent for the polymer or copolymer being utilized. It is also preferred that the volatile liquid have a boiling point that is below the softening point of the involved polymer or co-polymer. Boiling points of various volatile liquids and softening points of various polymers and copolymers will be readily ascertainable to one skilled in the art, and suitable combinations of polymers or copolymers and volatile liquids will be easily apparent to the skilled artisan. By way of guidance, and as one skilled in the art would recognize, generally as the length of the carbon chain of the volatile liquid increases, the boiling point of that liquid increases also. Also, mildly preheating the vesicles in water in the presence of hydrogen peroxide prior to definitive heating and expansion may pre-soften the vesicle to allow expansion to occur more readily.

For example, to produce vesicles from synthetic polymers, vinylidene and acrylonitrile may be copolymerized in a medium of isobutane liquid using one or more of the foregoing modified or unmodified literature procedures, such that isobutane becomes entrapped within the vesicles. When such vesicles are then heated to a temperature of from about 80° C. to about 120° C., the isobutane gas expands, which in turn expands the vesicles. After heat is removed, the expanded polyvinylidene and acrylo-nitrile copolymer vesicles remain substantially fixed in their expanded position. The resulting low density vesicles are extremely stable both dry and suspended in an aqueous media. Isobutane is utilized herein merely as an illustrative liquid, with the understanding that other liquids which undergo liquid/gas transitions at temperatures useful for the synthesis of these vesicles and formation of the very low density vesicles upon heating can be substituted for isobutane. Similarly, monomers other than vinylidene and acrylonitrile may be employed in preparing the vesicles.

In certain preferred embodiments, the vesicles which are formulated from synthetic polymers and which may be employed in the methods of the present invention are commercially available from Expancel, Nobel Industries (Sundsvall, Sweden), including EXPANCEL 551 DE™ microspheres. The EXPANCEL 551 DE™ microspheres are composed of a copolymer of vinylidene and acrylonitrile which have encapsulated therein isobutane liquid. Such microspheres are sold as a dry composition and are approximately 50 microns in size. The EXPANCEL 551 DE™ microspheres have a specific gravity of only 0.02 to 0.05, which is between one-fiftieth and one-twentieth the density of water.

In any of the techniques described above for the preparation of polymer-based vesicles, the targeting ligands may be incorporated with the polymers before, during or after formation of the vesicles, as would be apparent to one of ordinary skill in the art, once armed with the present disclosure.

As with the preparation of lipid and/or vesicle compositions, a wide variety of techniques are available for the preparation of lipid formulations. For example, the lipid and/or vesicle formulations may be prepared from a mixture of lipid compounds, bioactive agent and gas or gaseous precursor. In this case, lipid compositions are prepared as described above in which the compositions also comprise bioactive agent. Thus, for example, micelles can be prepared in the presence of a bioactive agent. In connection with lipid compositions which comprise a gas, the preparation can involve, for example, bubbling a gas directly into a mixture of the lipid compounds and one or more additional materials. Alternatively, the lipid compositions may be preformed from lipid compounds and gas or gaseous precursor. In the latter case, the bioactive agent is then added to the lipid composition prior to use. For example, an aqueous mixture of liposomes and gas may be prepared to which the bioactive agent is added and which is agitated to provide the liposome formulation. The liposome formulation can be readily isolated since the gas and/or bioactive agent filled liposome vesicles generally float to the top of the aqueous solution. Excess bioactive agent can be recovered from the remaining aqueous solution.

As those skilled in the art will recognize, any of the lipid and/or vesicle compositions and/or lipid and/or vesicle formulations may be lyophilized for storage, and reconstituted, for example, with an aqueous medium (such as sterile water, phosphate buffered solution, or aqueous saline solution), with the aid of vigorous agitation. To prevent agglutination or fusion of the lipids and/or vesicles as a result of lyophilization, it may be useful to include additives which prevent such fusion or agglutination from occurring. Additives which may be useful include sorbitol, mannitol, sodium chloride, glucose, trehalose, polyvinylpyrrolidone and poly(ethylene glycol) (PEG), for example, PEG 400. These and other additives are described in the literature, such as in the U.S. Pharmacopeia, USP XXII, NF XVII, The United States Pharmacopeia, The National Formulary, United States Pharmacopeial Convention Inc., 12601 Twinbrook Parkway, Rockville, Md. 20852, the disclosures of which are hereby incorporated herein by reference in their entirety. Lyophilized preparations generally have the advantage of greater shelf life.

As discussed above, the compositions of the present invention, including gas and/or gaseous precursor filled vesicles, are useful as contrast agents for diagnostic imaging, including, for example, ultrasound imaging (US), computed tomography (CT) imaging, including CT angiography (CTA) imaging, magnetic resonance (MR) imaging, magnetic resonance angiography (MRA), nuclear medicine, optical imaging and elastography.

In accordance with the present invention, there are provided methods of imaging one or more regions of a patient. The present invention also provides methods for diagnosing the presence or absence of diseased tissue in a patient. The methods of the present invention involve the administration of a contrast medium, in the form of a lipid and/or vesicle composition, to a patient. The patient is scanned using diagnostic imaging including, for example ultrasound imaging, to obtain visible images of an internal region of a patient. The methods are especially useful in providing images of the heart region, and to thereby detect and/or characterize the presence of a vascular plaque. The present methods can also be used in connection with the delivery of a bioactive agent to an internal region of a patient.

As one skilled in the art would recognize, administration of the lipid and/or vesicle compositions of the present invention can be carried out in various fashions, namely, parenterally, orally, or intraperitoneally. Parenteral administration, which is preferred, includes administration by the following routes: intravenous; intramuscular; interstitially; intra-arterially; intracavitary; subcutaneous; intraocular; intrasynovial; transepithelial, including transdermal; pulmonary via inhalation; ophthalmic; sublingual and buccal; topically, including ophthalmic; dermal; ocular; rectal; and nasal inhalation via insufflation. Intravenous administration is preferred among the routes of parenteral administration. The useful dosage to be administered and the particular mode of administration will vary depending upon the age, weight and the particular mammal and region thereof to be scanned, and the particular contrast agent employed. Typically, dosage is initiated at lower levels and increased until the desired contrast enhancement is achieved. Various combinations of the lipid compositions may be used to alter properties as desired, including viscosity, osmolarity or palatability. In carrying out the imaging methods of the present invention, the contrast medium can be used alone, or in combination with diagnostic, therapeutic or other agents. Such other agents include excipients such as flavoring or coloring materials. CT imaging techniques which are employed are conventional and are described, for example, in Computed Body Tomography, Lee, J. K. T., Sagel, S. S., and Stanley, R. J., eds., 1983, Ravens Press, New York, N.Y., especially the first two chapters thereof entitled “Physical Principles and Instrumentation”, Ter-Pogossian, M. M., and “Techniques”, Aronberg, D. J., the disclosures of which are incorporated by reference herein in their entirety.

In the case of diagnostic applications, ultrasonic energy is applied to at least a portion of the patient to image the target tissue. A visible image of an internal region of the patient is then obtained, such that the presence or absence of diseased tissue can be ascertained. With respect to ultrasound, ultrasonic imaging techniques, including second harmonic imaging, and gated imaging, are well known in the art, and are described, for example, in Uhlendorf, “Physics of Ultrasound Contrast Imaging: Scattering in the Linear Range”, IEEE Transactions on Ultrasonics, Ferroelectrics, and Frequency Control, Vol. 14(1), pp. 70-79 (1994) and Sutherland, et al., “Color Doppler Myocardial Imaging: A New Technique for the Assessment of Myocardial Function”, Journal of the American Society of Echocardiography, Vol. 7(5), pp. ⁴⁴1-458 (1994), the disclosures of which are hereby incorporated herein by reference in their entirety. The ultrasound may be performed using conventional probes, e.g. transcutaneous, extra-corporeal ultrasound or using catheter mounted transducers, e.g. from an intra-arterial approach. The frequency of the ultrasound may range from about 20 KiloHertz to about 100 megaHertz (and all combinations and subcombinations of ranges and specific values therein), but more preferably will vary from about 500 kiloHertz to about 30 MegaHertz, and even more preferably from about 1 MegaHertz to up to about 20 MegaHertz. Gray scale, color Doppler, Power Doppler, pulse inversion Doppler and other ultrasound techniques may be employed. In general, the contrast agent will enhance visibility of plaques that are diseased and active, particularly those in which inflammatory cells are active.

Ultrasound can be used for both diagnostic and therapeutic purposes. In diagnostic ultrasound, ultrasound waves or a train of pulses of ultrasound may be applied with a transducer. The ultrasound is generally pulsed rather than continuous, although it may be continuous, if desired. Thus, diagnostic ultrasound generally involves the application of a pulse of echoes, after which, during a listening period, the ultrasound transducer receives reflected signals. Harmonics, ultraharmonics or subharmonics may be used. The second harmonic mode may be beneficially employed, in which the 2×frequency is received, where x is the incidental frequency. This may serve to decrease the signal from the background material and enhance the signal from the transducer using the targeted contrast media of the present invention which may be targeted to the desired site, for example, blood clots. Other harmonics signals, such as odd harmonics signals, for example, 3x or 5x, would be similarly received using this method. Subharmonic signals, for example, x/2 and x/3, may also be received and processed so as to form an image.

A novel and particularly advantageous feature afforded with the present invention is the use of ultrasound technology not only for the detection of vascular plaques, but for the treatment of plaques as well. For example, in embodiments involving gas filled vesicles, the ultrasound energy may be amplified and concentrated in the plaque. This amplification and concentration of ultrasound energy may be used to dissolve the plaque. Generally speaking, this may be accomplished by applying pulses of ultrasound energy at the appropriate energy and frequency.

In addition to the pulsed method, continuous wave ultrasound, for example, Power Doppler, may be applied. This may be particularly useful where rigid vesicles, for example, vesicles formulated from polymethyl methacrylate, are employed. In this case, the relatively higher energy of the Power Doppler may be made to resonate the vesicles and thereby promote their rupture. This can create acoustic emissions which may be in the subharmonic or ultraharmonic range or, in some cases, in the same frequency as the applied ultrasound. It is contemplated that there will be a spectrum of acoustic signatures released in this process and the transducer so employed may receive the acoustic emissions to detect, for example, the presence of a clot. In addition, the process of vesicle rupture may be employed to transfer kinetic energy to the surface, for example of a plaque to promote dissolution of the plaque and/or to release a bioactive agent, particularly a statin. Thus, therapeutic treatment may be achieved during a combination of diagnostic and therapeutic ultrasound. Spectral Doppler may also be employed. In general, the levels of energy from diagnostic ultrasound are insufficient to promote the rupture of vesicles and to facilitate release and cellular uptake of the bioactive agents. As noted above, diagnostic ultrasound may involve the application of one or more pulses of sound. Pauses between pulses permits the reflected sonic signals to be received and analyzed. The limited number of pulses used in diagnostic ultrasound limits the effective energy which is delivered to the tissue that is being studied.

Higher energy ultrasound, for example, ultrasound which is generated by therapeutic ultrasound equipment, is generally capable of causing rupture of the vesicle species. In general, devices for therapeutic ultrasound employ from about 10 to about 100% duty cycles, depending on the area of tissue to be treated with the ultrasound. Areas of the body which are generally characterized by larger amounts of muscle mass, for example, backs and thighs, as well as highly vascularized tissues, such as heart tissue, may require a larger duty cycle, for example, up to about 100%.

In therapeutic ultrasound, continuous wave ultrasound is used to deliver higher energy levels. For the rupture of vesicles, continuous wave ultrasound is preferred, although the sound energy may be pulsed also. If pulsed sound energy is used, the sound will generally be pulsed in echo train lengths of from about 8 to about 20 or more pulses at a time. Preferably, the echo train lengths are about 20 pulses at a time. In addition, the frequency of the sound used may vary from about 0.025 to about 100 megahertz (MHz). In general, frequency for therapeutic ultrasound preferably ranges between about 0.75 and about 3 MHz, with from about 1 and about 2 MHz being more preferred. In addition, energy levels may vary from about 0.5 Watt (W) per square centimeter (cm²) to about 5.0 W/cm², with energy levels of from about 0.5 to about 2.5 W/cm² being preferred. Energy levels for therapeutic ultrasound involving hyperthermia are generally from about 5 W/cm² to about 50 W/cm². For very small vesicles, for example, vesicles having a diameter of less than about 0.5 μm, higher frequencies of sound are generally preferred. This is because smaller vesicles are capable of absorbing sonic energy more effectively at higher frequencies of sound. When very high frequencies are used, for example, greater than about 10 MHz, the sonic energy will generally penetrate fluids and tissues to a limited depth only. Thus, external application of the sonic energy may be suitable for skin and other superficial tissues. However, it is generally necessary for deep structures to focus the ultrasonic energy so that it is preferentially directed within a focal zone. Alternatively, the ultrasonic energy may be applied via interstitial probes, intravascular ultrasound catheters or endoluminal catheters. Such probes or catheters may be used, for example, in the esophagus for the diagnosis and/or treatment of esophageal carcinoma. In addition to the therapeutic uses discussed above, the present compositions can be employed in connection with esophageal carcinoma or in the coronary arteries for the treatment of atherosclerosis, as well as the therapeutic uses described, for example, in U.S. Pat. No. 5,149,319, the disclosures of which are hereby incorporated herein by reference, in their entirety.

A therapeutic ultrasound device may be used which employs two frequencies of ultrasound. The first frequency may be x, and the second frequency may be 2x. In preferred form, the device would be designed such that the focal zones of the first and second frequencies converge to a single focal zone. The focal zone of the device may then be directed to the targeted compositions, for example, targeted vesicle compositions, within the targeted tissue. This ultrasound device may provide second harmonic therapy with simultaneous application of the x and 2×frequencies of ultrasound energy. It is contemplated that, in the case of ultrasound involving vesicles, this second harmonic therapy may provide improved rupturing of vesicles as compared to ultrasound energy involving a single frequency. Also, it is contemplated that the preferred frequency range may reside within the fundamental harmonic frequencies of the vesicles. Lower energy may also be used with this device. An ultrasound device which may be employed in connection with the aforementioned second harmonic therapy is described, for example, in Kawabata, K. et al., Ultrasonics Sonochemistry, Vol. 3, pp. 1-5 (1996), the disclosures of which are hereby incorporated herein by reference, in their entirety.

For use with laser light, or other appropriate light source, and employing photosenstizing agents, the methods and compositions of the present invention may also be used to perform photoablative therapy of the plaques.

The concentration of lipid required to form a desired stabilized vesicle level will vary depending upon the type of lipid used, and may be readily determined by routine experimentation. For example, in preferred embodiments, the concentration of 1,2-dipalmitoylphosphatidylcholine (DPPC) used to form stabilized vesicles according to the methods of the present invention is about 0.1 mg/ml to about 30 mg/ml of saline solution, more preferably from about 0.5 mg/ml to about 20 mg/ml of saline solution, and most preferably from about 1 mg/ml to about 10 mg/ml of saline solution. The concentration of distearoylphosphatidylcholine (DSPC) used in preferred embodiments is about 0.1 mg/ml to about 30 mg/ml of saline solution, more preferably from about 0.5 mg/ml to about 20 mg/ml of saline solution, and most preferably from about 1 mg/ml to about 10 mg/ml of saline solution. The amount of composition which is administered to a patient can vary. Typically, the IV dose may be less than about 10 mL for a 70 Kg patient, with lower doses being preferred.

In addition to the methods disclosed above, another embodiment of preparing a targeted contrast medium comprises combining at least one biocompatible lipid and a gaseous precursor, agitating until gas filled vesicles are formed; adding a targeting ligand to said gas filled vesicles such that the targeting ligand binds to said gas filled vesicle by a covalent bond or non-covalent bond; and agitating until a contrast agent comprising gas filled vesicles and a targeting ligand result. Rather than agitating until gas filled vesicles are formed before adding the targeting ligand, the gaseous precursor may remain a gaseous precursor until the time of use. That is, the gaseous precursor is used to prepare the contrast medium and the precursor is activated in vivo, by temperature for example.

Alternatively, a method of preparing a contrast medium may comprise combining at least one biocompatible lipid and a targeting ligand such that the targeting ligand binds to said lipid by a covalent bond or non-covalent bond, adding a gaseous precursor and agitating until a contrast medium comprising gas filled vesicles and a targeting ligand result. In addition, the gaseous precursor may be added and remain a gaseous precursor until the time of use. That is, the gaseous precursor is used to prepare the contrast medium having gaseous precursor filled vesicles and a targeting ligand which result for use in vivo.

Alternatively, the gaseous precursors may be utilized to create stable gas filled vesicles with targeting ligands which are pre-formed prior to use. In this embodiment, the gaseous precursor and targeting ligand are added to a container housing a suspending and/or stabilizing medium at a temperature below the liquid-gaseous phase transition temperature of the respective gaseous precursor. As the temperature is then exceeded, and an emulsion is formed between the gaseous precursor and liquid solution, the gaseous precursor undergoes transition from the liquid to the gaseous state. As a result of this heating and gas formation, the gas displaces the air in the head space above the liquid suspension so as to form gas filled lipid spheres which entrap the gas of the gaseous precursor, ambient gas for example, air, or coentrap gas state gaseous precursor and ambient air. This phase transition can be used for optimal mixing and stabilization of the contrast medium. For example, the gaseous precursor, perfluorobutane, can be entrapped in the biocompatible lipid or other stabilizing compound, and as the temperature is raised, beyond 4° C. (boiling point of perfluorobutane) stabilizing compound entrapped fluorobutane gas results. As an additional example, the gaseous precursor fluorobutane, can be suspended in an aqueous suspension containing emulsifying and stabilizing agents such as glycerol or propylene glycol and vortexed on a commercial vortexer. Vortexing is commenced at a temperature low enough that the gaseous precursor is liquid and is continued as the temperature of the sample is raised past the phase transition temperature from the liquid to gaseous state. In so doing, the precursor converts to the gaseous state during the microemulsification process. In the presence of the appropriate stabilizing agents, surprisingly stable gas filled vesicles and targeting ligand result.

Accordingly, the gaseous precursors may be selected to form a gas filled vesicle in vivo or may be designed to produce the gas filled vesicle in situ, during the manufacturing process, on storage, or at some time prior to use.

It will be understood by one skilled in the art, once armed with the present disclosure, that the lipids, proteins, polymers and other stabilizing compounds used as starting materials, or the vesicle final products, may be manipulated prior and subsequent to being subjected to the methods contemplated by the present invention. For example, the stabilizing compound such as a biocompatible lipid may be hydrated and then lyophilized, processed through freeze and thaw cycles, or simply hydrated. In preferred embodiments, the lipid is hydrated and then lyophilized, or hydrated, then processed through freeze and thaw cycles and then lyophilized, prior to the formation of gaseous precursor filled vesicles.

According to the methods contemplated by the present invention, the presence of gas, such as and not limited to air, may also be provided by the local ambient atmosphere. The local ambient atmosphere may be the atmosphere within a sealed container, or in an unsealed container, may be the external environment. Alternatively, for example, a gas may be injected into or otherwise added to the container having the aqueous lipid solution or into the aqueous lipid solution itself in order to provide a gas other than air. Gases that are not heavier than air may be added to a sealed container while gases heavier than air may be added to a sealed or an unsealed container. Accordingly, the present invention includes co-entrapment of air and/or other gases along with gaseous precursors.

As already described above in the section dealing with the stabilizing compound, the preferred methods contemplated by the present invention are carried out at a temperature below the gel state to liquid crystalline state phase transition temperature of the lipid employed. By “gel state to liquid crystalline state phase transition temperature”, it is meant the temperature at which a lipid bilayer will convert from a gel state to a liquid crystalline state. See, for example, Chapman et al., J. Biol. Chem. 1974, 249, 2512-2521.

Hence, the stabilized vesicle precursors described above, can be used in the same manner as the other stabilized vesicles used in the present invention, once activated by application to the tissues of a host, where such factors as temperature or pH may be used to cause generation of the gas. It is preferred that this embodiment is one wherein the gaseous precursors undergo phase transitions from liquid to gaseous states at near the normal body temperature of said host, and are thereby activated by the temperature of said host tissues so as to undergo transition to the gaseous phase therein. More preferably still, this method is one wherein the host tissue is human tissue having a normal temperature of about 37° C., and wherein the gaseous precursors undergo phase transitions from liquid to gaseous states near 37° C.

All of the above embodiments involving preparations of the stabilized gas filled vesicles used in the present invention, may be sterilized by autoclave or sterile filtration if these processes are performed before either the gas instillation step or prior to temperature mediated gas conversion of the temperature sensitive gaseous precursors within the suspension. Alternatively, one or more anti-bactericidal agents and/or preservatives may be included in the formulation of the contrast medium, such as sodium benzoate, all quaternary ammonium salts, sodium azide, methyl paraben, propyl paraben, sorbic acid, ascorbylpalmitate, butylated hydroxyanisole, butylated hydroxytoluene, chlorobutanol, dehydroacetic acid, ethylenediamine, monothioglycerol, potassium benzoate, potassium metabisulfite, potassium sorbate, sodium bisulfite, sulfur dioxide, and organic mercurial salts. Such steriliation, which may also be achieved by other conventional means, such as by irradiation, will be necessary where the stabilized microspheres are used for imaging under invasive circumstances, for example, intravascularly or intraperitoneally. The appropriate means of sterilization will be apparent to the artisan instructed by the present description of the stabilized gas filled vesicles and their use. The contrast medium is generally stored as an aqueous suspension but in the case of dried vesicles or dried lipidic spheres the contrast medium may be stored as a dried powder ready to be reconstituted prior to use.

The novel compositions of the present invention, and especially the vesicle compositions, are useful as contrast media in diagnostic imaging, and are also suitable for use in all areas where diagnostic imaging is employed. However, the stabilized vesicles are particularly useful for perfusion imaging.

Diagnostic imaging is a means to visualize internal body regions of a patient. Diagnostic imaging includes, for example, ultrasound (US), magnetic resonance imaging (MRI), nuclear magnetic resonance (NMR), computed tomography (CT), electron spin resonance (ESR); nuclear medicine when the contrast medium includes radioactive material; and optical imaging, particularly with a fluorescent contrast medium. Diagnostic imaging also includes promoting the rupture of the vesicles via the methods of the present invention. For example, ultrasound may be used to visualize the vesicles and verify the localization of the vesicles in certain tissue. In addition, ultrasound may be used to promote rupture of the vesicles once the vesicles reach the intended target, including tissue and/or receptor destination, thus releasing a bioactive agent and/or diagnostic agent.

In accordance with the present invention, there are provided methods of imaging a patient generally, and/or in specifically diagnosing the presence of diseased tissue in a patient. The imaging process of the present invention may be carried out by administering a contrast medium of the invention to a patient, and then scanning the patient using, for example, ultrasound, computed tomography, and/or magnetic resonance imaging, to obtain visible images of an internal region of a patient and/or of any diseased tissue in that region. By region of a patient, it is meant the whole patient or a particular area or portion of the patient. The contrast medium may be particularly useful in providing images of tissue, particularly vascular plaques, but can also be employed more broadly, such as in imaging the vasculature or in other ways as will be readily apparent to those skilled in the art. Cardiovascular region, as that phrase is used herein, denotes the region of the patient defined by the heart and the vasculature leading directly to and from the heart. The phrase vasculature, as used herein, denotes the blood vessels (arteries, veins, etc.) in the body or in an organ or part of the body. The patient can be any type of mammal, but most preferably is a human.

In carrying out the magnetic resonance imaging method of the present invention, the contrast medium can be used alone, or in combination with other diagnostic, therapeutic or other agents. Such other agents include excipients such as flavoring or coloring materials. The magnetic resonance imaging techniques which are employed are conventional and are described, for example, in D. M. Kean and M. A. Smith, Magnetic Resonance Inaging: Principles and Applications, (William and Wilkins, Baltirnore 1986). Contemplated MRI techniques include, but are not limited to, nuclear magnetic resonance (NMR) and electronic spin resonance (ESR). The preferred imaging modality is NMR.

The invention is further demonstrated in the following examples. Examples. 1 to 3 are actual examples and Examples 4 to 6 are prophetic examples. The examples are for purposes of illustration and are not intended to limit the scope of the present invention.

### EXAMPLES

### Example 1

This example is directed to the preparation of targeted gas filled vesicles within the scope of the present invention, as well as a comparison of these targeted vesicles to vesicles of the prior art.

A composition of targeted gas filled lipid vesicles, within the scope of the present invention (referred to herein as Composition 1A), was prepared with 95% dipalmitoylphosphatidylcholine and 5% dipalmitoylphosphatidylserine. This lipid mixture was lyophilized and resuspended in 8:1:1 normal saline:propylene glycol:glycerol at 5 mg/ml. To this suspension was admixed a 10% of dipalmitoylphosphatidylethanolamine labeled with lissamine rhodamine. The resulting mixture was aliquotted into 2 mL serum vials and the headspace was replaced with perfluorobutane. As a control, non-targeted liposomes (referred to herein as Composition 1B) were also prepared from a mixture composed of 82% dipalmitoyl phosphatidylcholine, 10% dipalmitoyl phosphatidic acid and 8% dipalmitoyl phosphatidylethanolamine-PEG5000 with the fluorescent label.

A rabbit model of atherosclerosis was then used in the following experiments. Experimental atherosclerotic lesions were produced in 16 New Zealand White rabbits by balloon de-endothelialization of the infradiaphragmatic aorta and hyperlipidemic diet for 12 weeks. A control section of aorta was used for comparison. The targeted and non-targeted vesicles prepared above were administered by bolus into atherosclerotic rabbits. The animals were imaged using a Phillips SONOS 5500 diagnostic ultrasound machine 15 minutes after the bolus injection.

There was marked enhancement of the atherosclerotic region with Composition 1A, while no improvement was observed with Composition 1B. This is consistent with the targeted vesicles binding to the phosphatidyl serine receptor on entrapped macrophages. Macrophages, however, were shown on histological analysis to contain more rhodamine in samples treated with Composition 1B than with Composition 1A. This discrepancy is accounted for by the greater circulatory lifetime of Composition 1B owing to the PEGylated lipid. The hypothesis was verified when a variant formulation, rhodamine-labeled lipid vesicles, containing 20% DPPS, 10% DPPE-PEG5000 and 70% DPPC, exhibited uptake in macrophages similar to that of Composition 1B.

### Example 2

The procedure of Example 1 was repeated using a vesicle composition composed of 70% DPPC, 20% DPPS and 10% DPPE-PEG5000 doped with DPPE-lissamine rhodamine (hereinafter referred to as Composition 2A). Two rabbits, one with a large artheroma and the other with several smaller artheromas were imaged with Composition 2A and Composition 1B (control). The rabbits both had significantly greater ultrasound contrast enhancement with Composition 2A compared to control, although the difference was less marked in the animal with the smaller plaques. Upon histopathologic analysis, the rhodamine-lissamine labeled vesicles were taken up in macrophages in both animals. Composition 1B was not taken up in either rabbit.

### Example 3

This example is directed to the preparation of a formulation of acoustically active lipospheres and a bioactive agent, within the scope of the present invention.

Paclitaxel (2 g) and soybean oil (3 g) are agitated in a vortex mixer. To this mixture was added a lipid blend of 70 mol percent DPPC, 10 mol percent DPPS and 8 mol percent DPPE-PEG5000 (Avanti Polar Lipids, Alabaster, Ala.) The mixture was stirred for 10 minutes at 50° C., then transferred to a container with normal saline (200 mL) plus 1% Tween-80 and emulsified with a Microfluidizer (10×) at 16,000 psi. The material was subdivided into 1.0 ml aliquots in 1.5 ml vials. The vials were vacuum-evacuated, and the headspace was filled with perfluorobutane. The resulting product was a suspension of drug in oil filled lipospheres containing about 0.9% paclitaxel by weight. The vials were sealed and placed on an Espe Capmix (Hamburg, Germany) and agitated at 2800 rpm for 2 minutes. The final product can be filtered to eliminate a small subgroup of particles over 2 μm.

### Example 4

Example was repeated except that lovastatin (2.5 g) is substituted for paclitaxel.

### Example 5

This example is directed to the preparation of 1,3 di-(methoxy-polyethyleneglycol) n 2-glycerol phosphoserine, which is a PEGylated targeting ligand within the scope of the present invention.

Step A: Preparation of 1,3-di-(methoxy-polyethyleneglycol)_{n 2}-glycerol phosphate.

2-Phosphoglycerol (Aldrich Chemical, Madison, Wis.) is added to two equivalents of tri-butylamine (Aldrich, Madison, Wis.) in dimethylformamide (allinckrodt, St. Louis, Mo.). The dissolved solution is then concentrated in vacuo on a rotoevaporator and vacuum pump to yield an oil. The oil is then redissolved in dimethylformamide and to this solution is added one equivalent methoxy-PEG3400-CONHS ester (Shearwater Polymers, Huntsville, Ala.). The mixture is stirred overnight. The reaction mixture is then concentrated in vacuo followed by dissolution in water. The mixture is then added slowly onto a DEAE-Sephadex G-25 anion exchange column generated in the HCO₃⁻ form. The dilute sample is loaded onto the column followed by rinsing of the column with distilled, deionized water. The column is then developed using a linear gradient comprising 0.01 M triethylammonium bicarbonate (TEA-HCO₃⁻) in the mixing chamber and enriching with 1.0 M TEA-HCO₃⁻ in the reservoir chamber. Samples are collected in appropriate-sized aliquots (tubes) and tested with Na-molybdate in dilute sulfuric acid; a dilute phosphate indicator. The appropriate fractions, appearing blue upon addition of molydate, are then collected and concentrated in vacuo. The product is then washed in methanol and reconcentrated in vacuo to yield a white precipitate. The product in then dissolved in a minimal amount of methanol followed by the dropwise addition of aqueous saturated sodium iodide (Mallinckrodt, St. Louis, Mo.). The precipitate is then collected by centrifugation and decanting of the methanol. Washing with methanol followed by repeat centrifugation affords the sodium salt of 1,3-di-(methoxy-polyethyleneglycol)n 2-glycerol phosphate.

Step B: Preparation of 1,3 di-(methoxy-polyethyleneglycol) n 2-glycerol phosphoserine.

To the product of Step A is added 2 equivalents of tributylamine in DMF followed by concentration in vacuo. The oil (the tributylammonium salt of 1,3 di-(methoxy-polyethyleneglycol) n 2-glycerol phosphate is then dissolved in dry DMF and chilled to 0° C. followed by addition of one equivalent of carbonyldiimidazole (CDI) (Aldrich, Milwaukee, Wis.). The solution is allowed to stir for one hour followed by the dropwise addition of two equivalents Fmoc-serine dissolved in DMF. After addition, the solution is allowed to equilibrate to room temperature and stirring continued for four hours. After stirring, 20 volume % (1 ml for every 5 mL of reaction mixture) of piperidine is added and the solution is allowed to stir an additional 20 minutes. The mixture is then concentrated in vacuo followed by dissolution in water and purification once again by anion exchange chromatography. Purification of the phosphoserine analog is obtained by reverse-phase chromatography.

### Example 6

This example is directed to the preparation of vesicles including a targeted bioconjugate, which is within the scope of the present invention.

Vesicles targeted for atherosclerotic plaque will be prepared from a lipid blend composed of the lipids employed in Composition 2A from Example 2, except that the DPPS will be replaced with the final bioconjugate product of Example 5.

### Example 7

This example is directed to the preparation of acoustically active lipospheres containing the phosphoserine bioconjugates, within the scope of the present invention.

Vesicles targeted for atherosclerotic plaque will be prepared from a lipid blend composed of the lipids employed in Example 3, except that the DPPS will be replaced with the final bioconjugate product of Example 5.

The disclosures of each patent, patent application and publication cited or described in this document are hereby incorporated herein by reference, in their entirety.

Various modifications of the invention, in addition to those described herein, will be apparent to those skilled in the art from the foregoing description. Such modifications are also intended to fall within the scope of the appended claims.

1. A method for providing an image of an internal region of a patient having a vascular plaque, wherein the method comprises (i) administering to the patient a contrast agent comprising, in an aqueous carrier, targeted vesicles formulated from a lipid or protein, a gas or gaseous precursor, a targeting ligand, and optionally, an oil, wherein said targeting ligand targets cells or receptors associated with vascular plaque and comprises an acid moiety; and (ii) scanning the patient using ultrasound to obtain a visible image of the region.

2. A method according to claim 1 wherein said contrast agent comprises lipid vesicles.

3. A method according to claim 2 wherein said lipid comprises a phospholipid.

4. A method according to claim 3 wherein said phospholipid is selected from the group consisting of a phosphatidylcholine and a phosphatidylethanolamine.

5. A method according to claim 4 wherein said phosphatidylcholine is selected from the group consisting of dioleoylphosphatidylcholine, dimyristoyl-phosphatidylcholine, dipalmitoylphosphatidylcholine and distearoylphosphatidylcholine.

6. A method according to claim 5 wherein said phosphatidylcholine comprises dipalmitoylphosphatidylcholine.

7. A method according to claim 4 wherein said phosphatidylethanol-amine is selected from the group consisting of dipalmitoylphosphatidylethanolamine, dioleoylphosphatidylethanolamine, N-succinyldioleoylphosphatidylethanolamine and 1-hexadecyl-2-palmitoylglycerophosphoethanolamine.

8. A method according to claim 7 wherein said phosphatidylethanol-amine comprises dipalmitoylphosphatidylethanolamine.

9. A method according to claim 2 wherein said lipid further comprises a polymer.

10. A method according to claim 9 wherein said polymer comprises a hydrophilic polymer.

11. A method according to claim 10 wherein said polymer comprises polyethylene glycol.

12. A method according to claim 1 wherein said vesicles comprise protein vesicles.

13. A method according to claim 12 wherein said protein comprises albumin.

14. A method according to claim 1 wherein said gas or gaseous precursor comprises a fluorinated compound.

15. A method according to claim 14 wherein said fluorinated compound is a perfluorocarbon compound.

16. A method according to claim 15 wherein said perfluorocarbon is selected from the group consisting of perfluoromethane, perfluoroethane, perfluoropropane, perfluorobutane, perfluorocyclobutane, perfluoropentane, perfluorohexane, perfluoroheptane, perfluorooctane, perfluorononane, perfluorodecane, perfluorodecalin, perfluoroundecane, perfluorododecane, and mixtures thereof.

17. A method according to claim 16 wherein said perfluorocarbon is selected from the group consisting of perfluoropropane, perfluorobutane, perfluorocyclobutane, perfluoropentane, perfluorohexane, and mixtures thereof.

18. A method according to claim 17 wherein said perfluorocarbon is perfluorobutane.

19. A method according to claim 1 wherein said targeting ligand comprises a lipid containing said acid moiety.

20. A method according to claim 19 wherein said targeting ligand comprises an acidic phospholipid.

21. A method according to claim 20 wherein said targeting ligand is a diacyl phospholipid.

22. A method according to claim 20 wherein said targeting ligand is selected from the group consisting of phosphatidic acids, phosphatidyl serines and phosphatidylinositols.

23. A method according to claim 22 wherein said targeting ligand is a phosphatidic acid which is dipalmitoylphosphatidic acid.

24. A method according to claim 22 wherein said targeting ligand is a phosphatidyl serine which is dipalmitoylphosphatidylserine.

25. A method according to claim 1 wherein said contrast agent further comprises a low viscosity oil.

26. A method according to claim 25 wherein said oil is selected from the group consisting of silicone oiL cod liver oil, triacetin, mineral oil, plant oil, oil comprising fluorinated triglycerides, biocompatible saturated fatty acids, biocompatible unsaturated fatty acids, biocompatible partially hydrogenated fatty acids, silicon-based oils, and synthetic oil.

27. A method according to claim 2 wherein said vesicles are selected from the group consisting of micelles and liposomes.

28. A method according to claim 27 wherein said lipid vesicles are selected from the group consisting of unilamellar lipid vesicles, oligolamellar lipid vesicles and multilamellar lipid vesicles.

29. A method according to claim 28 wherein said lipids are in the form of monolayers or bilayers.

30. A method for diagnosing the presence of a vascular plaque in a patient, wherein the method comprises (i) administering to the patient a contrast agent comprising, in an aqueous carrier, targeted vesicles formulated from a lipid or protein, a gas or gaseous precursor, a targeting ligand, and optionally, an oil, wherein said targeting ligand targets cells or receptors associated with vascular plaque and comprises an acid moiety; and (ii) scanning the patient using ultrasound to obtain a visible image of any plaque in the patient.

31. A method according to claim 30 wherein said contrast agent comprises lipid vesicles.

32. A method according to claim 31 wherein said lipid comprises a phospholipid.

33. A method according to claim 32 wherein said phospholipid is selected from the group consisting of a phosphatidylcholine and a phosphatidylethanolamine.

34. A method according to claim 33 wherein said phosphatidylcholine is selected from the group consisting of dioleoylphosphatidylcholine, dimyristoyl-phosphatidylcholine, dipalmitoylphosphatidylcholine and distearoylphosphatidylcholine.

35. A method according to claim 34 wherein said phosphatidylcholine comprises dipalmitoylphosphatidylcholine.

36. A method according to claim 33 wherein said phosphatidylethanol-amine is selected from the group consisting of dipalmitoylphosphatidylethanolamine, dioleoylphosphatidylethanolamine, N-succinyldioleoylphosphatidylethanolamine and 1-hexadecyl-2-palmitoylglycerophosphoethanolamine.

37. A method according to claim 36 wherein said phosphatidylethanol-amine comprises dipalmitoylphosphatidylethanolamine.

38. A method according to claim 31 wherein said lipid further comprises a polymer.

39. A method according to claim 38 wherein said polymer comprises a hydrophilic polymer.

40. A method according to claim 39 wherein said polymer comprises polyethylene glycol.

41. A method according to claim 30 wherein said vesicles comprise protein vesicles.

42. A method according to claim 41 wherein said protein comprises albumin.

43. A method according to claim 30 wherein said gas or gaseous precursor is a fluorinated compound.

44. A method according to claim 43 wherein said fluorinated compound is a perfluorocarbon compound.

45. A method according to claim 44 wherein said perfluorocarbon compound contains from 1 to about 12 carbons.

46. A method according to claim 45 wherein said perfluorocarbon compound contains from about 3 to about 6 carbons.

47. A method according to claim 46 wherein said perfluorocarbon compound contains about 4 carbons.

48. A method according to claim 30 wherein said targeting ligand comprises a lipid containing said acid moiety.

49. A method according to claim 48 wherein said targeting ligand comprises an acidic phospholipid.

50. A method according to claim 49 wherein said targeting ligand is a diacyl phospholipid.

51. A method according to claim 49 wherein said targeting ligand is selected from the group consisting of phosphatidic acids, phosphatidyl serines and phosphatidylinositols.

52. A method according to claim 51 wherein said targeting ligand is a phosphatidic acid which is dipalmitoylphosphatidic acid.

53. A method according to claim 51 wherein said targeting ligand is a phosphatidyl serine which is dipalmitoylphosphatidylserine.

54. A method according to claim 30 wherein said contrast agent further comprises a low viscosity oil.

55. A method according to claim 54 wherein said oil is selected from the group consisting of silicone oil, cod liver oil, mineral oil, triacetin, plant oil, oil comprising fluorinated triglycerides, biocompatible saturated fatty acids, biocompatible unsaturated fatty acids, biocompatible partially hydrogenated fatty acids, silicon-based oils, and synthetic oil.

56. A method according to claim 31 wherein said vesicles are selected from the group consisting of micelles and liposomes.

57. A method according to claim 56 wherein said lipid vesicles are selected from the group consisting of unilamellar lipid vesicles, oligolamellar lipid vesicles and multilamellar lipid vesicles.

58. A method according to claim 57 wherein said lipids are in the form of monolayers or bilayers.

59. A method for the therapeutic delivery in vivo of a bioactive agent to a region in a patient having a vascular plaque, wherein the method comprises administering to a patient a therapeutically effective amount of a formulation comprising, in combination with a bioactive agent, a composition which comprises vesicles formulated from a lipid or protein, a gas or gaseous precursor, a targeting ligand, and optionally, an oil, wherein said targeting ligand targets cells or receptors associated with vascular plaque and comprises an acid moiety.

60. A method according to claim 59 wherein said bioactive agent is selected from the group consisting of anti-thrombolytic agents, statins, anti-cancer agents, and radioactive materials.

61. A method according to claim 60 further comprising applying ultrasonic energy to the patient to release said bioactive agent from said targeted vesicles.

62. A method according to claim 61, wherein said ultrasonic energy causes said vesicles to rupture.

63. A method according to claim 59, further comprising the step of scanning the patient with diagnostic imaging to visualize said vesicles at the target site.

64. A method of dissolving plaque in a blood vessel comprising (i) administering to a patient, by intravenous injection, a targeted vesicle composition comprising vesicles formulated from a lipid or protein, a gas or gaseous precursor, a targeting ligand, and optionally, an oil, wherein said targeting ligand targets cells or receptors associated with vascular plaque and comprises an acid moiety; (ii) scanning said patient with diagnostic imaging to visualize said plaque; and (iii) applying ultrasonic energy to said plaque.

65. A method according to claim 64 wherein said ultrasonic energy in step (iii) is pulsed ultrasound.

66. A method according to claim 64 wherein said targeting ligand comprises a lipid containing said acid moiety.

67. A method according to claim 66 wherein said targeting ligand comprises an acidic phospholipid.

68. A method according to claim 67 wherein said targeting ligand is a diacyl phospholipid.

69. A method according to claim 67 wherein said targeting ligand is selected from the group consisting of phosphatidic acids, phosphatidyl serines and phosphatidylinositols.

70. A method according to claim 69 wherein said targeting ligand is a phosphatidic acid which is dipalmitoylphosphatidic acid.

71. A method according to claim 69 wherein said targeting ligand is a phosphatidyl serine which is dipalmitoylphosphatidylserine.

72. A method for providing an image of an internal region of a patient having a vascular plaque, wherein the method comprises (i) administering to the patient a contrast agent comprising, in an aqueous carrier, targeted vesicles formulated from a lipid or polymer, a gas or gaseous precursor, a targeting ligand, and optionally, an oil, wherein said targeting ligand targets cells or receptors associated with vascular plaque and comprises a phosphorylated serine moiety; and (ii) scanning the patient using ultrasound to obtain a visible image of the region.

73. A method for diagnosing the presence of a vascular plaque in a patient, wherein the method comprises (i) administering to the patient a contrast agent comprising, in an aqueous carrier, targeted vesicles formulated from a lipid or polymer, a gas or gaseous precursor, a targeting ligand, and optionally, an oil, wherein said targeting ligand targets cells or receptors associated with vascular plaque and comprises a phosphorylated serine moiety; and (ii) scanning the patient using ultrasound to obtain a visible image of any plaque in the patient.

74. A method for the therapeutic delivery in vivo of a bioactive agent to a region in a patient having a vascular plaque, wherein the method comprises administering to a patient a therapeutically effective amount of a formulation comprising, in combination with a bioactive agent, a composition which comprises vesicles formulated from a lipid or polymer, a gas or gaseous precursor, a targeting ligand, and optionally, an oil, wherein said targeting ligand targets cells or receptors associated with vascular plaque and comprises a phosphorylated serine moiety.

75. A composition for use in targeting an internal region of a patient having vascular plaque, wherein the composition comprises vesicles formulated from a lipid or protein, a gas or gaseous precursor, a targeting ligand, and optionally, an oil, wherein said targeting ligand targets cells or receptors associated with vascular plaque and comprises an acid moiety.

76. A composition according to claim 75 which comprises lipid vesicles.

77. A composition according to claim 76 wherein said lipid comprises a phospholipid.

78. A composition according to claim 77 wherein said phospholipid is selected from the group consisting of a phosphatidylcholine and a phosphatidylethanolamine.

79. A composition according to claim 78 wherein said phosphatidylcholine is selected from the group consisting of dioleoylphosphatidylcholine, dimryristoylphosphatidylcholine, dipalmitoylphosphatidylcholine and distearoylphosphatidylcholine.

80. A composition according to claim 79 wherein said phosphatidylcholine comprises dipalmitoylphosphatidylcholine.

81. A composition according to claim 78 wherein said phosphatidylethanolamine is selected from the group consisting of dipalmitoyl-phosphatidylethanolamine, dioleoylphosphatidylethanolamine, N-succinyldioleoyl-phosphatidylethanolamine and 1-hexadecyl-2-palmitoylglycerophosphoethanolamine.

82. A composition according to claim 81 wherein said phosphatidylethanolamine comprises dipalmitoylphosphatidylethanolamine.

83. A composition according to claim 76 wherein said lipid further comprises a polymer.

84. A composition according to claim 83 wherein said polymer comprises a hydrophilic polymer.

85. A composition according to claim 84 wherein said polymer comprises polyethylene glycol.

86. A composition according to claim 75 which comprises protein vesicles.

87. A composition according to claim 86 wherein said protein comprises albumin.

88. A composition according to claim 75 wherein said gas or gaseous precursor comprises a fluorinated compound.

89. A composition according to claim 88 wherein said fluorinated compound is a perfluorocarbon compound.

90. A composition according to claim 89 wherein said perfluorocarbon is selected from the group consisting of perfluoromethane, perfluoroethane, perfluoropropane, perfluorobutane, perfluorocyclobutane, perfluoropentane, perfluorohexane, perfluoroheptane, perfluorooctane, perfluorononane, perfluorodecane, perfluorodecalin, perfluoroundecane, perfluorododecane, and mixtures thereof.

91. A composition according to claim 90 wherein said perfluorocarbon is selected from the group consisting of perfluoropropane, perfluorobutane, perfluorocyclobutane, perfluoropentane, perfluorohexane, and mixtures thereof.

92. A composition according to claim 91 wherein said perfluorocarbon is perfluorobutane.

93. A composition according to claim 75 wherein said targeting ligand comprises a lipid containing said acid moiety.

94. A composition according to claim 93 wherein said targeting ligand comprises an acidic phospholipid.

95. A composition according to claim 94 wherein said targeting ligand is a diacyl phospholipid.

96. A composition according to claim 94 wherein said targeting ligand is selected from the group consisting of phosphatidic acids, phosphatidyl serines and phosphatidylinositols.

97. A composition according to claim 96 wherein said targeting ligand is a phosphatidic acid which is dipalmitoylphosphatidic acid.

98. A composition according to claim 96 wherein said targeting ligand is a phosphatidyl serine which is dipalmitoylphosphatidylserine.

99. A composition according to claim 75 which further comprises a low viscosity oil.

100. A composition according to claim 99 wherein said oil is selected from the group consisting of silicone oiL cod liver oil, mineral oil, triacetin, plant oil, oil comprising fluorinated triglycerides, biocompatible saturated fatty acids, biocompatible unsaturated fatty acids, biocompatible partially hydrogenated fatty acids, silicon-based oils, and synthetic oil.

101. A composition according to claim 76 wherein said vesicles are selected from the group consisting of micelles and liposomes.

102. A composition according to claim 101 wherein said lipid vesicles are selected from the group consisting of unilamellar lipid vesicles, oligolamellar lipid vesicles and multilamellar lipid vesicles.

103. A composition according to claim 102 wherein said lipids are in the form of monolayers or bilayers.

104. A composition according to claim 75 which further comprises a bioactive agent.

105. A composition according to claim 104 wherein said bioactive agent is selected from the group consisting of anti-thrombolytic drugs, statins, anti-cancer agents, and radioactive materials.

106. A composition for use in targeting an internal region of a patient having vascular plaque, wherein the composition comprises vesicles formulated from a lipid or polymer, a gas or gaseous precursor, a targeting ligand, and optionally, an oil, wherein said targeting ligand targets cells or receptors associated with vascular plaque and comprises a phosphorylated serine moiety.

107. A formulation for therapeutic or diagnostic use in a patient having a vascular plaque, wherein the formulation comprises, in combination with a bioactive agent, a composition comprising vesicles formulated from a lipid or protein, a gas or gaseous precursor, a targeting ligand, and optionally, an oil, wherein said targeting ligand targets cells or receptors associated with vascular plaque and comprises an acid moiety.

108. A formulation according to claim 107 wherein said vesicles comprise lipid vesicles.

109. A formulation according to claim 108 wherein said vesicles are selected from the group consisting of micelles and liposomes.

110. A formulation for therapeutic or diagnostic use in a patient having a vascular plaque, wherein the formulation comprises, in combination with a bioactive agent, a composition comprising vesicles formulated from a lipid or polymer, a gas or gaseous precursor, a targeting ligand, and optionally, an oil, wherein said targeting ligand targets cells or receptors associated with vascular plaque and comprises a phosphorylated serine moiety.

111. A process for the preparation of a composition for use in targeting a region in a patient having a vascular plaque, wherein the process comprises combining together a lipid or protein, a gas or gaseous precursor, a targeting ligand, and optionally, an oil, wherein said targeting ligand targets cells or receptors associated with vascular plaque and comprises an acid moiety.

112. A process according to claim 111 wherein said composition comprises vesicles.

113. A process for the preparation of a composition for use in targeting a region in a patient having a vascular plaque, wherein the process comprises combining together a lipid or polymer, a gas or gaseous precursor, a targeting ligand, and optionally, an oil, wherein said targeting ligand targets cells or receptors associated with vascular plaque and comprises a phosphorylated serine moiety.

114. A process for the preparation of a formulation for therapeutic or diagnostic use in a patient having a vascular plaque, wherein the process comprises combining together a bioactive agent, a lipid or protein, a gas or gaseous precursor, a targeting ligand, and optionally, an oil, wherein said targeting ligand targets cells or receptors associated with vascular plaque and comprises an acid moiety.

115. A process according to claim 114 wherein said formulation comprises lipid vesicles.

116. A process for the preparation of a formulation for therapeutic or diagnostic use in a patient having a vascular plaque, wherein the process comprises combining together a bioactive agent, a lipid or polymer, a gas or gaseous precursor, a targeting ligand, and optionally, an oil, wherein said targeting ligand targets cells or receptors associated with vascular plaque and comprises a phosphorylated serine moiety.
